(19) United States
(12) Patent Application Publication
(10) Pub. No.: US 2025/0197918 A1
(43) Pub. Date: Jun. 19, 2025

(54) PREPARATION AND USE OF BLOCKED SUBSTRATES

(71) Applicant: Illumina, Inc., San Diego, CA (US)

(72) Inventors: Xin Sheng, San Diego, CA (US); Jian-Sen Li, San Diego, CA (US); Aaron Aslanian, San Diego, CA (US); Melissa Carpenter, San Diego, CA (US); Thomas Richard Gros, Encinitas, CA (US); Nassim Ataii, Foster City, CA (US); Thais Takei, San Diego, CA (US); Maria Annabelle Nulud Mesina-Gross, San Diego, CA (US)

(21) Appl. No.: 18/846,089

(22) PCT Filed: Aug. 28, 2023
(86) PCT No.: PCT/US2023/072992

§ 371 Date: Sep. 11, 2024

Related U.S. Application Data
(60) Provisional application No. 63/387,152, filed on Dec. 13, 2022.
(60) Provisional application No. 63/373,832, filed on Aug. 29, 2022.

(51) Int. Cl. C12Q 1/6806

(52) U.S. Cl. CPC C12Q 1/6806

(57) ABSTRACT

Some embodiments of the methods and compositions provided herein relate to blocked substrates in which non-specific binding of nucleic acids to the substrate is reduced. Some embodiments include use of carrier nucleic acids. More embodiments include the use of beads contacted with an oligonucleotide, such as an oligonucleotide containing one or more phosphorothioate bonds. Such substrates are useful in methods for obtaining long-read information from short reads of a target nucleic acid.

### RELATED APPLICATIONS

This application claims priority to U.S. Prov. App. No. 63/387,152 filed Dec. 13, 2022, and to U.S. Prov. App. No. 63/373,832 filed Aug. 29, 2022 which are each incorporated by reference herein in its entirety.

### REFERENCE TO SEQUENCE LISTING

The present application is being filed along with a Sequence Listing in electronic format. The Sequence Listing is provided as a file entitled SEQLIST_ILLINC733WO, created Aug. 21, 2023, which is approximately 6387 bytes in size. The information in the electronic format of the Sequence Listing is incorporated herein by reference in its entirety.

### FIELD OF THE INVENTION

Some embodiments of the methods and compositions provided herein relate to blocked substrates in which non-specific binding of nucleic acids to the substrate is reduced. Some embodiments include use of carrier nucleic acids. More embodiments include the use of beads contacted with an oligonucleotide, such as an oligonucleotide containing one or more phosphorothioate bonds. Such substrates are useful in methods for obtaining long-read information from short reads of a target nucleic acid.

### BACKGROUND OF THE INVENTION

Current protocols for next-generation sequencing (NGS) of nucleic acid samples routinely employ a sample preparation process that converts DNA or RNA into a library of fragmented, sequenceable templates. Sample preparation methods often require multiple steps, material transfers, and expensive instruments to effect fragmentation, and therefore are often difficult, tedious, expensive, and inefficient.

In one approach, nucleic acid fragment libraries may be prepared using a transposome-based method where two transposon end sequences, one linked to a tag sequence, and a transposase form a transposome complex. The transposome complexes are used to fragment and tag target nucleic acids in solution to generate a sequencer-ready tagmented library. The transposome complexes may be immobilized on a solid surface, such as through a biotin appended at the 5′ end of one of the two end sequences. Use of immobilized transposomes provides significant advantages over solution-phase approaches by reducing hands-on and overall library preparation time, cost, and reagent requirements, lowering sample input requirements, and enabling the use of unpurified or degraded samples as a starting point for library preparation. However, certain portions of a genome may be underrepresented in libraries prepared using transposomes.

### SUMMARY OF THE INVENTION

Some embodiments of the methods and compositions provided herein include a method for stabilizing a nucleic acid sample, comprising contacting the nucleic acid sample with (i) an oligonucleotide, wherein the oligonucleotide comprises a backbone comprising a phosphorothioate bond; or (ii) carrier nucleic acids.

In some embodiments, the nucleic acid sample is contacted with the oligonucleotide. In some embodiments, the oligonucleotide comprises 20, 40, 60 or more consecutive nucleotides. In some embodiments, the oligonucleotide comprises or consists of 60 consecutive nucleotides.

In some embodiments, at least 50%, 70%, 90%, or 95% of the backbone comprises phosphorothioate bonds. In some embodiments, 100% of the backbone comprises phosphorothioate bonds.

In some embodiments, the oligonucleotide comprises a sequence lacking the capability of forming a hairpin structure at a temperature less than 25° C., 0° C., or less than −40° C.

In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO:02.

In some embodiments, the oligonucleotide comprises DNA. In some embodiments, the oligonucleotide comprises RNA. In some embodiments, the oligonucleotide is single-stranded.

In some embodiments, the nucleic acid sample is contacted with the carrier nucleic acids; wherein the nucleic acid sample comprises a target nucleic acid. In some embodiments, the carrier nucleic acids and the target nucleic acid are each derived from a genome of an organism of a kingdom, phylum, class, order, family, genus or species different from each other. In some embodiments, the carrier nucleic acids are derived from a genome of a fish. In some embodiments, the carrier nucleic acids comprise salmon sperm DNA, RNA, or siRNA.

In some embodiments, the carrier nucleic acids have an average length less than 5000 consecutive nucleotides. In some embodiments, the carrier nucleic acids have an average length in a range from 100 to 5000 consecutive nucleotides. In some embodiments, the carrier nucleic acids have an average length in a range from 100 to 1000 consecutive nucleotides. In some embodiments, the carrier nucleic acids have an average length greater than 5000 consecutive nucleotides. In some embodiments, the carrier nucleic acids have an average length in a range from 5000 to 10,000 consecutive nucleotides.

In some embodiments, the target nucleic acid comprises an adaptor. In some embodiments, the adaptor comprises a nucleotide sequence selected from a P5 sequence (SEQ ID NO: 05), a P7 sequence (SEQ ID NO:06), or a complement thereof. In some embodiments, the target nucleic acid has a concentration less than 10 nM, 100 pM, 20 pM, or 5 pM. In some embodiments, the target nucleic acid comprises (i) a bacteriophage nucleic acid; optionally, wherein the bacteriophage is a PhiX; or (ii) a mammalian nucleic acid, such as human. In some embodiments, the target nucleic acid comprises DNA. In some embodiments, the target nucleic acid is single-stranded.

In some embodiments, the nucleic acid has a concentration less than 500 nM, 100 nM, 10 nM, 100 pM, 20 pM, or 5 pM.

Some embodiments also include sequencing the nucleic acid sample, wherein sequence data obtained from the nucleic acid sample is improved compared to a nucleic acid same lacking the oligonucleotide or carrier nucleic acids; optionally, wherein the improvement comprises an improved sequencing metric selected from N50, GC bias, percentage duplicated reads, redundancy of reads, error rate, CFR intensity, percentage alignment, percentage pass filter, cluster pass filter, and average cluster density.

Some embodiments of the methods and compositions provided herein include a method for reducing non-specific nucleic acid binding to a substrate, comprising: contacting the substrate with an oligonucleotide, wherein the oligonucleotide comprises a backbone comprising a phosphorothioate bond, and wherein non-specific nucleic acid binding to the substrate is reduced compared to a substrate not contacted with the oligonucleotide.

In some embodiments, the substrate comprises a bead. In some embodiments, the substrate comprises a magnetic bead. In some embodiments, an agent is bound to a surface of the substrate, wherein the agent is selected from streptavidin, biotin, or a derivative thereof.

In some embodiments, the contacting is for a period greater than 30 minutes. In some embodiments, the contacting is for a period greater than 1 hour, 6 hours, or 12 hours. In some embodiments, the contacting is performed at room temperature. In some embodiments, the contacting is performed at about 4° C.

Some embodiments also include contacting the substrate with a plurality of transposomes. Some embodiments also include contacting the substrate with genomic DNA. In some embodiments, the nucleic acid comprises DNA. In some embodiments, the nucleic acid comprises genomic DNA.

In some embodiments, the oligonucleotide comprises 20, 40, 60 or more consecutive nucleotides. In some embodiments, the oligonucleotide comprises or consists of 60 consecutive nucleotides.

In some embodiments, at least 50%, 70%, 90%, or 95% of the backbone comprises phosphorothioate bonds. In some embodiments, 100% of the backbone comprises phosphorothioate bonds.

In some embodiments, the oligonucleotide comprises a sequence lacking the capability of forming a hairpin structure at a temperature less than 25° C., 0° C., or less than −40° C.

In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO:02.

In some embodiments, the oligonucleotide comprises DNA. In some embodiments, the oligonucleotide comprises RNA. In some embodiments, the oligonucleotide is single-stranded.

Some embodiments of the methods and compositions provided herein include a method of normalizing a level of non-specific nucleic acid binding to a plurality of substrates, comprising performing any one of the foregoing methods for reducing non-specific nucleic acid binding to a substrate. In some embodiments, the plurality of substrates comprises beads from different lots.

Some embodiments of the methods and compositions provided herein include a composition prepared by any one of the foregoing methods for reducing non-specific nucleic acid binding to a substrate. In some embodiments, the substrate comprises a plurality of beads.

Some embodiments of the methods and compositions provided herein include a blocked bead composition comprising a magnetic bead in contact with an oligonucleotide, wherein the oligonucleotide comprises a backbone comprising a phosphorothioate bond, and wherein non-specific nucleic acid binding to the blocked bead is reduced compared to non-specific nucleic acid binding to a bead not in contact with the oligonucleotide.

In some embodiments, an agent is bound to a surface of the bead, wherein the agent is selected from streptavidin, biotin, or a derivative thereof.

In some embodiments, the nucleic acid comprises DNA. In some embodiments, the nucleic acid comprises genomic DNA.

In some embodiments, the oligonucleotide comprises 20, 40, 60 or more consecutive nucleotides.

In some embodiments, at least 50%, 70%, 90%, or 95% of the backbone comprises phosphorothioate bonds. In some embodiments, 100% of the backbone comprises phosphorothioate bonds.

In some embodiments, the oligonucleotide comprises a sequence lacking the capability of forming a hairpin structure at a temperature less than 25° C., 0° C. or −40° C.

In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO:02.

In some embodiments, the oligonucleotide comprises DNA. In some embodiments, the oligonucleotide comprises RNA. In some embodiments, the oligonucleotide is single-stranded.

Some embodiments also include a transposome bound to the bead.

Some embodiments of the methods and compositions provided herein include a method for preparing a nucleic acid library, comprising:
(a) obtaining a plurality of transposomes comprising transposon adaptors, wherein the plurality of transposomes are immobilized on a solid support, in some embodiments, the solid support comprises a bead, in some embodiments, the bead comprises any one of the foregoing blocked bead compositions; (b) contacting a plurality of nucleic acid fragments with the plurality of transposomes to obtain a plurality of polynucleotides; (c) amplifying the plurality of polynucleotides to obtain amplified polynucleotides; and (d) adding library adapters to each end of the amplified polynucleotides, thereby obtaining the nucleic acid library. In some embodiments, step (c) and/or (d) comprises use of any one of the foregoing blocked bead compositions.

In some embodiments, the plurality of the transposomes is immobilized on the bead at a density such that an average length of the plurality of polynucleotides greater than about 1 kbp, 2 kbp, 5 kbp, 10 kbp, 15 kbp, 20 kbp, or 40 kbp.

In some embodiments, the number of transposomes immobilized on the bead is no more than about 100 transposomes, 50 transposomes, 40 transposomes, 30 transposomes, 20 transposomes, or 10 transposomes. In some embodiments, the number of transposomes immobilized on the bead is no more than about 30 transposomes.

In some embodiments, the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp.

In some embodiments, the plurality of the transposomes immobilized on the bead comprise an activity in a range from about 0.05 AU/μl to about 0.25 AU/μl. In some embodiments, the plurality of the transposomes immobilized on the bead comprise an activity of about 0.075 AU/μl.

In some embodiments, the transposon adapters comprise the same sequence. In some embodiments, the transposon adapters comprise the nucleotide sequence: (SEQ ID NO:01).

In some embodiments, the transposomes of the plurality of transposomes are the same In some embodiments, the transposomes of the plurality of transposomes are B15 transposomes.

In some embodiments, step (c) comprises a mutagenesis PCR, such that mutations are introduced into amplified polynucleotides. In some embodiments, the mutagenesis PCR comprises amplifying the plurality of polynucleotides with a low bias DNA polymerase, and/or with a nucleotide analogue. In some embodiments, the nucleotide analogue comprises dPTP, and/or 8-oxo-dGTP. In some embodiments, the low bias DNA polymerase is a Thermococcal polymerase, or a functional derivative thereof. In some embodiments, the Thermococcal polymerase is derived from a Thermococcal strain selected from the group consisting of T. kodakarensis, T. siculi, T. celer and T. sp KS-1. In some embodiments, the mutagenesis PCR comprises no more than 12 cycles, 10 cycles, 9 cycles, 8 cycles, 7 cycles, 6 cycles, 5 cycles, 4 cycles, 3 cycles, or 2 cycles.

In some embodiments, a first end of a polynucleotide of the plurality of polynucleotides is capable of annealing to a second end of the polynucleotide of the plurality of polynucleotides; and/or, wherein a first end of an amplified polynucleotide is capable of annealing to a second end of the amplified polynucleotide.

In some embodiments, step (c) further comprises a suppression PCR. In some embodiments, the suppression PCR comprises use of a single amplification primer, and/or the suppression PCR comprises no more than 16 cycles, 14 cycles, 10 cycles, 9 cycles, 8 cycles, 7 cycles, 6 cycles, 5 cycles, 4 cycles, 3 cycles, or 2 cycles. In some embodiments, the amplified polynucleotides have an average length greater than about 1 kbp, 2 kbp, 3 kbp, 4 kbp, 5 kbp, 10 kbp, 15 kbp, or 20 kbp.

Some embodiments also include enriching for target nucleic acids in the amplified polynucleotides. In some embodiments, the enriching comprises hybridizing a plurality of selection probes with the amplified polynucleotides, wherein the plurality of selection probes is capable of specifically hybridizing with the target nucleic acids. In some embodiments, the plurality of selection probes lacks sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon.

Some embodiments also include amplifying the target nucleic acids.

In some embodiments, step (d) comprises contacting the amplified polynucleotides with an additional plurality of transposomes. In some embodiments, the additional plurality of transposomes comprise transposon adapters comprising (i) indexes, and/or (iii) sequencing primer binding sites.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Some embodiments also include amplifying the target polynucleotides.

In some embodiments, an amount of the plurality of nucleic acid fragments is less than about 100 ng, 50 ng, 30 ng, 20 ng, 10 ng, 5 ng, or 1 ng.

In some embodiments, the plurality of nucleic acid fragments is mammalian. In some embodiments, the plurality of nucleic acid fragments is human. In some embodiments, the plurality of nucleic acid fragments comprises genomic DNA.

Some embodiments of the methods and compositions provided herein include a method for determining a sequence of a target nucleic acid, comprising: performing any one of the foregoing methods for preparing a nucleic acid library; sequencing the library of nucleic acids to obtain sequence reads; and assembling sequence reads to obtain the sequence of a target nucleic acid.

In some embodiments, the assembling comprises comparing the sequence reads to a reference sequence. In some embodiments, the reference sequence is obtained from the same nucleic acid sample as the plurality of nucleic acid fragments.

In some embodiments, one or more of steps (a)-(d) is performed in a reaction vessel, and the method further comprises adding carrier nucleic acids to the reaction vessel.

Some embodiments of the methods and compositions provided herein include a method for preparing a nucleic acid library, comprising: (a) obtaining a plurality of transposomes comprising transposon adaptors, wherein the plurality of transposomes is immobilized on a bead, wherein the transposomes of the plurality of transposomes are the same, in some embodiments, the bead comprises any one of the foregoing blocked bead compositions; (b) contacting a plurality of nucleic acid fragments with the plurality of transposomes to obtain a plurality of polynucleotides, wherein the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; (c) amplifying the plurality of polynucleotides to obtain amplified polynucleotides by: (i) performing a mutagenesis PCR, such that mutations are introduced into amplified polynucleotides, and (ii) performing a suppression PCR; and (d) adding library adapters to each end of the amplified polynucleotides by contacting the amplified polynucleotides with an additional plurality of transposomes, thereby obtaining the nucleic acid library.

Some embodiments also include enriching for target nucleic acids in the amplified polynucleotides. In some embodiments, the enriching comprises hybridizing a plurality of selection probes with the amplified polynucleotides, wherein the plurality of selection probes is capable of specifically hybridizing with the target nucleic acids; optionally, wherein the plurality of selection probes lacks sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon.

Some embodiments also include amplifying the target nucleic acids.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Some embodiments also include amplifying the target polynucleotides.

In some embodiments, one or more of steps (a)-(d) is performed in a reaction vessel, and the method further comprises adding carrier nucleic acids to the reaction vessel.

Some embodiments of the methods and compositions provided herein include a method of sequencing a target nucleic acid, comprising: (a) obtaining a sample comprising the target nucleic acid and carrier nucleic acids, wherein target nucleic acid comprises an adaptor capable of hybridizing to a primer; (b) obtaining a substrate comprising the primer; (c) amplifying the target nucleic acid on the substrate, comprising: (i) hybridizing the target nucleic acid to the primer, and (ii) extending the primer; and (d) sequencing the amplified target nucleic acid.

In some embodiments, the target nucleic acid has a concentration less than 10 nM, 100 pM, 20 pM or 5 pM. In some embodiments, step (a) lacks adjusting the concentration of the target nucleic acid.

In some embodiments, the target nucleic acid comprises a single adaptor. In some embodiments, the adaptor comprises a nucleotide sequence selected from a P5 sequence (SEQ ID NO:05), a P7 sequence (SEQ ID NO:06), or a complement thereof.

In some embodiments, the target nucleic acid is (i) derived from a bacteriophage genome. In some embodiments, the bacteriophage genome is a PhiX genome; or (ii) is mammalian; optionally. In some embodiments, the target nucleic acid is human. In some embodiments, the target nucleic acid comprises DNA. In some embodiments, the target nucleic acid is single-stranded.

In some embodiments, the carrier nucleic acids lack the adaptor. In some embodiments, the carrier nucleic acids and the target nucleic acid are each derived from a genome of an organism of a different kingdom, phylum, class, order, family, genus or species. In some embodiments, the carrier nucleic acids are derived from a genome of a fish; optionally. In some embodiments, the carrier nucleic acids comprise salmon sperm DNA, RNA, or siRNA. In some embodiments, the carrier nucleic acids comprise DNA. In some embodiments, the carrier nucleic acids comprise RNA.

In some embodiments, the carrier nucleic acids comprise single-stranded nucleic acids.

In some embodiments, the carrier nucleic acids comprise double-stranded nucleic acids.

In some embodiments, the carrier nucleic acids have an average length less than 5000 consecutive nucleotides. In some embodiments, the carrier nucleic acids have an average length in a range from 100 to 5000 consecutive nucleotides. In some embodiments, the carrier nucleic acids have an average length in a range from 100 to 1000 consecutive nucleotides. In some embodiments, the carrier nucleic acids have an average length greater than 5000 consecutive nucleotides. In some embodiments, the carrier nucleic acids have an average length in a range from 5000 to 10000 consecutive nucleotides.

In some embodiments, the amplifying comprises bridge amplification. In some embodiments, the amplifying comprises exclusion amplification. In some embodiments, the exclusion amplification comprises a reagent selected from a polymerase, such as BSU polymerase; a recombinase, such as UvsX recombinase; a single-stranded DNA binding protein, such as GP32; a crowding agent, such as PEG 6000; and/or creatine phosphate (CP). In some embodiments, the amplifying comprises isothermal amplification.

In some embodiments, the substrate comprises a patterned surface. In some embodiments, the patterned surface comprises a plurality of nanowells. In some embodiments, the substrate comprises a flow cell.

Some embodiments also include denaturing the sample prior to the amplifying. In some embodiments, the denaturing comprises heating the sample, or contacting the sample with NaOH.

### BRIEF DESCRIPTION OF THE DRAWINGS

FIG. 1 depicts an example embodiment of a workflow which includes: fragmenting long input DNA by high molecular weight (HMW) fragmentation and adding adapters, such as by tagmentation using low-density bead-linked transposomes (BLTs); long-range PCR mutagenesis to introduce a signature into long fragments; further library preparation steps, such as additional tagmentation to obtain small fragments with adapters; sequencing and assembly of sequencing reads.

FIG. 2 depicts an example embodiment of a workflow which includes a long-read (‘iLR’, or ‘ILR’) pathway, and a reference pathway. The long-read pathway includes steps for: tagmentation; mutagenesis; bottleneck (suppression) PCR. Both the long-read pathway and reference pathway share steps including: standard library preparation, such as tagmentation; sequencing; and assembly of sequencing reads.

FIG. 3A is a line graph which relates to data acquired from a purified bottlenecking PCR product run on an Agilent Bioanalyzer using a High Sensitivity DNA Kit.

FIG. 3B is a line graph which relates to data acquired from a purified final library prep product run on an Agilent Bioanalyzer using a High Sensitivity DNA Kit.

FIG. 4A depicts line graphs of results of experiments using transposomes in solution at various concentration (left panel); and using BLTs (right panel).

FIG. 4B depicts graphs for a Staphylococcus aureus 4 Mb genome view, with samples at 4 million reads.

FIG. 5 depicts a schematic for workflow steps including High Molecular Weight (HMW) fragmentation; and mutagenesis and suppression PCR in which smaller products form bairpins.

FIG. 6 depicts graphs related to activity and fragment length, including: left panel is a point graph of actual activity units (AU)/μl and median actual AU/μl versus build AU/μl for soluble transposomes (TSM) and BLTs having various densities/activities of transposomes: BLT at low density (BLT-LR) at 0.075 AU/μl, and TDER-BLR comprising A14 and B15 TSMs at 0.1 AU/μl, 0.2 AU/μl, and 0.5 AU/μl. The right upper panel is a line graph of fragment size. The right lower panel is a graph for average size for soluble TSM, and BLTs containing A14 and B15 TSMs, or B15 TSM only.

FIG. 7 depicts graphs related to mutagenesis PCR for soluble TSM, and BLTs containing A14 and B15 TSMs, or B15 TSM only, including: left panel is a point graph of mean yield (ng/μl); right upper panel is a line graph for average size; and right lower panel is a graph for mean average size.

FIG. 8 depicts graphs related to bottleneck (suppression) PCR for soluble TSM, and BLTs containing A14 and B15 TSMs, or B15 TSM only, including: left panel is a point graph of mean yield (ng/μl); right upper panel is a line graph for average size; and right lower panel is a graph for mean average size.

FIG. 9 depicts a point graph for a sequencing metric (GC coverage) for soluble TSM, and BLTs containing A14 and B15 TSMs, or B15 TSM only.

FIG. 10 depicts graphs for a sequencing metric (N50, left panel; and N50by regions, right panel) for soluble TSM, and BLTs containing A14 and B15 TSMs, or B15TSM only. N50 is the length of the shortest contig for which longer and equal length contigs cover at least 50% of the assembly.

FIG. 11 depicts graphs for a sequencing metric (fraction of bases with no coverage, left panel; and fraction of bases with <10× coverage, right panel) for soluble TSM, and BLTs containing A14 and B15 TSMs, or B15 TSM only.

FIG. 12 depict line graphs of various BLT activities (Build AU/μl), and product average size (lower panel), total yield (middle panel), or fluorescent resonance energy transfer (FRET) (upper panel).

FIG. 13 depicts line graphs of various BLT activities (Build AU/μl), and sequencing metrics including SLR coverage depth (lower panels), total bases (middle panels), or N50 (upper panels).

FIG. 14 depicts line graphs of various BLT activities (Build AU/μl), and sequencing metrics including percent duplicated reads (lower panels), fraction of bases with <10× coverage (middle panels), or fraction of bases with no coverage (upper panels).

FIG. 15 depicts line graphs of various BLT activities (AU/μl), and sequencing metrics including SLR coverage depth (lower panel), total bases (lower middle panel), redundancy (upper middle panel), or N50 (upper panel) with three different operators.

FIG. 16 depicts line graphs of tagmentation yield (left panel) or tagmentation fragment length (right panel) for various amounts of input DNA.

FIG. 17 depicts line graphs for various amounts of input DNA and mutagenesis yield (upper left panel), bottleneck yield (middle left panel), library yield (lower left panel), mutagenesis fragment length (upper right panel), bottleneck fragment length (middle right panel), and library fragment length (lower right panel).

FIG. 18 depicts line graphs for various amounts of input DNA and sequencing metrics including: total bases (upper left panel), insert size (middle left panel), percent duplicated reads (lower left panel), total bases (upper right panel), insert size (middle right panel), and library fragment length (lower right panel). The right panels show the same data as the left panels, but without the 1000 ng data point.

FIG. 19 depicts line graphs for various amounts of input DNA and sequencing metrics including: number of MQ0 reads (upper left panel), error rate (upper middle left panel), redundancy (lower middle left panel), N50 (lower left panel), number of MQ0 reads (upper right panel), error rate (upper middle right panel), redundancy (lower middle right panel), N50 (lower right panel). The right panels show the same data as the left panels, but without the 1000 ng data point.

FIG. 20 depicts line graphs for various amounts of input DNA and sequencing metrics including: mode coverage (upper left panel), fraction of bases with no coverage (middle left panel), fraction of bases with <10× coverage (lower left panel), mode coverage (upper right panel), fraction of bases with no coverage (middle right panel), fraction of bases with <10× coverage (lower right panel). The right panels show the same data as the left panels, but without the 1000 ng data point.

FIG. 21 depicts a graph for various amounts of input DNA and sequencing metric (GC bias).

FIG. 22 depicts line graphs for various input DNAs subjected to shearing for different periods of time, control input DNA, and HMW input DNA, and fragment size.

FIG. 23 depicts graphs for various input DNAs subjected to shearing for different periods of time, control input DNA, and HMW input DNA, and tagmentation yield (left panel) or tagmentation fragment length (right panel).

FIG. 24A depicts graphs for various input DNAs subjected to shearing for different periods of time, control input DNA, and HMW input DNA, and mutagenesis yield (left panel) or normalization yield (right panel).

FIG. 24B depicts graphs for various input DNAs subjected to shearing for different periods of time, control input DNA, and HMW input DNA, and bottleneck PCR yield (left panel) or post-bottleneck fragment length (right panel).

FIG. 25 depicts line graphs for various input DNAs subjected to shearing for different periods of time, and HMW input DNA, and sequencing metrics: N50 (left panels) or redundancy (right panels).

FIG. 26 depicts line graphs for various input DNAs subjected to shearing for different periods of time, and HMW input DNA, and sequencing metrics: SLR coverage (upper left panel), fraction with no coverage (middle left panel), fraction with <10× coverage (lower left panel), insert size (upper right panel), percent duplicated reads (upper middle right panel), insertion per 100 kb (lower middle right panel), or MQ0 (lower right panel).

FIG. 27 depicts line graphs for various input DNAs subjected to shearing for different periods of time, and HMW input DNA, and a sequencing metric (GC bias).

FIG. 28 depicts an example overview for enrichment of ‘long fragments’ or ‘short fragments’ in a workflow.

FIG. 29 depicts an example timeline for enrichment of ‘long fragments’ or ‘short fragments’ in a workflow.

FIG. 30 depicts selection of selection probes with higher specificity.

FIG. 31 depicts coverage of selection probes.

FIG. 32A depicts a graph of percentage non-specific genomic DNA binding to beads blocked with various amounts of a 40-mer S-oligo or 40-mer P-oligo (40mer 3-blocked).

FIG. 32B depicts a graph of percentage non-specific genomic DNA binding to beads blocked with various amounts of a 60-mer S-oligo (phosphorothioate-60mer; line running at bottom of graph) or 60-mer P-oligo (phosphate-60mer; line running at top of graph), The ‘good lot’ and ‘bad lot’ were untreated (not blocked) bead lots that had been determined to have relatively low levels or high levels of non-specific DNA binding, respectively, and as shown in the graph.

FIG. 33 depicts a line graph of percentage non-specific genomic DNA binding to beads blocked overnight at 4° C. with various amounts of either a 20-mer S-oligo (phosphorothioate-20mer), a 40-mer S-oligo (phosphorothioate-40mer), or 60-mer S-oligo (phosphorothioate-60mer). The ‘good lot’ and ‘bad lot’ were untreated (not blocked) bead lots that had been determined to have relatively low levels or high levels of non-specific DNA binding, respectively, and as shown in the graph.

FIG. 34 depicts a graph of percentage non-specific genomic DNA binding to beads blocked with 40-mer S-oligo or 40-mer P-oligo overnight at either 4° C. or room temperature. The ‘good lot’ and ‘bad lot’ were untreated (not blocked) bead lots that had been determined to have relatively low levels or high levels of non-specific DNA binding, respectively, and as shown in the graph.

FIG. 35 depicts a graph of percentage non-specific genomic DNA binding to beads blocked with 60-mer S-oligo that had been either (i) washed then blocked, or (ii) blocked then washed, before determining the percentage non-specific DNA binding to the beads. The ‘good lot’ and ‘bad lot’ were untreated (not blocked) bead lots that had been determined to have relatively low levels or high levels of non-specific DNA binding, respectively, and as shown in the graph.

FIG. 36 depicts a graph of percentage non-specific genomic DNA binding to unblocked beads or to beads blocked with 40-mer S-oligo, for various bead lots that had been determined to have relatively low levels of non-specific DNA binding.

FIG. 37 depicts a graph of percentage non-specific genomic DNA binding to unblocked beads or to beads blocked with 40-mer S-oligo for various bead lots. Blocked beads were treated with 1000 ng/100 μl 40-mer S-oligo.

FIG. 38 depicts a graph of percentage non-specific genomic DNA binding to beads and amount of blocker (40-mer S-oligo) added to the beads for various bead lots. The ‘good’ control was an untreated (not blocked) bead lot that had been determined to have relatively low levels of non-specific DNA binding.

FIG. 39 depicts bar graphs for sequencing metrics including N50,redundancy, and percentage duplicated reads with regard to sequences within four different genomic regions, for a workflow that included the use of blocked or unblocked beads from various bead lots.

FIG. 40A depicts bar graphs for sequencing metrics including fraction of bases with no coverage, fraction of bases with coverage <10, and number of MQ0 reads with regard to sequences within four different genomic regions, for a workflow that included the use of blocked or unblocked beads from various bead lots.

FIG. 40B depicts bar graphs for sequencing metrics including mode coverage, total bases, and error rate with regard to sequences within four different genomic regions, for a workflow that included the use of blocked or unblocked beads from various bead lots.

FIG. 41 depicts a line graph of normalized coverage and percentage GC bias in a workflow that included either blocked or unblocked beads for various beads lots. The solid line depicts an average for blocked beads, and the dotted line depicts an average for beads that had not been blocked.

FIG. 42 depicts a bar graph with error bars of percentage non-specific genomic DNA binding to beads blocked with various oligonucleotides containing different numbers of phosphate substitutions in the oligonucleotide backbone. Within the graph are representations of the oligonucleotides showing regions including the phosphate substitutions. The ‘good control’ and ‘bad control’ were untreated (not blocked) bead lots that had been determined to have relatively low levels or high levels of non-specific DNA binding, respectively.

FIG. 43 depicts a graph of average cluster density for 1 nM or 100 pM PhiX stock solutions that lacked a S-oligo 60mer and underwent freeze-thaw cycles (force fail control); included a S-oligo 60mer and underwent freeze-thaw cycles (force fail S-oligo 60mer); did not undergo frecze-thaw cycles (no force fail, control).

FIG. 44 depicts a graph of cluster density for samples run on a sequencing platform that had been incubated at room temperature for various periods of time.

FIG. 45 depicts a graph of a percentage pass filter (% PF) for samples run on a sequencing platform that had undergone various numbers of cycles of freezing and thawing.

FIG. 46A, FIG. 46B and FIG. 46C depict example plots for sequencing runs performed on a patterned in which samples were unloaded (FIG. 46A), overloaded (FIG. 46B), or optimally loaded (FIG. 46C).

FIG. 47A, FIG. 47B and FIG. 47C depict three plots for sequencing runs performed on an ISEQ™ (Illumina, Inc., San Diego) sequencing platform with control samples that included fresh 60 pM PhiX; and test samples which included 60 pM PhiX and carrier DNA incubated at room temperature for 24 hours.

FIG. 48 depicts an additional two plots for sequencing runs with control samples that included fresh 200 pM PhiX; and test samples which included 200 pM PhiX and carrier DNA incubated at room temperature for 24 hours.

FIG. 49 depicts a graph of average cluster density for 10 nM, 1 nM or 100pM PhiX stock solutions that included carrier DNA (carrier DNA) or lacked carrier DNA (control), and underwent freeze-thaw cycles.

### DETAILED DESCRIPTION

Some embodiments of the methods and compositions provided herein relate to blocked substrates in which non-specific binding of nucleic acids to the blocked substrate is reduced. Some such embodiments include beads contacted with an oligonucleotide, such as an oligonucleotide containing one or more phosphorothioate bonds, wherein the oligonucleotides reduce non-specific nucleic acid binding to the beads. Such substrates are useful in methods for obtaining long-read information from short reads of a target nucleic acid. Some embodiments include the use of carrier nucleic acids.

Some embodiments of the methods and compositions provided herein include use of carrier nucleic acids to maintain the activity of certain nucleic acid reagents, such as nucleic acid reagents useful in certain sequencing systems. For example, reagents such as control nucleic acid samples useful to validate software and hardware associated with certain sequencing platforms. Some embodiments include methods of sequencing a target nucleic acid, such as a control nucleic acid. In some embodiments, the method includes (a) obtaining a sample comprising the target nucleic acid and carrier nucleic acids, wherein target nucleic acid comprises an adaptor capable of hybridizing to a primer; (b) obtaining a substrate comprising the primer; (c) amplifying the target nucleic acid on the substrate, comprising: (i) hybridizing the target nucleic acid to the primer, and (ii) extending the primer; and (d) sequencing the amplified target nucleic acid. In some embodiments, the sample comprises a plurality of target nucleic acids, wherein each target nucleic acid comprises the adaptor.

Some embodiments of the methods and compositions provided herein relate to obtaining long-read information from short reads of a target nucleic acid. In some such embodiments, a workflow can include initial steps to selectively generate, amplify, and mark long nucleic acid fragments. Further steps can include fragmenting the long nucleic acid fragments into shorter fragments for sequencing and using computer systems with processors to informatically reconstruct a nucleotide sequence of the target nucleic acid. Some such embodiments include the use of blocked substrates and/or carrier nucleic acids.

Prior fragmentation methods typically generated a very wide distribution of fragment sizes such that even when aiming for large fragments, inevitably short fragments were included. Such short fragments are ‘wasted’ space, giving very little new information. Some embodiments provided herein preserve long (2,000-40,000 bp) fragments, mark them, and carry them through into a short-read portion of a workflow so they can then be reconstructed into their parent long fragments informatically. Shorter fragments are much less desirable and will take up valuable sequencing space and informatics volume if they are included.

In prior short-read library preps, most size selection is done by a combination of (1) initial fragmentation and (2) solid-phase reversible immobilization (SPRI)-based size selection. However, SPRI-based size selection primarily works on fragments smaller than about 1000 bp in length. In contrast, suppression (‘bottlenecking’ or ‘bottleneck’) PCR acts on larger fragments. Suppression PCR entails appending complementary sequences on 5′ and 3′ ends of the same DNA molecule, such that during a PCR annealing step, there is a direct competition between annealing of a primer and annealing of opposite ends of the same DNA fragment. When the PCR primer anneals, extension proceeds as normal, and the fragment is amplified. When opposite ends anneal, for example by forming a hairpin, there is no templated 3′ hydroxyl to extend, and so amplification does not occur. A key to suppression PCR and size selection is that for shorter fragments, the opposite ends of the same fragment are closer together and therefore more likely to find each other and anneal. Under optimized conditions, this leads to preferential amplification of longer fragments. Aspects of suppression PCR useful with embodiments provided herein are described in Dai, Z-M, et al (2006) J. of Biotech 128:435-443; and Rand K. N. et al., (2005) N.A. Res. 33:e127 which are incorporated by reference in their entireties.

In some embodiments provided herein, complementary 5′ and 3′ ends are achieved by an initial tagmentation step with B15 transposomes only. Typically, tagmentation would be performed with a combination of A14 and B15 transposomes. so that the different sequences can be used for read 1 and read 2 primers during subsequent sequencing. However, because the initial tagmentation in certain embodiments provided herein is used to provide a landing spot for PCR, different sequences for read 1 and read 2 primers do not need to be added at this stage. In contrast to SPRI-based size selection, it was observed that by adding cycles of suppression PCR, the number of fragments under 2000 bp in length can be dramatically reduced.

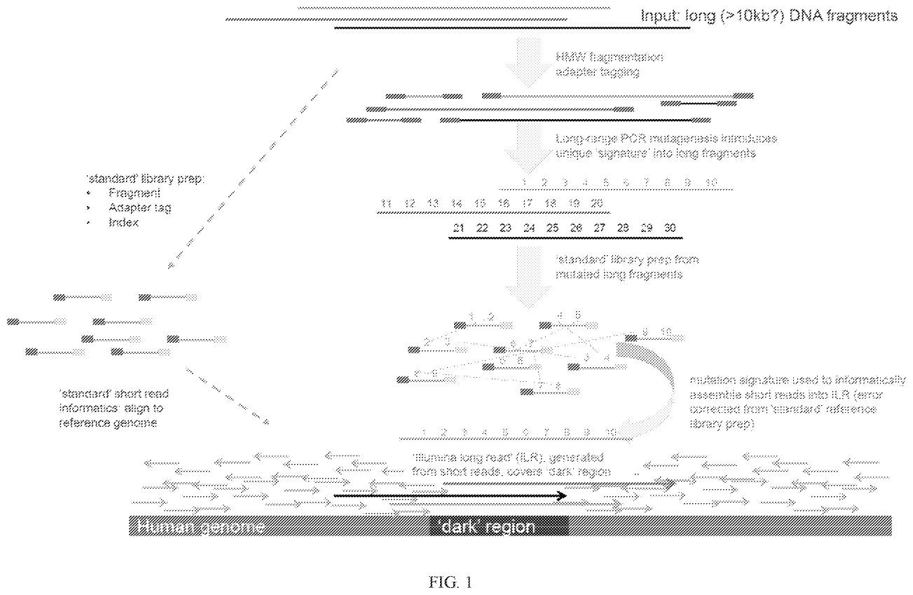

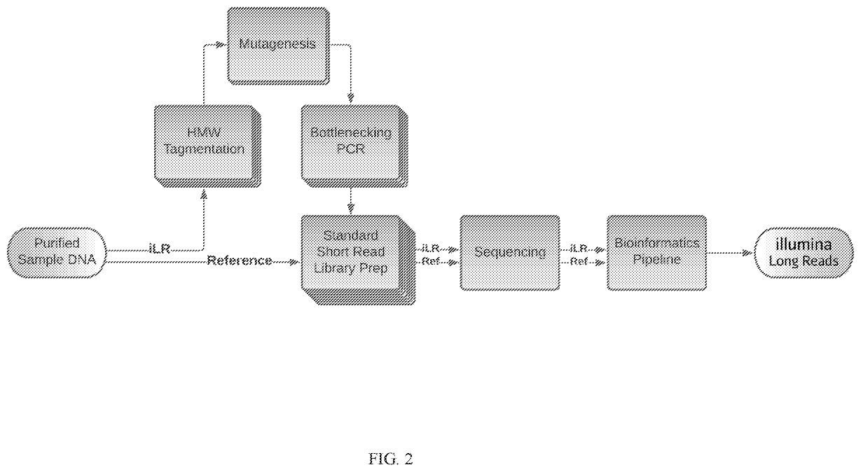

In some embodiments provided herein a workflow includes: fragmenting long input DNA by high molecular weight (HMW) fragmentation and adding adapters, such as by tagmentation using low-density bead-linked transposomes (BLTs); long-range PCR mutagenesis to introduce a signature into long fragments; further library preparation steps, such as additional tagmentation to obtain small fragments with adapters; sequencing and assembly of sequencing reads (FIG. 1). In some embodiments provided herein a workflow includes a long-read (iLR) pathway, and a reference pathway. The long-read pathway includes steps for: tagmentation; mutagenesis; bottleneck (suppression) PCR. Both the long-read pathway and reference pathway share steps include: standard library preparation, such as tagmentation; sequencing; and assembly of sequencing reads (FIG. 2).

Certain aspects useful with embodiments of the methods and compositions provided herein are disclosed in U.S. Pat. Nos. 9,040,256; 9,683,230; U.S. 2021/0010008; PCT/US2023/067467; PCT/US2023/067465; PCT/US2023/067466; PCT/US2023/067471; and PCT/US2023/067468 which are each incorporated by reference in its entirety.

### Definitions

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably. Some embodiments provided herein include nucleic acids comprising a backbone in which the backbone includes phosphodiester bonds, such that a bond between bases includes: —O—P(═O)(O)—O—. In some embodiments, the backbone includes phosphorothioate bonds, such as —O—P(═S)(O)—O—.

As used herein “transposome” includes a complex comprising of at least one transposase enzyme and a transposon recognition sequence, such as a transposon adapter. In some such systems, the transposase binds to a transposon recognition sequence to form a functional complex that is capable of catalyzing a transposition reaction. In some aspects, the transposon recognition sequence is a double-stranded transposon end sequence. The transposase, or integrase, binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting also in a cleavage event. Exemplary transposition procedures and systems that can be readily adapted for use with the transposases of the present disclosure are described, for example, in WO10/048605, U.S. 2012/0301925, U.S. 2012/13470087, or U.S. 2013/0143774, each of which is incorporated herein by reference in its entirety.

In some embodiments, the transposome complex is a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are heterodimers dimers, wherein the first population has a first adaptor sequence in each monomer and the second population has a different adaptor sequence in each monomer.

As used herein “solid surface,” “solid support,” and other grammatical equivalents refer to any material that is appropriate for or can be modified to be appropriate for the attachment of the transposome complexes. As will be appreciated by those in the art, the number of possible substrates is multitude. Possible substrates include, but are not limited to, glass and modified or functionalized glass, plastics (including acrylics, polystyrene and copolymers of styrene and other materials, polypropylene, polyethylene, polybutylene, polyurethanes, TEFLON, etc.), polysaccharides, nylon or nitrocellulose, ceramics, resins, silica or silica-based materials including silicon and modified silicon, carbon, metals, inorganic glasses, plastics, optical fiber bundles, beads, paramagnetic beads, and a variety of other polymers. In some such embodiments, the transposome complex is immobilized on the solid support via the linker. In some further embodiments, the solid support comprises or is a tube, a well of a plate, a slide, a bead, or a flowcell, or a combination thereof. In some further embodiment, the solid support comprises or is a bead. In one embodiment, the bead is a paramagnetic bead. In some of the methods and compositions presented herein, transposome complexes are immobilized to a solid support. In one embodiment, the solid support is a bead. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and TEFLON, as well as any other materials outlined herein for solid supports.

As used herein, “tagmentation: includes to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those of skill in the art.

### Certain Methods for Preparing Nucleic Acid Libraries

Some embodiments of the methods and compositions providing herein include preparing a nucleic acid library. Some such embodiments include (a) obtaining a plurality of transposomes comprising transposon adaptors, wherein the plurality of transposomes is immobilized on a solid support; (b) contacting a plurality of nucleic acid fragments with the plurality of transposomes to obtain a plurality of polynucleotides; (c) amplifying the plurality of polynucleotides to obtain amplified polynucleotides; and (d) adding library adapters to each end of the amplified polynucleotides, thereby obtaining the nucleic acid library. In some embodiments, an amount of the plurality of nucleic acid fragments is less than about 100 ng, 50 ng, 30 ng, 20 ng, 10 ng, 5 ng, or 1 ng.

Some embodiments include an initial tagmentation step which fragments the plurality of nucleic acids fragments and adds an adaptor to each end of the products of the tagmentation. The initial tagmentation is limited such that the products of the tagmentation are longer than a tagmentation where the activity of transposomes is not limited.

Certain aspects useful with embodiments of the methods and compositions provided herein are disclosed in U.S. Pat. Nos. 9,115,396; 9,080,211; 9,040,256; U.S. 2014/0194324, each of which is incorporated herein by reference in its entirety.

In some embodiments, the solid support comprises a bead, such as a blocked bead described herein. In some such embodiments, the transposomes are bead-linked transposomes (BLTs). In some embodiments, the activity of the transposomes on the beads is such that a tagmentation reaction with the BLTs and the plurality of nucleic acid fragments results in long polynucleotides, such as polynucleotides an having average length of the plurality of polynucleotides greater than about 1 kb, 2 kbp, 5 kbp, 10 kbp, 15 kbp, 20 kbp, or 40 kbp. For example, the transposomes can be bound at a low density on the beads; and/or have a low tagmentation activity. In some embodiments, the number of transposomes immobilized on the bead is no more than about 100 transposomes, 50 transposomes, 40 transposomes, 30 transposomes, 20 transposomes, or 10 transposomes. In some embodiments, the number of transposomes immobilized on the bead is no more than about 30 transposomes. In some embodiments, the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp. In some embodiments, the plurality of the transposomes immobilized on the bead comprise a tagmentation activity in a range from about 0.05 AU/μl to about 0.25 AU/μl. In some embodiments, the plurality of the transposomes immobilized on the bead comprise a tagmentation activity of about 0.075 AU/μl.

In some embodiments, the transposomes on the beads are the same. For example, in some embodiments, the transposon adapters comprise the same sequence. In some embodiments, the transposomes of the plurality of transposomes are B15 transposomes. In some embodiments, the transposon adapters comprise the nucleotide sequence: SEQ ID NO:01 (GTCTCGTGGGCTCGG), or a nucleotide sequence having at least 70%, 75%, 80%, 85%, 90%, 95%, or 100% sequence identity to SEQ ID NO:01.

Some embodiments also include steps to add a signature to the products of the initial tagmentation. For example, a signature can be added into the sequence of the library products by steps that include limited mutagenesis. In some embodiments, step (c) comprises a mutagenesis PCR, such that mutations are introduced into amplified polynucleotides. In some embodiments, the mutagenesis PCR comprises amplifying the plurality of polynucleotides with a low-bias DNA polymerase, and/or with a nucleotide analogue. In some embodiments, the nucleotide analogue comprises dPTP, and/or 8-oxo-dGTP. dP contains the bicyclic pyrimidine analog 3,4-dihydro-8H-pyrimido-[4,5-C][1,2]oxazin-7-one. In some embodiments, the low-bias DNA polymerase is a Thermococcal polymerase, or a functional derivative thereof. In some embodiments, the Thermococcal polymerase is derived from a Thermococcal strain selected from the group consisting of T. kodakarensis, T. siculi, T. celer and T. sp KS-1. In some embodiments, the mutagenesis PCR comprises no more than 12 cycles, 10 cycles, 9 cycles, 8 cycles, 7 cycles, 6 cycles, 5 cycles, 4 cycles, 3 cycles, or 2 cycles, In some embodiments, the mutagenesis PCR comprises no more than 6 cycles.

Some embodiments also include a bottleneck or suppression PCR step to enrich for longer polynucleotides. For example, shorter amplified polynucleotides form hairpins, while longer amplified polynucleotides may be further amplified. Some such embodiments can enrich for longer fragments. In some such embodiments, a first end of a polynucleotide of the plurality of polynucleotides is capable of annealing to a second end of the polynucleotide of the plurality of polynucleotides; and/or, wherein a first end of an amplified polynucleotide is capable of annealing to a second end of the amplified polynucleotide. In some embodiments, the suppression PCR comprises use of a single amplification primer. In some embodiments, the amplified polynucleotides have an average length greater than about 1 kbp, 2 kbp, 3 kbp, 4 kbp, 5 kbp, 10 kbp, 15 kbp, or 20 kbp. In some embodiments, the suppression PCR comprises no more than 16 cycles, 14 cycles, 10 cycles, 9 cycles, 8 cycles, 7 cycles, 6 cycles, 5 cycles, 4 cycles, 3 cycles, or 2 cycles. In some embodiments, the suppression PCR comprises no more than 6 cycles.

Detailed descriptions of certain embodiments of suppression PCR are found in, e.g., U.S. Pat. No. 5,565,340 and Siebert et al., Nucleic Acids Res., 23(6):1087-1088 (1995). Briefly, the inverted repeat sequences function as suppression tails by competing with the suppression PCR primer for complementary binding. The inverted repeats tend to anneal each other, thereby preventing PCR primer binding. Since shorter amplicons undergo inverted repeat annealing more often than longer amplicons, the suppression PCR favors generating long amplicons.

Some embodiments also include enriching for target nucleic acids in the amplified polynucleotides, such as products of the suppression PCR. In some embodiments, the enriching comprises hybridizing a plurality of selection probes with the amplified polynucleotides, wherein the plurality of selection probes is capable of specifically hybridizing with the target nucleic acids. In some embodiments, the plurality of selection probes lacks sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon. Some embodiments also include amplifying the target nucleic acids.

Some embodiments also include preparing a library of shorter fragments from the products of the suppression PCR. For example, the products of the suppression PCR can undergo an additional tagmentation. In some embodiments, step (d) comprises contacting the amplified polynucleotides with an additional plurality of transposomes. In some embodiments, the additional plurality of transposomes comprise transposon adapters comprising (i) indexes, and/or (iii) sequencing primer binding sites.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Example selection probes are disclosed in PCT/US2023/067467; PCT/US2023/067465; PCT/US2023/067466; PCT/US2023/067471; and PCT/US2023/067468 which are each incorporated by reference in its entirety. Some embodiments also include amplifying the target polynucleotides.

Some embodiments include methods for preparing a nucleic acid library, comprising: (a) obtaining a plurality of transposomes comprising transposon adaptors, wherein the plurality of transposomes is immobilized on a bead, such as a blocked bead described herein, and wherein the transposomes of the plurality of transposomes are the same; (b) contacting a plurality of nucleic acid fragments with the plurality of transposomes to obtain a plurality of polynucleotides, wherein the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; (c) amplifying the plurality of polynucleotides to obtain amplified polynucleotides by: (i) performing a mutagenesis PCR, such that mutations are introduced into amplified polynucleotides, and (ii) performing a suppression PCR; and (d) adding library adapters to each end of the amplified polynucleotides by contacting the amplified polynucleotides with an additional plurality of transposomes, thereby obtaining the nucleic acid library.

Some embodiments also include enriching for target nucleic acids in the amplified polynucleotides. In some embodiments, the enriching comprises hybridizing a plurality of selection probes with the amplified polynucleotides, wherein the plurality of selection probes is capable of specifically hybridizing with the target nucleic acids. In some embodiments, the plurality of selection probes lack sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon. Some embodiments also include amplifying the target nucleic acids.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Example selection probes are disclosed in PCT/US2023/067467; PCT/US2023/067465; PCT/US2023/067466; PCT/US2023/067471; PCT/US2023/067468 which are each incorporated by reference in its entirety. Some embodiments also include amplifying the target polynucleotides.

Some embodiments also include methods for determining a sequence of a target nucleic acid, comprising preparing a nucleic acid library by any one of the embodiment above, sequencing the library of nucleic acids to obtain sequence reads; and assembling sequence reads to obtain the sequence of a target nucleic acid. In some embodiments, the assembling comprises comparing the sequence reads to a reference sequence. In some embodiments, the reference sequence is obtained from the same nucleic acid sample as the plurality of nucleic acid fragments.

### Blocked Substrates

Some embodiments of the methods and compositions provided herein include blocked substrates. Blocking reduces non-specific binding of nucleic acids to the substrate. In some embodiments, an oligonucleotide is used to block sites available for non-specific binding of nucleic acids to a substrate.

Some embodiments include blocked substrates comprising a plurality of beads. In some embodiments, the plurality of beads comprise a blocked bead, such as a magnetic bead in contact with an oligonucleotide, wherein the oligonucleotide is configured to block binding to the bead, such as non-specific binding of nucleic acids to the bead. In some embodiments, the oligonucleotide comprises a backbone comprising a phosphorothioate bond. In some such embodiments, non-specific nucleic acid binding to the blocked bead is reduced compared to a bead not contacted with the oligonucleotide.

In some embodiments, an agent is bound to a surface of the bead, wherein the agent is selected from streptavidin, biotin, or a derivative thereof.

In some embodiments, the nucleic acid comprises DNA. In some embodiments, the nucleic acid comprises genomic DNA.

In some embodiments, the oligonucleotide comprises at least 20, 30, 40, 50, 60, 80, 100 consecutive nucleotides or any number of consecutive nucleotides between any of the foregoing numbers. In some embodiments, the oligonucleotide has a length in a range from 10-100 consecutive nucleotides, 20-80 consecutive nucleotides, 40-80 consecutive nucleotides, 50-80 consecutive nucleotides, or 55-65 consecutive nucleotides.

In some embodiments, at least 40%, 50%, 60%, 70%, 80%, 90%, or 100% of the backbone comprises phosphorothioate bonds. As will be understood, the sugar moieties in native-state oligonucleotides are linked via phosphate (includes a non-bridging oxygen) whereas the sugar moieties in oligonucleotides modified with sulfurizing reagent are linked by phosphorothioate (includes a non-bridging sulfur). Each phosphorothioate can exist as a diastereomer as shown in the example structures below which show a stereogenic a- phosphorus at one internucleotide linkage. The random R and S configuration can result in 2 diastereomers at every single phosphorothioate backbone. Thus, oligonucleotides having an increasing number of phosphorothioate bonds will include an increasing number of different structural isomers. Without being bound to any one theory, this number of different structural isomers is 2ⁿ where n is the number of phosphorothioate sugar backbones. These structural isomers may have a non-linear shape and usually more rigid, the more phosphorothioate bonds, the more rigid for the structural isomers, the end result can be the structural isomers may have many different 3-D structures like many 3-D origamis, drastically different from linear flexible noodle like structures that normal phosphate backbones DNA may present. These many 3-D origami structures may contribute to the following: First, in terms of stabilizing Phix concentrations, the exponential amount of 3-D origami structures may bind onto rough plastic container surface wherever they fit and make the surface less rough, thus to reduce Phix sample loss on the surface. Second, in terms of reducing streptavidin magnetic beads surface non-specific binding of DNA library samples, the exponential amount of 3-D origami structures may be one of the contribution factors by binding onto rough streptavidin magnetic beads surface wherever they fit and make the surface less rough, thus to reduce DNA library samples non-specific binding.

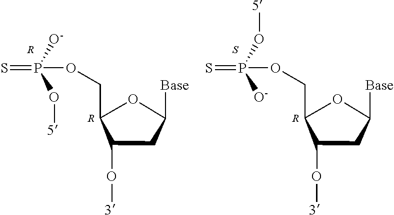

In some embodiments, the oligonucleotide comprises a sequence lacking the capability of forming certain secondary structures, such as a hairpin structure, or other double-stranded structures. Sequences can be developed with software to predict sequences unlikely to form secondary structures, such as a hairpin structure at certain temperatures. In some embodiments, the oligonucleotide comprises a sequence predicted to lack the capability of forming a hairpin structure at a temperature less than 50° C., 30° C., 25° C., 20° C., 15° C., 10° C., 5° C., 0° C., −10° C., −20° C., −30° C., or −40° C. In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO: 04. In some embodiments, the oligonucleotide comprises the nucleotide sequence set forth in SEQ ID NO:02.

In some embodiments, the oligonucleotide comprises DNA. In some embodiments, the oligonucleotide comprises RNA. In some embodiments, the oligonucleotide is single-stranded. In some embodiments, the blocked bead also includes a transposome bound to the bead.

### Reducing Non-Specific Nucleic Acid Binding to a Substrate

Some embodiments of the methods and compositions provided herein include methods for reducing non-specific nucleic acid binding to a substrate, and/or for preparing a substrate in which non-specific nucleic acid binding to the substrate is reduced. Some such embodiments include contacting the substrate with an oligonucleotide, wherein the oligonucleotide comprises a backbone comprising a phosphorothioate bond, and wherein non-specific nucleic acid binding to a substrate is reduced compared to a substrate not contacted with the oligonucleotide.

In some embodiments, the contacting is for a period greater than 30 minutes, greater than 1 hour, greater than 6 hours, or greater than 12 hours.

In some embodiments, the contacting is performed at room temperature. In some embodiments, the contacting is performed at about 4° C.

Some embodiments also include contacting the substrate with a plurality of transposomes. Some embodiments also include contacting the substrate with genomic DNA.

In some embodiments, the substrate comprises a bead. In some embodiments, the substrate comprises a magnetic bead. In some embodiments, an agent is bound to a surface of the substrate, wherein the agent is selected from streptavidin, biotin, or a derivative thereof.

In some embodiments, the nucleic acid comprises DNA. In some embodiments, the nucleic acid comprises genomic DNA.

In some embodiments, the oligonucleotide comprises at least 20, 30, 40, 50, 60, 80, 100 consecutive nucleotides or any number of consecutive nucleotides between any of the foregoing numbers. In some embodiments, the oligonucleotide has a length in a range from 10-100 consecutive nucleotides, 20-80 consecutive nucleotides, 40-80 consecutive nucleotides, 50-80 consecutive nucleotides, or 55-65 consecutive nucleotides.

In some embodiments, at least 40%, 50%, 60%, 70%, 80%, 90%, or 100% of the backbone comprises phosphorothioate bonds.

In some embodiments, the oligonucleotide comprises a sequence lacking the capability of forming certain secondary structures, such as a hairpin structure, or other double-stranded structures. Sequences can be developed with software to predict sequences unlikely to form secondary structures, such as a hairpin structure at certain temperatures. In some embodiments, the oligonucleotide comprises a sequence predicted to lack the capability of forming a hairpin structure at a temperature less than 50° C., 30° C., 25° C., 20° C., 15° C., 10° C., 5° C., 0° C., −10° C., −20° C., −30° C., or −40° C. In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO: 04. In some embodiments, the oligonucleotide comprises the nucleotide sequence set forth in SEQ ID NO:02.

In some embodiments, the oligonucleotide comprises DNA. In some embodiments, the oligonucleotide comprises RNA. In some embodiments, the oligonucleotide is single-stranded.

Some embodiments include normalizing a level of non-specific nucleic acid binding to a plurality of substrates. Some such embodiments can include any one of the foregoing methods. In some embodiments, the plurality of substrates comprises beads from different lots. For example, beads from different lots can include beads obtained from a single manufacturer that have been prepared at different times, or using different equipment, or from different source materials. In some embodiments, beads from different lots can include beads obtained from more than one manufacturer.

Some embodiments of the methods and compositions provided herein include a composition prepared by any one of the foregoing methods. In some such embodiments, the substrate comprises a plurality of beads.

### Carrier Nucleic Acids

Some embodiments of the methods and compositions provided herein include the use of carrier nucleic acids. Carrier nucleic acids can include DNA or RNA, such as salmon sperm DNA, RNA, siRNA, and single-stranded DNA. Carrier nucleic acids can be used to reduce nucleic acid sample losses to tubing and workflow surface exposures during certain methods, such as pre-hybridization and hybridization steps.

Certain sequencing platforms can include clustering protocols in which target nucleic acids may be amplified. Examples of clustering protocols include bridge amplification and exclusion amplification. Bridge amplification can be performed on unpatterned substrates. Exclusion amplification can be performed on patterned substrates, such as substrates comprising a plurality of nanowells. Aspects of exclusion amplification methods, and systems and compositions useful with embodiments provided herein are disclosed in U.S. Pat. No. 8,895,249, which is incorporated by reference herein in its entirety. It was discovered that the presence of carrier nucleic acids did not interfere with amplification methods and clustering protocols.

A correlation between traditional amplification techniques, such as PCR, and clustering protocols was made in an evaluation of FIT sequencing data with greater than 300 runs on patterned flow cells. Specifically, a correlation was determined between primer lawn density, numbers of usable clusters, and signal duration. For example, within a particular linear range, increases in primer density correlated with increases in the brightness of a cluster in certain sequencing protocols and resulted in better signal quality in later cycles. Without being bound to any one theory, it was envisioned that this mechanism was similar to that of PCR on a patterned flow cells, with the number of primers attached to the patterned wells being the driver for final usable molecules per cluster.

As described herein, studies were performed in which carrier DNA was added into sequencing samples at concentrations including 10 pM to 100 pM, depending on the sequencing platform. Carrier DNA was added into all samples and it was found that all current sequencing clustering protocols tolerated carrier DNA and did not bias the clustering when compared to controls without the added carrier DNA.

Another set of experiments described herein was performed in which ˜100 pM carrier DNA was added into samples and incubated for 24 hours at room temperature. In this study, sequencing metrics were substantially similar to those of a control which was loaded directly onto a sequencing platform and not incubated for 24 hours. These experiments revealed that sequencing platforms which include onboard denaturization and offboard denaturization steps could unravel annealing or tangling with the carrier DNA.

Certain embodiments provided herein include reducing variations of nucleic acid reagents, such as nucleic acids in solution, including for example PhiX control nucleic acid samples. For example, statistical data from certain sequencing platforms has shown variation of the PhiX sample was related to a large percentage of the PF losses or variation.

Currently, end users should titrate PhiX control samples to obtain a control sample which will provide appropriate sequencing metrics on a sequencing platform. The difference in the activities of different lots or samples of a nucleic acid reagent, such as PhiX. may be due to differences in packaging, shipping and handling.

Carrier nucleic acids, such as carrier DNA can reduce variation between reagents by buffering environmental conditions and physical effects on target nucleic acids, such as the aforementioned PhiX control nucleic acids. For example, buffering can include reducing contacts of the target nucleic acids with the sides of a vessel, or reducing freeze-thaw effects. In some embodiments, the use of carrier nucleic acids can include shipping samples at an appropriate loading concentration for a sequencing platform, such that an end user may no longer need to titrate a sample.

Some embodiments of the methods and compositions provided herein include use of carrier nucleic acids to maintain the activity of certain nucleic acid reagents, such as nucleic acid reagents useful in certain sequencing systems. For example, reagents such as control nucleic acid samples useful to validate software and hardware associated with certain sequencing platforms.

Some embodiments include methods of sequencing a target nucleic acid, such as a control nucleic acid. In some embodiments, the method includes (a) obtaining a sample comprising the target nucleic acid and carrier nucleic acids, wherein target nucleic acid comprises an adaptor capable of hybridizing to a primer; (b) obtaining a substrate comprising the primer; (c) amplifying the target nucleic acid on the substrate, comprising: (i) hybridizing the target nucleic acid to the primer, and (ii) extending the primer; and (d) sequencing the amplified target nucleic acid. In some embodiments, the sample comprises a plurality of target nucleic acids, wherein each target nucleic acid comprises the adaptor.

In some embodiments, use of carrier nucleic acids were found to buffer environmental or physical effects which may reduce the activity of a nucleic acid reagent, such as activity related to sequencing efficiencies. Buffered nucleic acid reagents may be provided at lower concentrations than unbuffered nucleic acid reagents which lack carrier nucleic acids. In some embodiments, the plurality of target nucleic acids, such as control nucleic acids, has a concentration less than 10 nM, 100 pM, 20 pM, or 5 pM.

In some embodiments, an end-user may not have a need to titrate or readjust the concentration of a nucleic acid reagent because the activity of that reagent may not have been changed, or changed unpredictably, from the activity of the reagent at a source, such as when it was produced and/or the site of its production. In some embodiments, step (a) above may lack adjusting the concentration of the plurality of target nucleic acids.

In some embodiments, the target nucleic acid comprises a nucleic acid library. In some embodiments, such a library can include aspects such as adaptors, read/sequencing primer binding sites, and unique sequences such as barcodes. In some embodiments, the adaptors of the plurality of target nucleic acids are the same as one another. In some embodiments, the adaptor comprises a nucleotide sequence selected from a P5 sequence (AATGATACGGCGACCACCGA) SEQ ID NO: 5, a P7 sequence (CAAGCAGAAGACGGCATACGAGAT) SEQ ID NO: 6, or a complement thereof. In some embodiments, the target nucleic acid is a control for a sequencing platform. In some embodiments, the target nucleic acid is derived from a bacteriophage genome. In some embodiments, the bacteriophage genome is a PhiX genome. In some embodiments, the target nucleic acid is mammalian. In some embodiments, the target nucleic acid is human. In some embodiments, the target nucleic acid comprises DNA. In some embodiments, the target nucleic acid is single-stranded.

As used herein “carrier nucleic acids” can include nucleic acids that buffer a nucleic acid reagent from environmental and physical effects. The carrier nucleic acids can be inert in reactions in which the reagent is a participant. In some embodiments, the carrier nucleic acids lack the adaptor. In some embodiments, the carrier nucleic acids and the target nucleic acid are each derived from a genome of an organism of a different kingdom, phylum, class, order, family, genus or species. In some embodiments, the carrier nucleic acids are derived from a genome of a fish. In some embodiments, the carrier nucleic acids comprise salmon sperm DNA, RNA, or siRNA. More examples of carrier nucleic acids include bacterial nucleic acids, and nucleic acids such as plasmids. In some embodiments, the carrier nucleic acids comprise DNA. In some embodiments, the carrier nucleic acids comprise RNA. In some embodiments, the carrier nucleic acids comprise single-stranded nucleic acids. In some embodiments, the carrier nucleic acids comprise double-stranded nucleic acids. In some embodiments, the carrier nucleic acids have an average length less than 5000 consecutive nucleotides. In some embodiments, the carrier nucleic acids have an average length in a range from 100 to 1000 consecutive nucleotides. In some embodiments, the carrier nucleic acids have an average length in a range from 100 to 5000 consecutive nucleotides. In some embodiments, the carrier nucleic acids have an average length greater than 5000 consecutive nucleotides. In some embodiments, the carrier nucleic acids have an average length in a range from 5000 to 10000 consecutive nucleotides.

In some embodiments, the amplifying comprises bridge amplification. In some embodiments, the amplifying comprises exclusion amplification. Aspects of exclusion amplification methods, systems and compositions useful with embodiments provided herein are disclosed in U.S. Pat. No. 8,895,249 which is incorporated by reference herein in its entirety. In some embodiments, the exclusion amplification comprises a reagent selected from a polymerase, such as BSU polymerase; a recombinase, such as UvsX recombinase; a single-stranded DNA binding protein, such as GP32; a crowding agent, such as PEG 6000; and/or creatine phosphate (CP). In some embodiments, the amplifying comprises isothermal amplification. In some embodiments, the substrate comprises a patterned surface. In some embodiments, the patterned surface comprises a plurality of nanowells. In some embodiments, the substrate comprises a flow cell.

Some embodiments also include denaturing the sample prior to the amplifying. In some embodiments, the denaturing comprises heating the sample, or contacting the sample with NaOH.

### Stabilizing Nucleic Acid Samples

Some embodiments of the methods and compositions provided herein include stabilizing a nucleic acid sample. For example, a nucleic acid sample, such as a sample from an organism, population or individual, or a control sample, such as a sequencing control, such as a bacteriophage genome, such as PhiX control, may be subjected to environmental or temporal factors which can degrade the quality of the nucleic acid. Low concentration samples, such as concentrations less than concentration less than 10 nM, 100 pM, 20 pM, or 5 pM, may be particularly vulnerable to degradation. Degradation can result in reduced ability to analyze, such as sequence, the nucleic acid successfully, and can be measured by reduced quality of sequencing metrics such as N50, GC bias, percentage duplicated reads, redundancy of reads, error rate, CFR intensity, percentage alignment, percentage pass filter, cluster pass filter, and average cluster density. Some embodiments provided herein include the use of oligonucleotides or carrier DNA to stabilize a nucleic acid sample. Some embodiments for stabilizing a nucleic acid sample include contacting the nucleic acid sample with (i) an oligonucleotide, wherein the oligonucleotide comprises a backbone comprising a phosphorothioate bond; or (ii) carrier nucleic acids. Some embodiments also include sequencing the nucleic acid sample, wherein sequence data obtained from the nucleic acid sample is improved compared to a nucleic acid same lacking the oligonucleotide or carrier nucleic acids. In some embodiments, the improvement comprises an improved sequencing metric selected from N50, GC bias, percentage duplicated reads, redundancy of reads, error rate, CFR intensity, percentage alignment, percentage pass filter, cluster pass filter, and average cluster density. In some embodiments, the nucleic sample has concentrations less than concentration less than 500 nM, 100 nM, 10 nM, 100 pM, 20 pM, or 5 pM.

In some embodiments, the oligonucleotide comprises 20, 40, 60 or more consecutive nucleotides. In some embodiments, the oligonucleotide comprises or consists of 60 consecutive nucleotides. In some embodiments, at least 50%, 70%, 90%, or 95% of the backbone comprises phosphorothioate bonds. In some embodiments, 100% of the backbone comprises phosphorothioate bonds. In some embodiments, the oligonucleotide comprises a sequence lacking the capability of forming a hairpin structure at a temperature less than 25° C., 0° C., or less than −40° C. In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO:02. In some embodiments, the oligonucleotide comprises DNA. In some embodiments, the oligonucleotide comprises RNA. In some embodiments, the oligonucleotide is single-stranded.

In some embodiments, the nucleic acid sample is contacted with the carrier nucleic acids; wherein the nucleic acid sample comprises a target nucleic acid. In some embodiments, the carrier nucleic acids and the target nucleic acid are each derived from a genome of an organism of a kingdom, phylum, class, order, family, genus or species different from each other. In some embodiments, the carrier nucleic acids are derived from a genome of a fish, such as salmon. In some embodiments, the carrier nucleic acids comprise salmon sperm DNA, RNA, or siRNA. In some embodiments, the carrier nucleic acids have an average length less than 5000 consecutive nucleotides. In some embodiments, the carrier nucleic acids have an average length in a range from 100 to 5000 consecutive nucleotides. In some embodiments, the carrier nucleic acids have an average length in a range from 100 to 1000 consecutive nucleotides. In some embodiments, the carrier nucleic acids have an average length greater than 5000 consecutive nucleotides. In some embodiments, the carrier nucleic acids have an average length in a range from 5000 to 10000 consecutive nucleotides. In some embodiments, the target nucleic acid comprises an adaptor. In some embodiments, the adaptor comprises a nucleotide sequence selected from a P5 sequence (SEQ ID NO:05), a P7 sequence (SEQ ID NO:06), or a complement thereof. In some embodiments, the target nucleic acid has a concentration less than 10 nM, 100 pM, 20 pM, or 5 pM. In some embodiments, the target nucleic acid comprises (i) a bacteriophage nucleic acid; such PhiX, or (ii) a mammalian nucleic acid; such as a human nucleic acid. In some embodiments, the target nucleic acid comprises DNA. In some embodiments, the target nucleic acid is single-stranded.

### Systems and Kits

Some embodiments of the methods and compositions provided herein include kits and systems. Some embodiments include oligonucleotides and/or carrier nucleic acids provided herein. In some embodiments, a kit or system comprises an oligonucleotide or carrier DNA, and a nucleic acid sample. In some embodiments, the nucleic sample has concentrations less than concentration less than 500 nM, 100 nM, 10 nM, 100 pM, 20 pM, or 5 pM. Some embodiments include a substrate, such as a plurality of beads, such as magnetic beads, and the oligonucleotide. Some embodiments also include reagents and/or controls useful for sequencing nucleic acids, and/or preparing sequencing libraries. Examples of controls include those useful to normalize and/or calibrate sequencing systems.

In some embodiments, the oligonucleotide comprises 20, 40, 60 or more consecutive nucleotides. In some embodiments, the oligonucleotide comprises or consists of 60 consecutive nucleotides. In some embodiments, at least 50%, 70%, 90%, or 95% of the backbone comprises phosphorothioate bonds. In some embodiments, 100% of the backbone comprises phosphorothioate bonds. In some embodiments, the oligonucleotide comprises a sequence lacking the capability of forming a hairpin structure at a temperature less than 25° C., 0° C., or less than −40° C. In some embodiments, the oligonucleotide comprises a nucleotide sequence motif of [AAA(CT)_{X}]_{Y}, wherein X is 2 to 5, and Y is 2 to 6. In some embodiments, the oligonucleotide comprises at least 70%, 90%, 95%, or 100% sequence identity to the nucleotide sequence set forth in any one of SEQ ID NOs: 02-04. In some embodiments, the oligonucleotide comprises the nucleotide sequence set forth in SEQ ID NO:02. In some embodiments, the oligonucleotide comprises DNA. In some embodiments, the oligonucleotide comprises RNA. In some embodiments, the oligonucleotide is single-stranded. Some embodiments include blocked bead compositions provided herein.

In some embodiments, the carrier nucleic acids are derived from a genome of an organism of a kingdom, phylum, class, order, family, genus or species different from each other. In some embodiments, the carrier nucleic acids are derived from a genome of a fish, such as salmon. In some embodiments, the carrier nucleic acids comprise salmon sperm DNA, RNA, or siRNA. In some embodiments, the carrier nucleic acids have an average length less than 5000 consecutive nucleotides. In some embodiments, the carrier nucleic acids have an average length in a range from 100 to 5000 consecutive nucleotides. In some embodiments, the carrier nucleic acids have an average length in a range from 100 to 1000 consecutive nucleotides. In some embodiments, the carrier nucleic acids have an average length greater than 5000 consecutive nucleotides. In some embodiments, the carrier nucleic acids have an average length in a range from 5000 to 10000 consecutive nucleotides.

### EXAMPLES

### Example 1—Generation of Nucleic Acid Libraries

The following describes the preparation of human genome libraries for sequencing with (1) low-density bead-linked transposomes; (2) random mutagenesis; and (3) bottleneck (suppression) amplification. The workflow includes uniquely encoding long DNA templates with steps including highly uniform random mutagenesis and amplification. The long DNA templates are then fragmented and sequenced on a standard short-read platform. Reads from prepared libraries are used to accurately reconstruct and decode the original long template sequences using an unmutated reference data set which is generated in parallel.

Unmutated reference data: to reconstruct accurate long-read sequences from mutated short reads, an additional unmutated reference data set can be used. This can be generated from the same genomic starting material as the sample to be mutated, using standard methods for short-read library preparation and sequencing. Paired-end reads can be generated at a minimum length of 2×150 nucleotides for the unmutated data set, with a recommended 60× genome coverage for isolated bacterial genomes and 40× for pure human cell cultures.

Input DNA requirements: the workflow was found to be compatible with genomic DNA samples of relatively poor quality, containing unwanted low molecular weight fragments. These low molecular weight fragments are actively excluded by certain steps in the workflow; and the presence of some higher molecular weight material (>20 kb) is included to generate long templates for sequencing. To quantify input DNA for library preparation, a fluorometric-based method such as the Qubit dsDNA HS Assay Kit (Thermo Scientific) can be used. Concentrations of input DNA between 12.5 and 50 ng/μl can be used.

The following outlines a workflow which includes: high molecular weight tagmentation; mutagenesis PCR; library normalization; bottlenecking (suppression) PCR; library preparation; fragment analysis of products; and sequencing.

### Reagents, Materials and Thermocycler Parameters

| | |
|---|---|
| BLT Long Range | BLT-LR |
| Long Range PCR Mix | LPM |
| Long Range Marking Mix | LMM |
| Long Range Primer Mix 1 | LRP1 |
| Long Range Primer Mix 2 | LRP2 |
| Library Reducing Beads | LRB |
| Binding Buffer | IBB |
| Wash Buffer 1 | IWB1 |
| Stop Tagment Buffer 2 | ST2 |
| Illumina Purification Bead | IPB |
| PW1 (water) | |
| Resuspension Buffer | RSB |
| Ethyl Alcohol, Pure | |
| Enrich BL Transposome, Lg-LT | eBLT-L |
| Enhanced PCR Mix | EPM |
| Tagmentation Buffer 1 | TB1 |
| Tagmentation Wash Buffer | TWB |
| Nextera DNA UD Index Plate | |

Tagmentation parameters

| Step | Temperature | Time |
|---|---|---|
| 1 | 55° C. | 5 minutes |
| 2 | 10° C. | hold |

Mutagenesis PCR parameters

| Step | Temperature | Time |
|---|---|---|
| 1 | 68° C. | 3 minutes |

| | |
|---|---|
| 2 | PCR cycles (12 cycles) |

| | | |
|---|---|---|
| | 98° C. | 10 seconds |
| | 68° C. | 10 minutes |
| 3 | 4° C. | hold |

Bottlenecking PCR parameters

| | | |
|---|---|---|
| Step | Temperature | Time |

| | |
|---|---|
| 1 | PCR cycles (14 or 16 cycles) |

| | | |
|---|---|---|
| | 98° C. | 10 seconds |
| | 68° C. | 10 minutes |
| 3 | 4° C. | hold |

Index PCR parameters

| Step | Temperature | Time |
|---|---|---|
| 1 | 68° C. | 3 minutes |
| 2 | 98° C. | 3 minutes |

| | |
|---|---|
| 3 | PCR cycles (6 cycles) |

| | | |
|---|---|---|
| | 98° C. | 45 seconds |
| | 62° C. | 30 seconds |
| | 68° C. | 2 minute |
| 4 | 68° C. | 1 minutes |
| 5 | 4° C. | hold |

### High Molecular Weight Tagmentation

This step uses low-density bead-linked transposomes (BLT-LR) to generate long DNA fragments tagged with adapter sequences.

| | |
|---|---|
| 1 | Label a Biorad PCR plate. |
| 2 | Label a 1.7 ml tube and add the following components in order: BLT-LR; and |
| | TB1. |
| 3 | Vortex to mix thoroughly, then briefly pulse centrifuge. |
| 4 | Add 20 μl of mastermix to wells. |
| 5 | Add 30 μl Input DNA (50 ng total). |
| 6 | Use a pipette to gently mix then briefly pulse centrifuge. |
| 7 | Place tubes in a thermocycler with a heated lid set to 80° C. and run the Tagment. |
| 8 | Remove from the thermocycler and add 10 μl of ST2. |
| 9 | Use a pipette set at half of the total rxn volume to gently mix. |
| 10 | Incubate at room temperature for 2 min. |
| 11 | Place the tubes on a magnetic stand or plate for 3 min or until the solution is |
| | clear. Remove and discard the clear supernatant. Note: If beads become |
| | disturbed during aspiration, redisperse solution into the tube. Maintain tube on |
| | the magnet to let the beads settle. |
| 12 | Remove tubes from the magnet and add 100 μl TWB. Gently pipette mix until |
| | beads are fully resuspended. |
| 13 | Place the tubes on a magnetic stand or plate and incubate for at least 2 min, or |
| | until clear. While samples are incubating, proceed to the next step. Note: Keep |
| | pellets in TWB to help prevent any chance of over-drying the beads. |

### Mutagenesis PCR

In this step, long templates are uniquely encoded via random incorporation of the mutagenic nucleotide analogue dPTP during PCR.

| | |
|---|---|
| 14 | Label a 1.7 ml tube and combine the following to prepare mutagenesis master |
| | mix: LMM; LPM; LRP1; and RSB. |
| 15 | Use a pipette to mix thoroughly, then briefly pulse centrifuge. |
| 16 | Remove TWB from each well. |
| 17 | Add 50 μl of master mix to beads. |
| 18 | Gently pipette mix to resuspend beads |
| 19 | Place in preprogrammed thermal cycler and run the Mutagenesis PCR program. |
| 20 | Remove from thermocycler. |
| 21 | Vortex IPB to resuspend the beads. |
| 22 | Add 30 ul of IPB beads to each well. |
| 23 | Mix the total reaction volume by gently pipetting at least 10 times and incubate |
| | for 2 minutes at room temperature. |
| 24 | Place on the magnetic stand and wait until the liquid is clear (~5 minutes). |
| 25 | Remove and discard all supernatant, |
| 26 | Wash beads as follows: a. keep on the magnetic stand and add 180 μl fresh 80% |
| | EtOH to each well; b. wait 30 seconds; and c. remove and discard all |
| | supernatant |
| 27 | Briefly spin for 10 seconds and return plate to magnet. |
| 28 | With a 20 μl pipette, remove all residual EtOH. |
| 29 | Dry on magnet for 2 minutes. |
| 30 | Remove from the magnetic stand and add 25 μl RSB to each well. |
| 31 | Pipette mix and incubate at room temperature for 2 minutes. |
| 32 | Place on the magnetic stand and wait until the liquid is clear (~2 minutes). |
| 33 | Transfer 10 μl supernatant to a new plate and leave remaining supernatant with |
| | SPRI beads |

### Library Normalization

| | |
|---|---|
| 34 | In a new 2 ml tube, combine the following to prepare normalization master mix: |
| | Binding Buffer (IBB); and LRB |
| 35 | Vortex normalization master mix until homogenous immediately prior to use. |
| 36 | Pipette 10 uL of prepared mastermix into each well containing 10 uL of DNA. |
| 37 | Seal plate with microseal and put on bioshake for 30 minutes at 1800 rpm. |
| 38 | Remove plate from bioshake and place on plate magnet for 1 minute. Removing |
| | and replacing plate on magnet may help to condense beads. |
| 39 | Gently remove supernatant, taking care not to disturb beads. |
| 40 | Remove plate from magnet. |
| 41 | Immediately add 150 uL of IWB1 to each well. |
| 42 | Using a p200 pipette set to 140 uL, pipette mix each well at least 10 times. |
| 43 | Place on plate magnet for 1 minute. |
| 44 | Gently remove supernatant, taking care not to disturb beads. |
| 45 | Repeat steps 41-44 for a total of two washes. Remove plate from magnet. |
| 46 | Immediately add 150 uL of 80% EtOH to each well. |
| 47 | Using a p200 pipette set to 140 uL, pipette mix each well at least 10 times. |
| 48 | Place on plate magnet for 1 minute. |
| 49 | Gently remove supernatant, taking care not to disturb beads. |
| 50 | Briefly spin down plate and use a p20 to remove excess liquid. |
| 51 | Allow to dry for 2 minutes. |
| 52 | Add 80 uL of RSB to each sample well. |
| 53 | Using a pipette set to 50 uL, pipette mix each sample at least 10 times. |
| 54 | Allow to incubate for 10 minutes. |
| 55 | Place on plate magnet for 1 minute. |
| 56 | Transfer 70 uL of supernatant to new well. |
| 57 | If performing 165 pg input, go to step 62 for next |
| | If performing 55 pg input, dilute 20 ul of eluted material in 40 ul of RSB in |
| | adjacent well. Mix well, then use 2 ul as stated in step 62. |
| | [Optional] Measure DNA on Qubit using 10 uL of DNA diluted in 190 uL of |
| | Qubit buffer. |

### Bottleneck (Suppression) PCR

In this step a defined quantity of the purified mutagenesis product is amplified to create many copies of each unique template. The amount of starting material in the bottleneck PCR determines the number of long templates available for sequencing, and is controlled through careful dilution of the mutagenesis sample. The following protocol can be used to generate between about 10× to about 30× long-read coverage of the human genome (see below). For arbitrary samples, a simple calculator or look up table could be provided to guide users on sample dilution and indicate the number of enrichment cycles required for a particular genome size or sample type.

| | |
|---|---|
| 58 | Combine the following volumes to prepare enrichment master mix: LPM; and |
| | LRP2. |
| 59 | Use a pipette to mix thoroughly, then briefly pulse centrifuge. |
| 60 | Add 48 μl of master mix to a new Bio-Rad plate. Place tubes on ice. |
| 61 | Add 2 μl of DNA to each well. |
| 62 | Gently pipette mix the 50 μl reaction and briefly pulse centrifuge. |
| 63 | Place in preprogrammed thermal cycler and run the Bottlenecking PCR program. |
| 64 | Note: Perform 16 enrichment cycles for 10× genome coverage or 14 cycles for |
| | 30×. |
| 65 | Remove from thermocycler. |
| 66 | Vortex IPB to resuspend the beads. |
| 67 | Add 30 ul of IPB beads to each well. |
| 68 | Mix the total reaction volume by gently pipetting at least 10 times and incubate |
| | for 2 minutes at room temperature. |
| 69 | Place on the magnetic stand and wait until the liquid is clear (~5 minutes). |
| 70 | Remove and discard all supernatant. |
| 71 | Wash beads as follows: a. keep on the magnetic stand and add 180 μl fresh 80% |
| | EtOH to each well; b. wait 30 seconds; and c. remove and discard all supernatant. |
| 72 | Briefly spin for 10 seconds and return plate to magnet. |
| 73 | With a 20 μl pipette, remove all residual EtOH. |
| 74 | Dry on magnet for 2 minutes. |
| 75 | Remove from the magnetic stand and add 25 μl RSB to each well. |
| 76 | Pipette mix and Incubate at room temperature for 2 minutes. |
| 77 | Place on the magnetic stand and wait until the liquid is clear (~2 minutes). |
| 78 | Transfer 24 μl supernatant to a new plate. |
| 79 | Keep on ice for same day use or store at −20° C. (safe stopping point). |
| 80 | Determine the concentration of each purified bottlenecking PCR product with the |
| | Qubit dsDNA HS Assay Kit. Use 1-2 μl of sample DNA per measurement. |

### Library Preparation

In this step the long, mutated DNA templates are fragmented and adapters are attached to create a library of short, overlapping fragments that are ready for sequencing. 50 ng of each purified enrichment product can be used as input DNA for internal library preparation. A small amount of bottlenecking PCR product can be reserved for subsequent analysis of template size.

| | |
|---|---|
| 81 | Bring TB1 and eBLT-L to room temperature and vortex to mix. |
| 82 | Make a dilution using RSB and 50 ng worth of purified bottlenecking product |
| | into each well with a final volume of 30 μl. |
| 83 | Vortex eBLT-L vigorously for 10 seconds, then visually check the beads for |
| | complete resuspension. Repeat as necessary. |
| 84 | Prepare a tagmentation master mix: eBLT-L; TB1. |
| 85 | Vortex the tagmentation master mix thoroughly to make sure the eBLT-L |
| | beads are evenly resuspended in the buffer. |
| 86 | Using fresh tips, transfer 20 μl of tagmentation master mix to each tube |
| | containing 30 μl of DNA. |
| 87 | Pipette mix the 50 μl reaction mix to resuspend. |
| 88 | Place in preprogrammed thermal cycler and run the Tagment program. |
| 89 | Remove from thermocycler and add 10 μl of ST2 to each tagmentation |
| | reaction. |
| 90 | Use a pipette set at half of the total rxn volume to gently mix. |
| 91 | Incubate at RT for 5 minutes. |
| 92 | Place the tubes on a magnetic stand or plate for 3 min or until the solution is |
| | clear. Remove and discard the clear supernatant. Note: If beads become |
| | disturbed during aspiration, redisperse solution into the tube. Maintain tube on |
| | the magnet to let the beads settle. |
| 93 | Remove tubes from the magnet and add 100 μl TWB. Gently pipette mix until |
| | beads are fully resuspended. |
| 94 | Place the tubes on a magnetic stand or plate for 3 min or until the solution is |
| | clear. Remove and discard the clear supernatant. |
| 95 | Remove tubes from the magnet and add 100 μl TWB. Gently pipette mix until |
| | beads are fully resuspended (total of 2 washes). |
| 96 | Place the tubes on a magnetic stand or plate and incubate for at least 3 min, or |
| | until clear. While samples are incubating, proceed to the next step. Note: Keep |
| | pellets in TWB to help prevent any chance of over drying the beads. |
| 97 | Prepare a index PCR master mix: EPM; RSB. |
| 98 | Use a pipette to mix thoroughly, then briefly pulse centrifuge. |
| 99 | Carefully remove the second TWB wash from the samples while on the |
| | magnet. Use a pipette with P10 or P20 tips to remove excess liquid from the |
| | tube. Any remaining foam on the tube walls does not adversely affect the |
| | library |
| 100 | Remove tubes from the magnet and proceed immediately to the next step to |
| | prevent excessive drying of the beads. |
| 101 | Add 46 μl of the PCR master mix to each sample. Gently pipette mix to ensure |
| | the beads are thoroughly resuspended. |
| 102 | To each sample, add 4 μl Nextera DNA UD Index. Use a unique combination |
| | of indexes for each sample. |
| 103 | Using a pipette set to 40 μl, pipette mix a minimum of 10 times to mix the |
| | entire reaction volume. |
| 104 | Place in preprogrammed thermal cycler and run the Index PCR program. |
| 105 | Clean up PCR reaction using IPB beads as follows: |
| 106 | Place plate on the magnetic stand and wait until the liquid is clear (~5 |
| | minutes) |
| 107 | Transfer 45 μL of supernatant to a new plate. |
| 108 | Add 55 uL RSB to bring volume to 100 uL. |
| 109 | Perform a double-sided bead purification as follows: |
| 110 | Add 30 uL of IPB beads to the sample. |
| 111 | Mix the total reaction volume by gently pipetting at least 10 times and |
| | incubate for 2 minutes at room temperature. |
| 112 | Place on the magnetic stand and wait until the liquid is clear (~5 minutes). |
| 113 | Transfer 127 μl of the clear superatant in new wells. Discard the remaining |
| | beads. |
| 114 | Add 20 ul of IPB beads to the sample. |
| 115 | Mix the total reaction volume by gently pipetting at least 10 times and |
| | incubate for 2 minutes at room temperature. |
| 116 | Place on the magnetic stand and wait until the liquid is clear (~5 minutes). |
| 117 | Remove and discard all supernatant. |
| 118 | Wash beads as follows: a. keep on the magnetic stand and add 180 μl fresh |
| | 80% EtOH to each well; b. wait 30 seconds; and c. remove and discard all |
| | supernatant. |
| 119 | Briefly spin for 10 seconds and return plate to magnet. |
| 120 | With a 20 μl pipette, remove all residual EtOH. |
| 121 | Dry on magnet for 2 minutes. |
| 122 | Remove from the magnetic stand and add 30 μl RSB to each well. |
| 123 | Pipette mix and Incubate at room temperature for 2 minutes. |
| 124 | Place on the magnetic stand and wait until the liquid is clear (~2 minutes). |
| 125 | Transfer 28 μl to a new plate. |
| 126 | Determine the concentration of each purified enrichment product with the |
| | Qubit dsDNA HS Assay Kit. Use 1-2 μl of sample DNA per measurement. |
| 127 | Keep on ice for same day use or store at −20° c. (SAFE STOPPING POINT). |

Libraries can now be quantified, normalized and sequenced using standard workflows for libraries. Some additional considerations for quality control, sample index selection and sequencing are provided below.

### Fragment Analysis of Products

| | |
|---|---|
| 128 | Run purified library prep product on appropriate instrument for |
| | fragment analysis. |
| 129 | Record the average fragment length for the purified library prep |
| | product. |

DNA fragment length: assessing the fragment length profile of the purified bottlenecking PCR product can be performed to evaluate the size distribution of long templates as well as to evaluate the final short-read library. To assess the fragment size of the purified bottlenecking PCR product, the following products from Agilent Technologies® can be used: Bioanalyzer 2100, TapeStation 4200, Fragment Analyzer 5300, or equivalent technologies from other providers.

Purified Bottlenecking Product

| | | | Expected average |
|---|---|---|---|
| | | Recommended | fragment length |
| Instrumentation | Kit | Region (bp) | (bp) |
| Bioanalyzer | High Sensitivity | 3,000-10,000 | 7,000-8,000 |
| 2100 | DNA | | |
| Fragment | DNF-464 HS Large | 3,000-49,000 | 7,000-8,000 |
| Analyzer 5300 | Fragment | | |

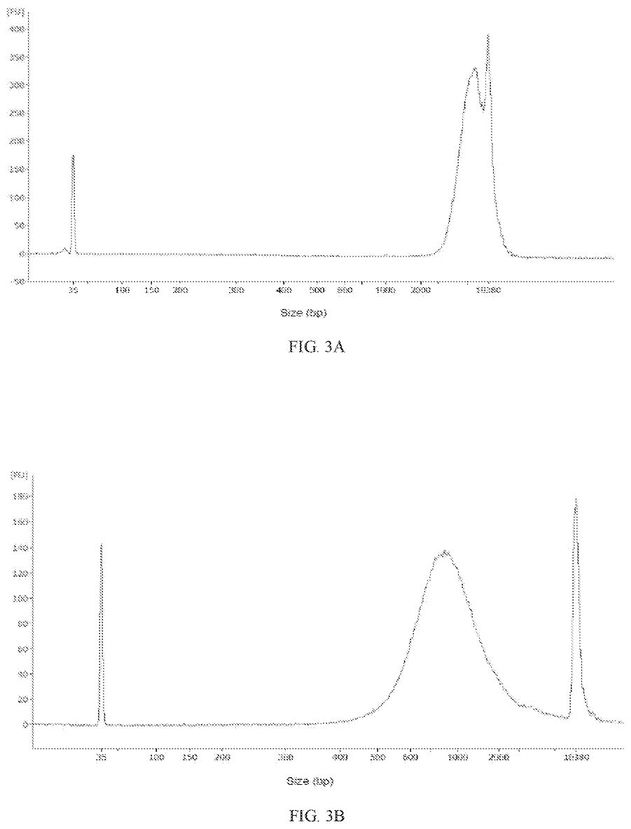

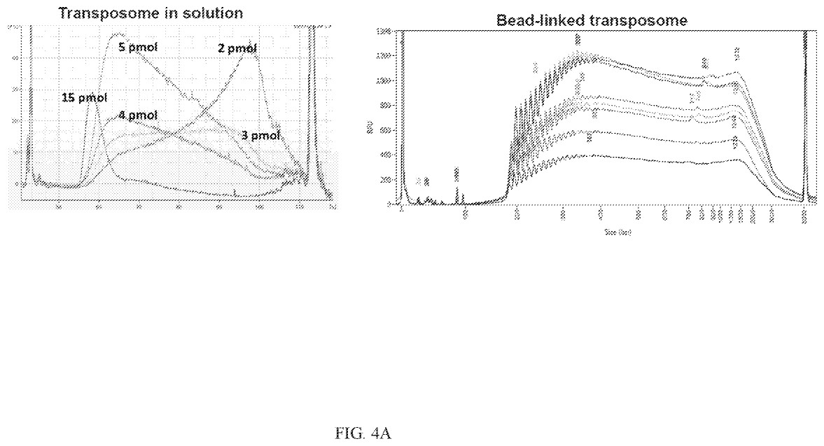

The peak template length after bottlenecking PCR is expected to be around 7,000-8,000 bp, with virtually no products below ˜3,000 bp. FIG. 3A illustrates purified bottlenecking PCR product run on an Agilent® Bioanalyzer using a High Sensitivity DNA Kit.

Final Library

| | | Region | Expected average |
|---|---|---|---|
| Instrumentation | Kit | (bp) | fragment length (bp) |
| TapeStation | High Sensitivity | 150-1,500 | 800-900 |
| 4200 | D5000 | | |
| Bioanalyzer | High Sensitivity DNA | 150-1,500 | 800-900 |
| 2100 | | | |

The peak template length after bottlenecking PCR is expected to be around 800-900 bp. FIG. 3B illustrates a purified final library preparation product run on an Agilent® Bioanalyzer using a High Sensitivity DNA Kit.

### Sequencing

Sequence the final library or library pool on a NGS instrument, generating 2×150 nt paired end reads. Aim to produce at least 400 Gbp of sequence data for mutated samples targeting 10× long-read coverage of the human genome, or at least 1200 Gbp for 30× coverage. This is in addition to the unmutated reference data that is also required for long read reconstruction.

### Example 2—Effects of Immobilizing Transposomes on Beads at Low Densities

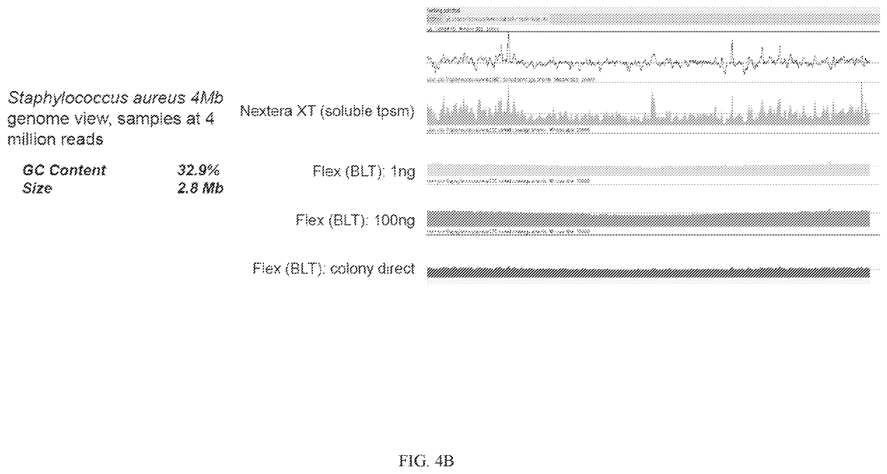

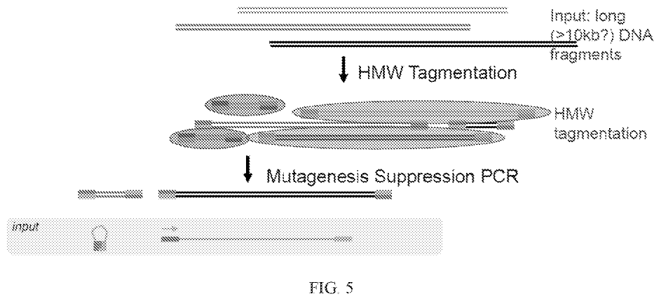

This example illustrates improved long-read coverage by changing initial tagmentation from soluble transposomes to low-density bead-linked transposomes (BLT-LR); and changing from an A14/B15 mixture of BLT-LRs to B15 BLT-LR only. Nucleic acid libraries were generated and sequenced with a protocol substantially similar to that of Example 1. Different amounts of input DNA were tested. A protocol using bead-linked transposomes was compared with a protocol using transposomes in solution. As shown in FIG. 4A and FIG. 4B, a switch from low concentration soluble transposomes to low-density BLT (BLT-LR) provided increased robustness to changes in transposome: input DNA ratio; and a more uniform coverage with BLT vs. soluble.

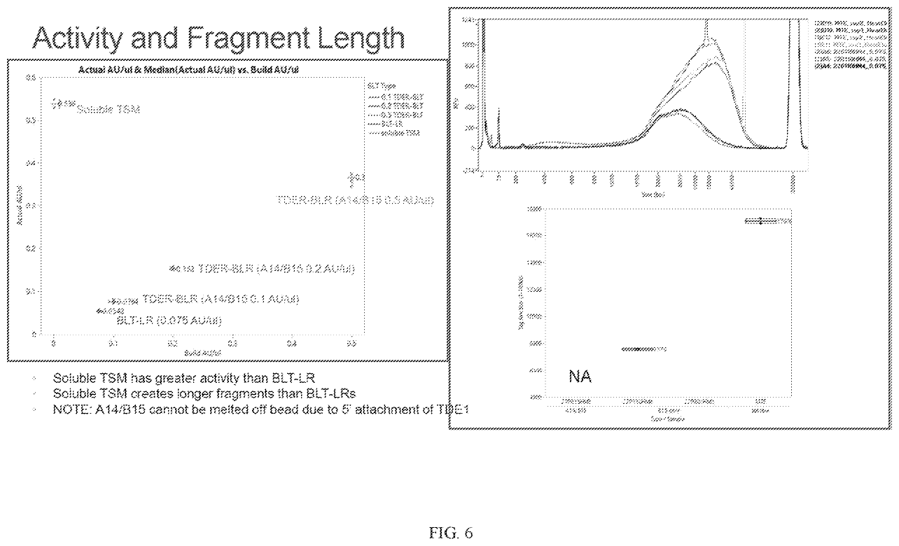

FIG. 5 outlines steps of high molecular weight tagmentation followed by mutagenesis and suppression PCR to enrich for longer fragments.

Protocols were compared that included (i) soluble transposome (TSM) (0.4 AU/ul); (2) BLT-LR made with A14/B15 (0.1 AU/ul build); or (3) BLT-LR made with B15 only (0.075 AU/ul build). Quality control and sequencing metrics were compared for each protocol.

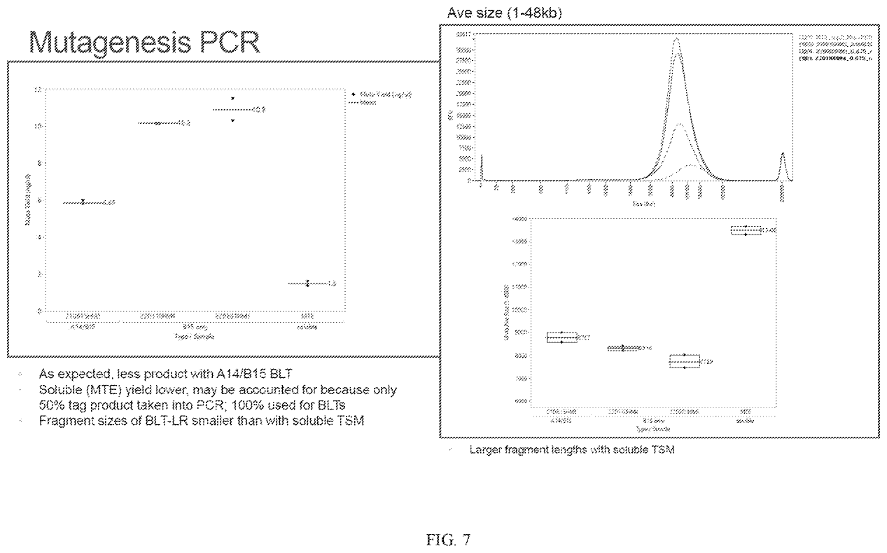

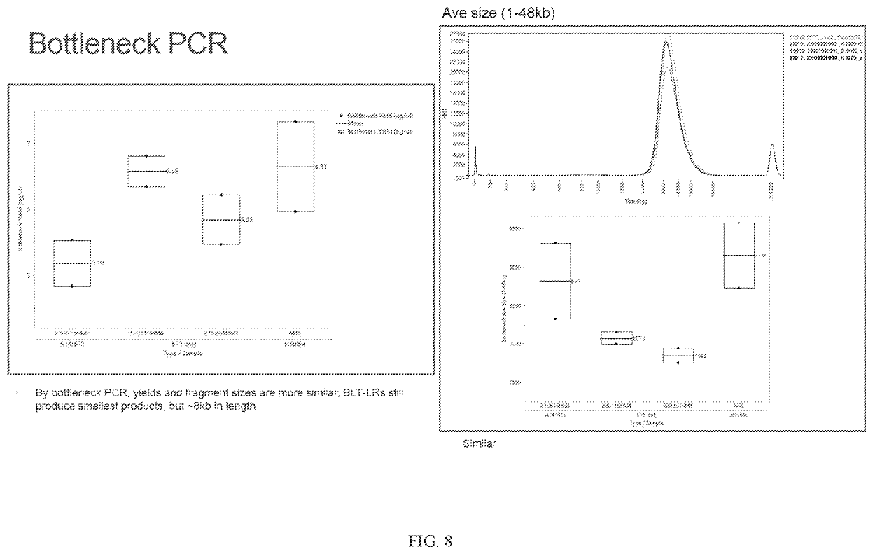

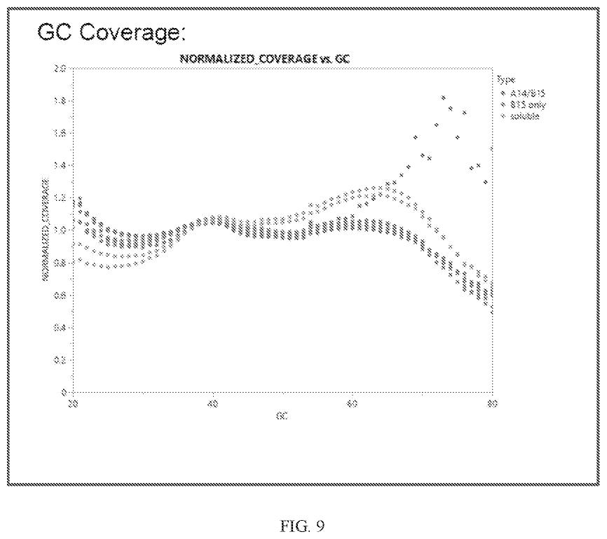

As shown in FIG. 6, soluble TSM had a greater activity than BLT-LR and soluble TSM created longer fragments than BLT-LRs. A14/B15 could not be melted off bead due to 5′ attachment of TDE1. In mutagenesis PCR, A14/B15 BLT provided a lower yield than B15 only (FIG. 7). The yield with soluble (MTE) yield was also lower and may have been accounted for because only 50% tag product was taken into PCR; 100% used for BLTs. Fragment sizes of BLT-LR smaller than with soluble TSM. In bottleneck (suppression) PCR, yields and fragment sizes were more similar, while BLT-LRs still produced smallest products of ˜8 kb in length (FIG. 8).

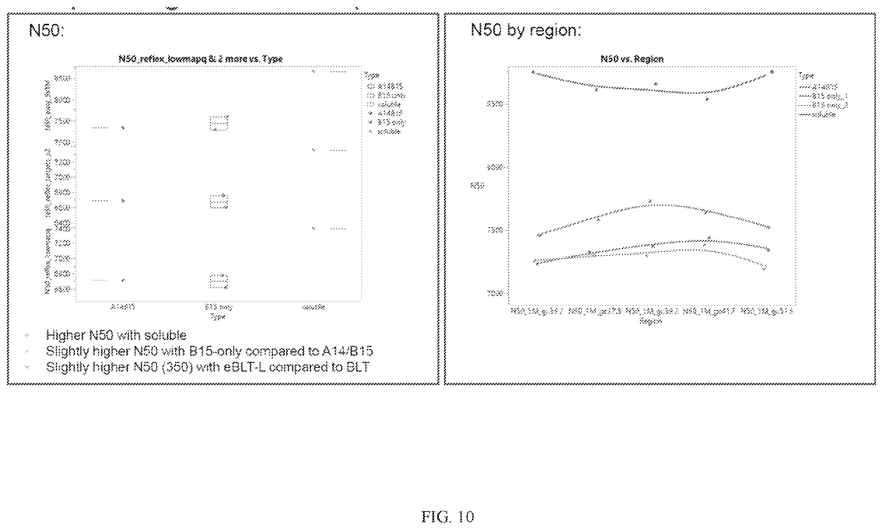

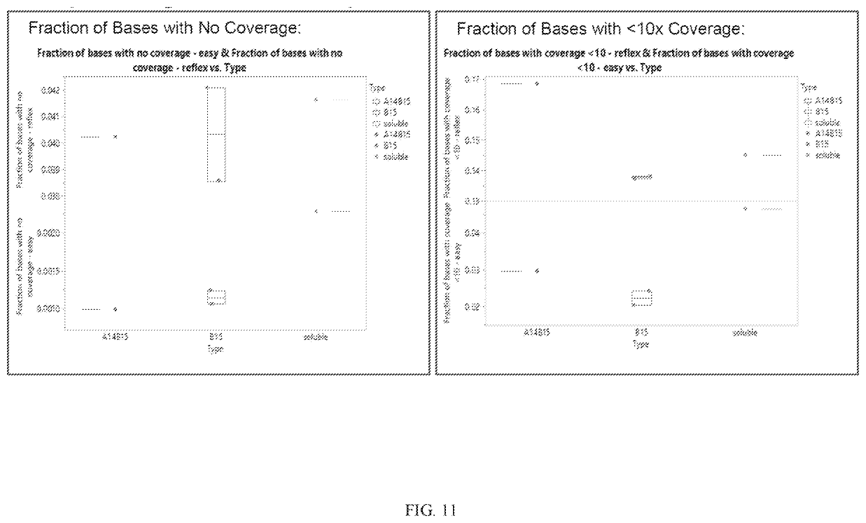

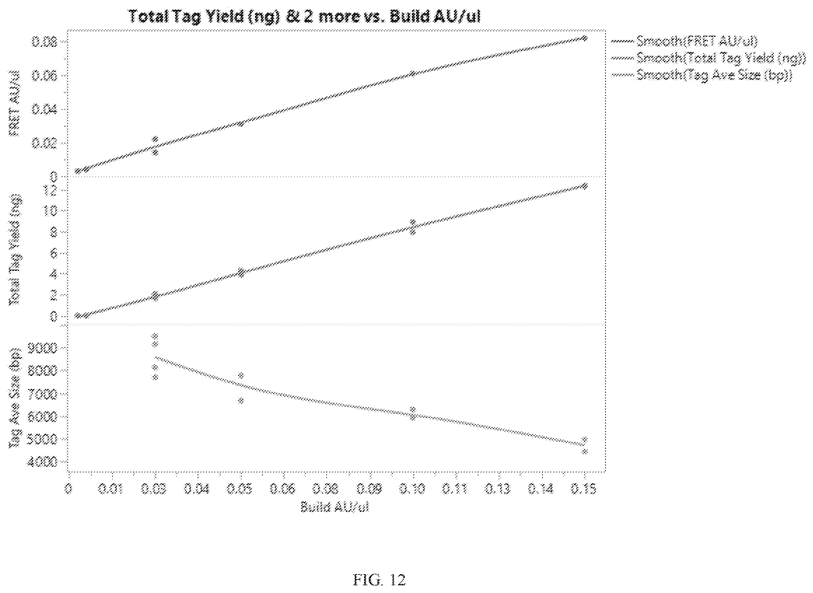

In a sequencing metrics comparison, GC bias was comparable between soluble and BLT-LR with A14/B15 BLT slightly worse (FIG. 9) A higher N50 was achieved with soluble TSM, slightly higher N50 with B15-only compared to A14/B15, slightly higher N50 (350) with eBLT-L compared to BLT (FIG. 10). Sequencing redundancy, SLR depth, and error rate achieved using with A14/B15 BLT or B15 only BLT were measured. Fraction of bases with no coverage achieved using with A14/B15 BLT or B15 only BLT is depicted in FIG. 11.

BLT-LRs had better coverage than soluble transposomes (fraction of bases with <0/10× coverage). BLT-LRs created shorter fragments, which generated lower N50s in workflow. N50s were still above 5 kb mark so this decrease in performance was acceptable when paired with better coverage metrics and a more robust tagmentation reaction. B15-only TSM BLTs gave a better yield and lower redundancy than A14/B15 BLTs. The change to B15 BLT-LRs created a more efficient mutagenesis suppression PCR.

### Example 3—Effects of BLT Activity on Fragment Size

BLT-LR activity was investigated for high molecular weight tagmentation. BLT-LR activity should provide: tagment large fragments to provide for mutagenesis PCR; maximize fragment size, ideally >8 kb; yield >4 ng post-high molecular weight (HMW) tagmentation; reproducibility; ease in QC tested; and good sequence quality. A goal was to maximize fragment size while maintaining good yield and downstream sequencing metrics

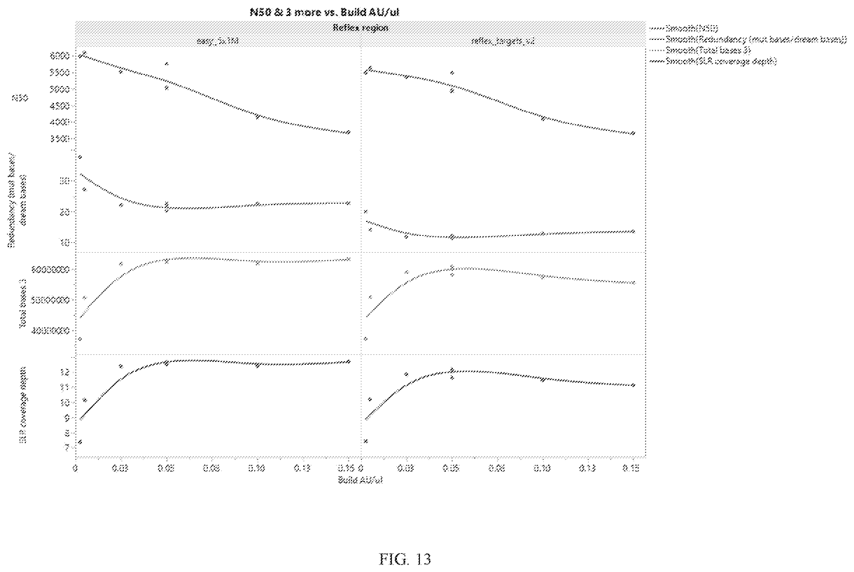

BLT-LRs having different levels of activity were compared. As transposome activity (AU/ul) decreased, yield decreased and average fragment size increased (FIG. 12).

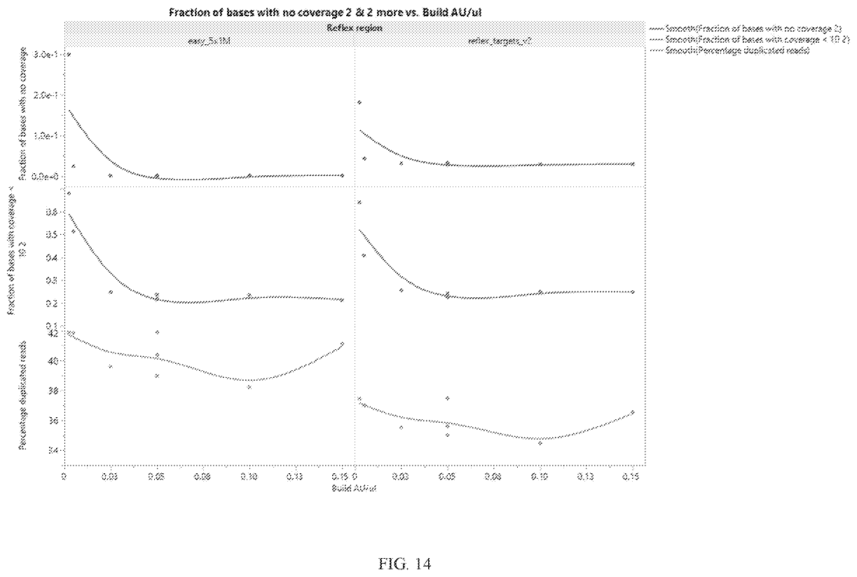

BLT-LRs were compared with builds/activities of 0.0025, 0.005, 0.025, 0.05, 0.1, 0.15 AU/ul. N50s were compared. “N50” was the length of the shortest contig for which longer and equal length contigs cover at least 50% of the assembly. Lower build activity maximized N50s but sequencing metrics started to drop at 0.025 AU/ul (FIG. 13).

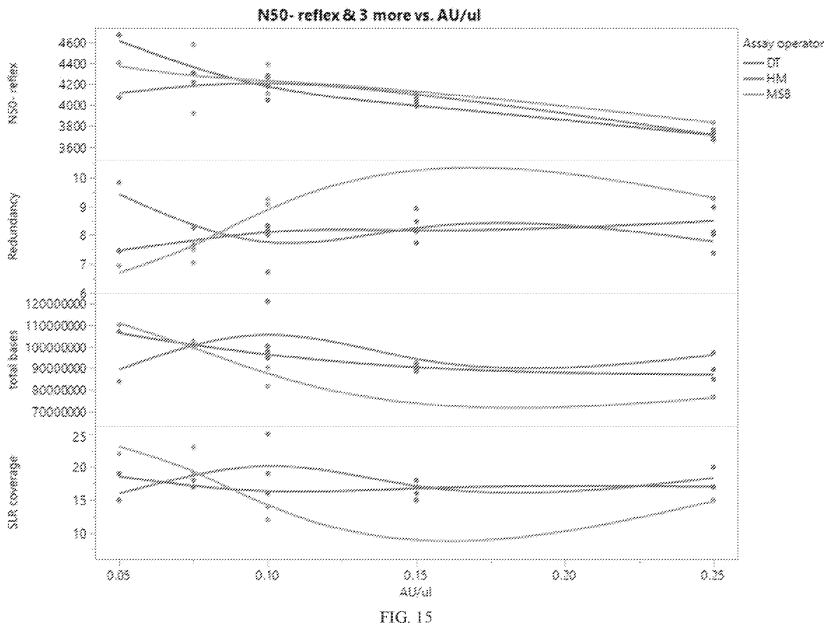

Lower build activity maximized N50s but sequencing metrics started to decrease at 0.025 AU/ul (FIG. 14). There was no apparent cliff-edge on high activity side, but N50s continued to decline, and at 0.075 AU/ul, well above cliff edge while maximizing N50.

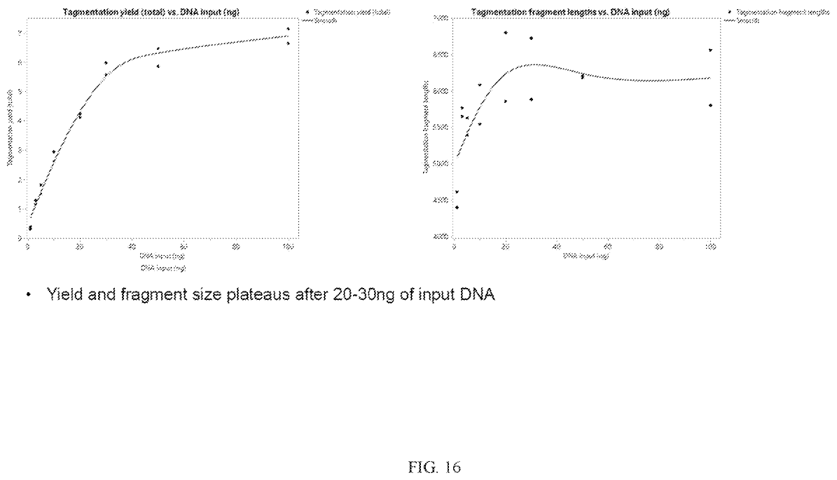

Results were compared from studies with three different operators testing activities from 0.05 AU/ul-0.25 AU/ul. Consistent performance between operators was found for BLT-LR activities from 0.05-0.25 AU/ul (FIG. 15). A BLT-LR activity of 0.075 AU/ul was chosen for BLT-LR which balanced fragment size and yield. It was found that a fluctuation of +/−100% in activity would still provide good sequencing metrics.

### Example 4—Effects of Quantity of Input DNA

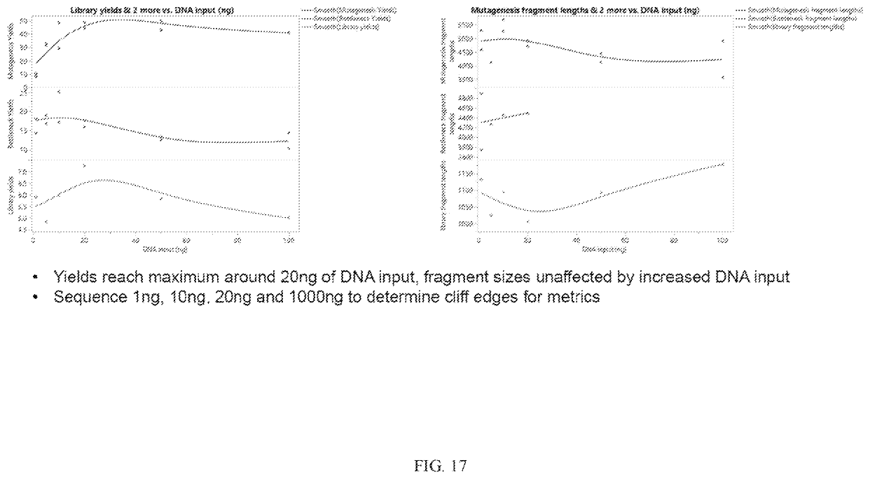

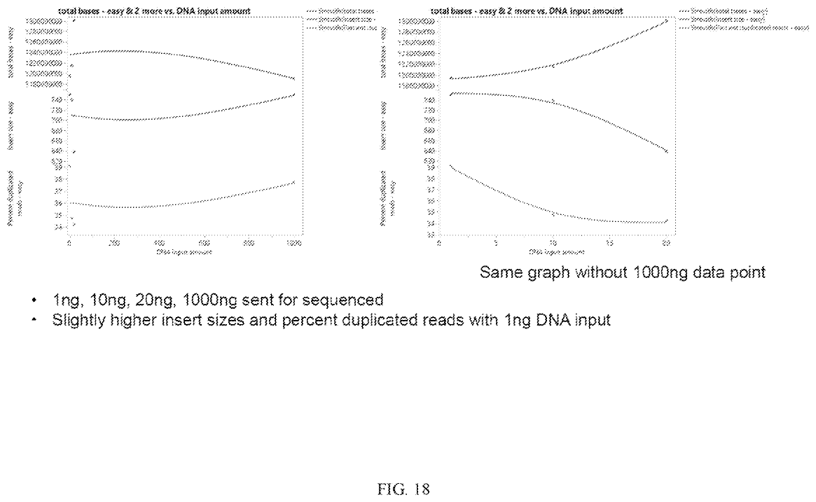

Changing the amount of input DNA used in the initial HMW tagmentation reaction could impact any of the following: amount of DNA tagmented (test BLT-LR saturation); fragment sizes after initial HMW tagmentation (and downstream); biases in what is tagmented/amplified; sequencing metrics including percent duplicates, redundancy, N50, GC bias. Effects of input DNA quantity were tested for a protocol substantially similar to the workflow of Example 1 for amounts: 1 ng, 3, ng, 5 ng, 10 ng, 20 ng, 30 ng, 50 ng, 100 ng, 300 ng, and 1000 ng. Yield and fragment size plateaued after 20-30 ng of input DNA (FIG. 16). Yields reached maximum around 20 ng of DNA input, fragment sizes were unaffected by increased DNA input (FIG. 17).

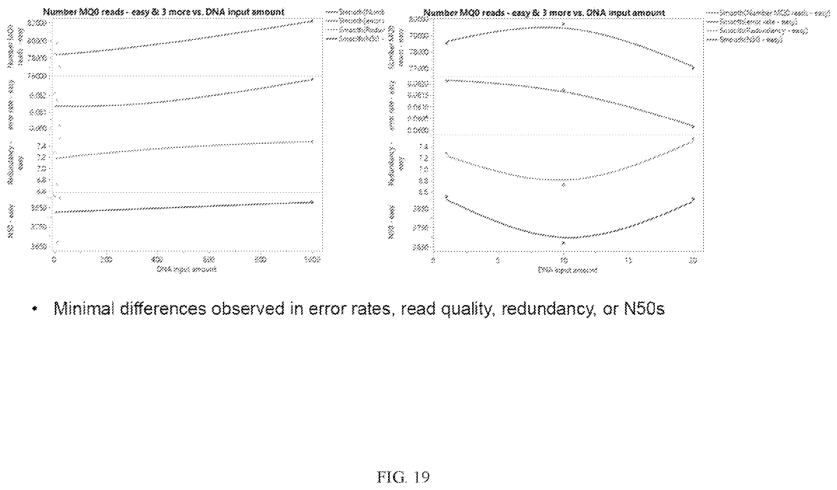

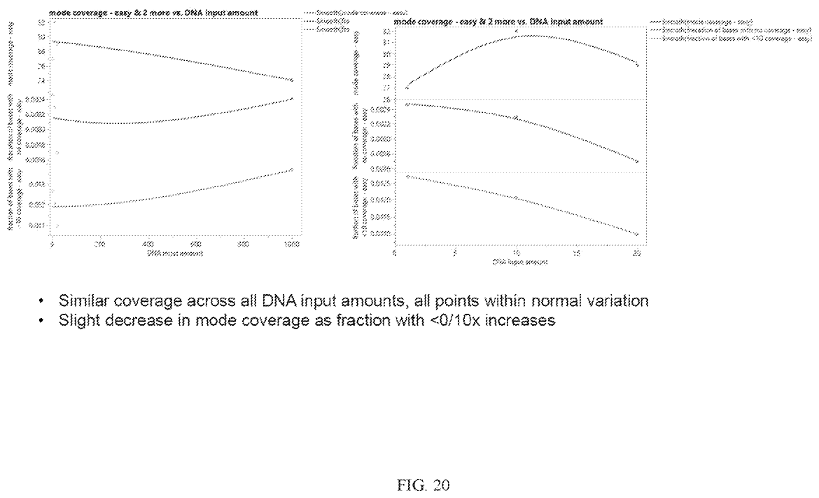

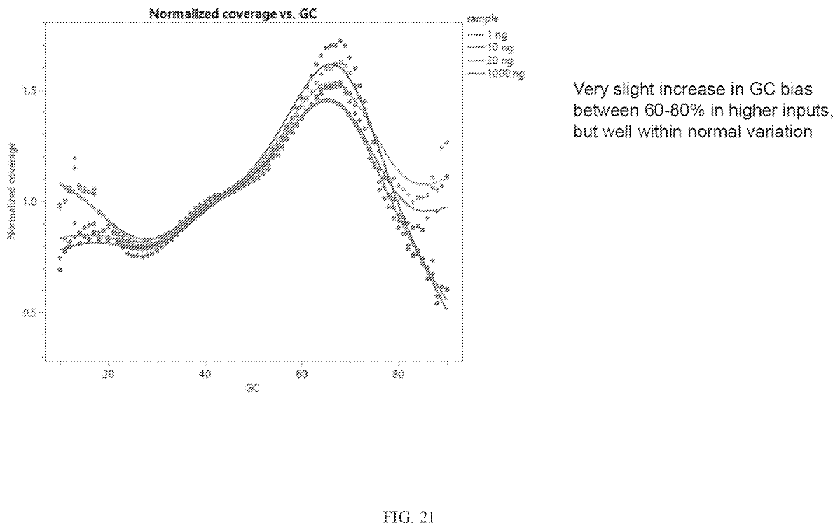

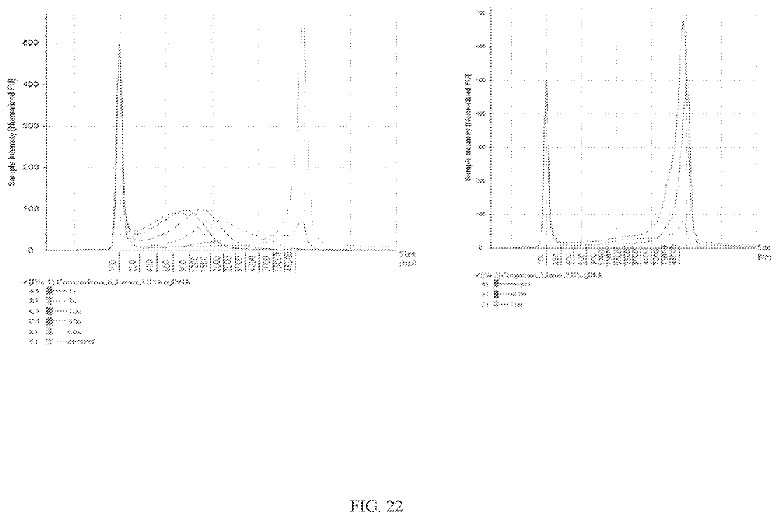

Sequencing was performed for input DNAs of 1 ng, 10 ng, 20 ng and 1000 ng to determine cliff edges for metrics. A workflow with 1 ng input DNA provided slightly higher insert sizes and percent duplicated reads compared to workflows performed with higher amounts of input DNA (FIG. 18). Minimal differences were observed in error rates, read quality, redundancy, or N50s (FIG. 19). Similar coverage across all DNA input amounts were observed with all points within normal variation with a slight decrease in mode coverage as fraction with <0/10× increased (FIG. 20). A very slight increase in GC bias between 60-80% was observed in higher inputs, but well within normal variation (FIG. 21).

In sum, similar sequencing metrics were observed for input DNA amounts of 1 ng to 1000 ng. Inputs as low as 1 ng were feasible for the workflow. BLT-LR saturated at about 30 ng input; input above 30 ng did not bias coverage. DNA input amounts less than 10ng resulted in low yields in the workflow. There was a slight increase in percent duplicated reads for 1 ng DNA input.

### Example 5—Effects of Quality of Input DNA

Effects of input DNA quality was tested for a protocol substantially similar to the workflow of Example 1. Preliminary data indicated that vortexing and up to 3 freeze/thaw cycles for input DNA were tolerated; and FFPE DNA was not suitable/too degraded as input DNA. Input DNA was prepared by shearing for 1, 3, 10, 30 and 60 seconds, and compared to control BLT-LR tagmented DNA and HMW DNA.

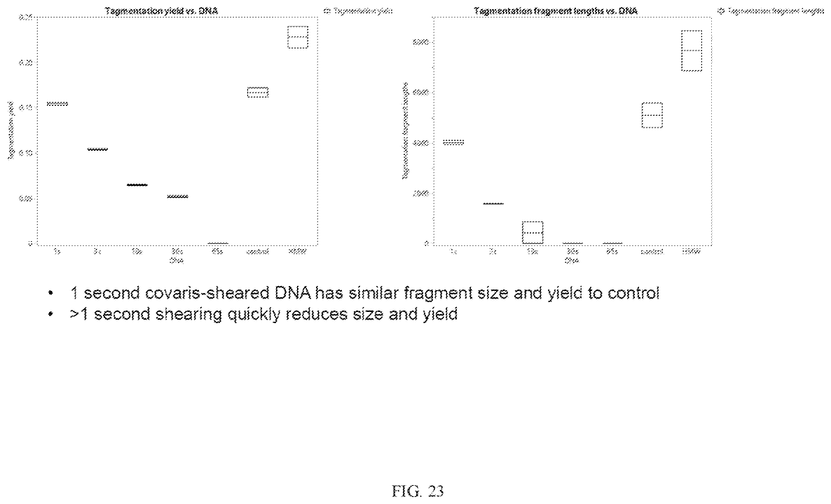

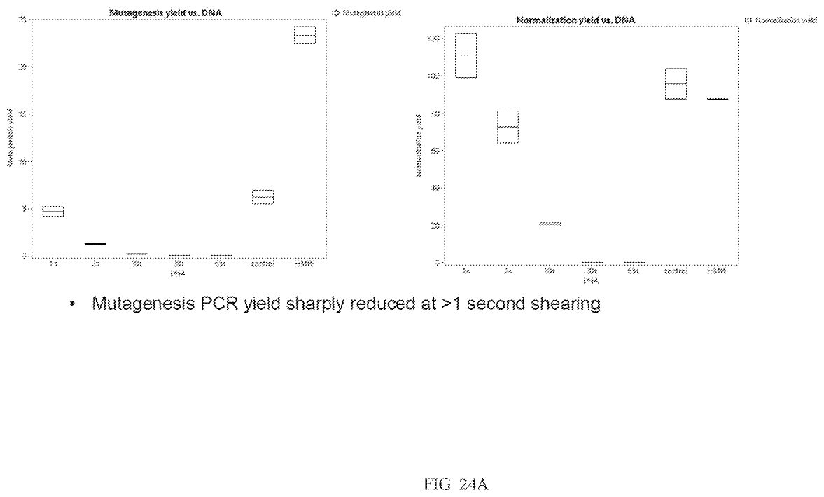

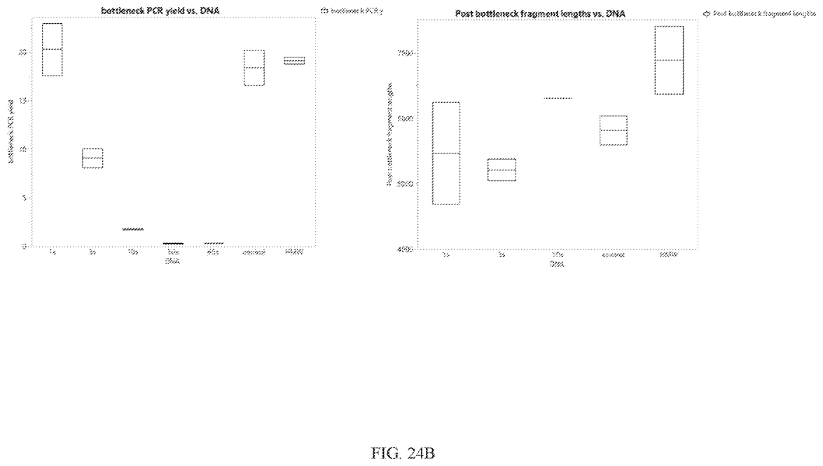

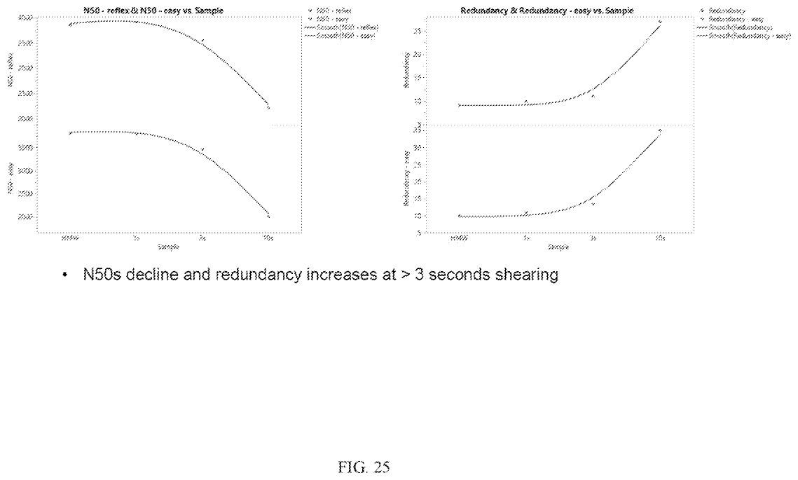

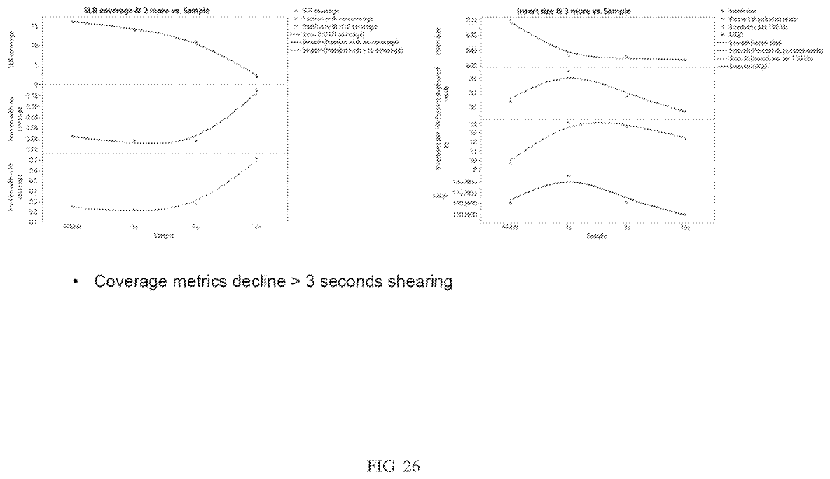

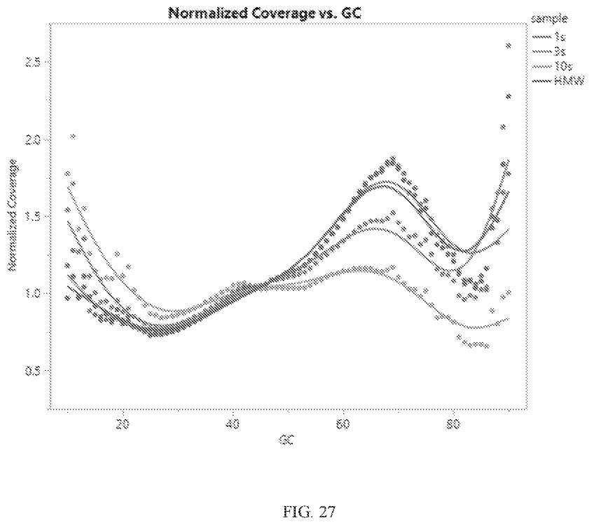

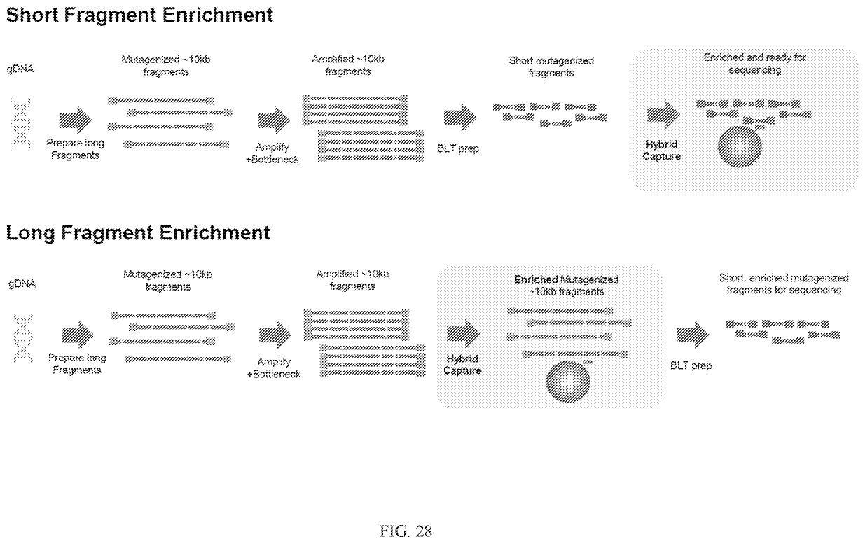

Input DNA was sheared for 1, 3, 10, 30 and 60 seconds. There was a noticeable change in size distribution profile after even 1 second, while Control BLT-LR and HMW DNA gave similar size profiles (FIG. 22). In the tagmentation step of the workflow, 1 second sheared DNA had similar fragment size and yield to control, and >1 second shearing quickly reduced size and yield (FIG. 23). In the mutagenesis step of the workflow, mutagenesis PCR yield sharply reduced at >1 second shearing (FIG. 24A). In the bottleneck PCR step of the workflow, yield also sharply reduced at >1 second shearing (FIG. 24B). For sequencing metrics, N50s declined and redundancy increased at >3 seconds shearing (FIG. 25). Coverage metrics declined >3 seconds shearing (FIG. 26). GC bias correlated with post-tagmentation sizes (FIG. 27).

In sum, HMW DNA gave better yields and larger fragment lengths coming out of initial Tagmentation but did not result in final higher N50. Highly sheared DNA (30 s or longer) did not amplify well in either PCR step which resulted in not enough DNA to continue with library prep. N50s and coverage/redundancy metrics worsened with DNA sheared >3 seconds. GC bias was impacted by fragment sizes. PCR steps were not suitable for highly degraded DNA, but tolerated mild shearing (1-3 sec) reasonably well

BLT-LRs were investigated to create large fragment sizes suitable for the workflow outlined in Example 1. B15-only transposomes improved mutagenesis PCR small-fragment suppression and overall yield The workflow gave improved coverage of low MapQ regions of the genome. The workflow was robust to changes in DNA input amount. The workflow tolerated mildly sheared DNA, DNA that has been through freeze/thaw, and DNA that had been vortexed.

### Example 6—Enriching for Long Fragments

A workflow was performed, as described in Example 1, with an added enrichment step. The workflow included: high molecular weight tagmentation; mutagenesis PCR; library normalization; bottleneck (suppression) PCR; library preparation; fragment analysis of products; and sequencing. The additional enrichment step for long-fragment enrichment of certain fragments was performed on the products of the bottleneck (suppression) PCR, and prior to the library preparation step. The fragments, which are products of the suppression PCR, are referred to as ‘long fragments’ to differentiate them from fragments which are the products of the library preparation step and referred to in Example 7 as ‘short fragments’.

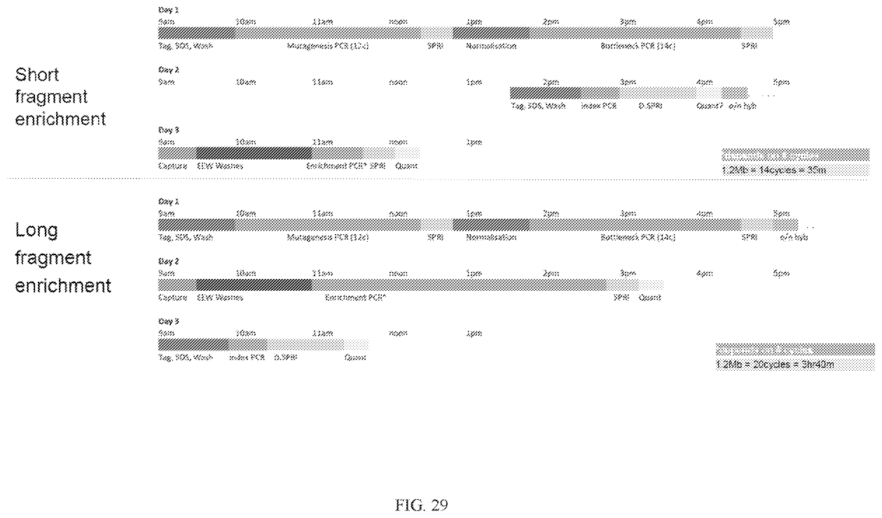

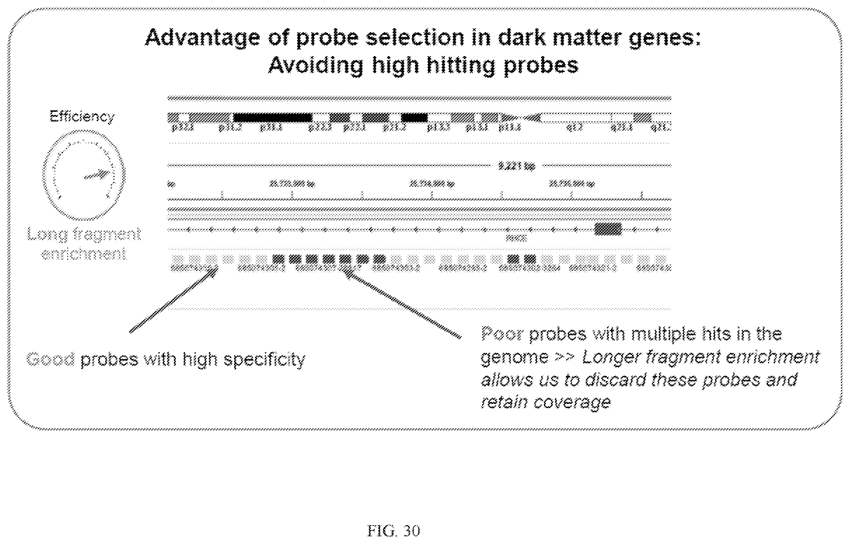

An example overview and timeline for long-fragment enrichment in the workflow is depicted in FIG. 28 and FIG. 29, respectively. The enrichment step included hybridizing the products with selection probes, capturing the products hybridized to the selection probes with bead-linked capture probes, and amplifying the captured products. Example selection probes are disclosed in PCT/US2023/067467; PCT/US2023/067465; PCT/US2023/067466; PCT/US2023/067471; PCT/US2023/067468 which are each incorporated by reference in its entirety. An example protocol is described below in the following. However, it should be realized that other protocols using similar enrichment for long fragments are included within embodiments of the invention.

### Hybridization

Bring DNA libraries (HYB Plate), EHB2, and probes to RT.
Vortex and briefly spin down EHB2 and probes before use.
NOTE: Vortex EHB2 thoroughly and allow enough time to bring up to RT.
Ensure that crystals are fully dissolved. Heat tube in hands if needed.
Preheat NHB2 to 50° C. for 5 m and vortex for 30 sec, ensure solution is clear and free from
precipitate.
NOTE: Vortex NHB2 thoroughly and allow enough time to thaw to RT and get up to 50° C.
prior to use.
Ensure that crystals are fully dissolved. Use while warm to avoid precipitates from reforming.
Prepare Hyb Master Mix and add 17.5 uL to HYB plate wells. (or add components
individually in order: Library, NHB2, Probes, EHB2)
Pipette mix. Seal HYB plate with Microseal ‘B’.
Centrifuge HYB plate briefly at 280 × g for 30 sec.
Place plate on a thermal cycler and run the LCE HYB program overnight. (holds at 58° C.)

| Program name: LCE HYB | | Rxn vol: 25 μl | | Lid: 100 |
|---|---|---|---|---|
| Step | Temp | Duration | | Cycles |
| Hear denture | 98° C. | 5 | min | 1 |
| Ramp Down | 96° C. | −2° | C./min | 19 |
| | 58° C. | | | |
| Hybridize | 58° C. | 30 | min | 1 |

### Capture

Bring SMB3, EEW, EE1, HP3, ET2, and RSB to RT.
Vortex and briefly spin down ET2, EE1 and HP3 before use.
Vortex EEW and SMB3 thoroughly.
Leave the thermal cycler running at 58 C.
Remove the HYB plate from the thermal cycler.
Centrifuge HYB plate at 280 × g for 30 sec.
Vortex SMB3 thoroughly for 1 min before use.
Add 75 μL of SMB3 to each sample.
Pipette mix 20×
Seal HYB wash plate with Microseal ‘B’
Incubate plate on thermal cycler running at 58° C. for 15 min.
Centrifuge HYB wash plate briefly at 280 × g for 30 sec.
Place on a magnetic stand for 2 min.
While on the magnetic stand, use a pipettor set to 100 μL to carefully
remove and discard the supernatant.

### Post-Hybridization Wash

Vortex EEW thoroughly for 1 min before use.
Vortex and briefly spin down EE1 and HP3 before use.

| | |
|---|---|
| PERFORM | Remove the HYB plate from the magnetic stand and add 100 μL of |
| 2X | EEW to each sample. |
| | Pipette mix to fully resuspend the beads. (takes time, make sure all |
| | beads are resuspended) |
| | Seal HYB wash plate with Microseal ‘B’ |
| | Incubate in a thermal cycler at 58° C. for 10 min. |
| | Place HYB plate on a magnetic stand for 2 min. |
| | While on the magnetic stand, use a pipettor to carefully remove and |
| | discard the supernatant. |

Use a P20 pipettor to remove any residual supernatant from the final pellet.
Remove HYB plate from magnet.
Add 100 μL EEW to each well.
Pipette mix to fully resuspend the beads.
Transfer 100 ul of resuspended bead solution to a new PCR plate (prevents reagent carryover).
Seal HYB wash plate with Microseal ‘B’
Place on the thermal cycler at 58° C. for 5 min.
Turn off thermal cycler at 58° C.
Remove from thermalcycler and place on magnetic stand for 2 min or until soln is clear.
Prepare EE1 + HP3 Elution Mix.
Set pipette to 100 μL and discard all supernatant from each well.
Centrifuge at 280 × g for 30 sec then return to magnet for 10 sec.
Use a P20 pipettor to remove any residual supernatant.
Immediately proceed to Elution to prevent excessive drying of beads.

### Elution

Vortex and briefly spin down ET2 before use.
Vortex EE1 + HP3 Elution Mix to mix well.
Remove the HYB plate from the magnetic stand and add 23 μL of EE1 +
HP3 Elution Mix.
Pipette mix to fully resuspend the beads.
Incubate at RT for 2 min.
Centrifuge at 280 × g for 30 sec.
Place on a magnetic stand for 2 min.
Add 4 μL of ET2 to a new 96-well PCR plate to wells corresponding to
sample layout.
Label the plate, “ELU plate”.
Carefully transfer 21 μL of eluate from the HYB wash plate to
corresponding wells of ET2 in the ELU plate.
Set pipette to 20 μL and gently pipette mix each well 10 times.
Centrifuge at 280 × g for 30 sec.

### Enriched Library PCR

Bring MM reagents to RT.
Prepare RED Master Mix. Mix and spin.
Briefly spin down ELU plate to collect condensation if
needed.
Pipette mix.
Seal ELU plate with Microseal ‘B’
Briefly spin down plate to collect droplets.
Place plate on a thermal cycler and run the SYD_EN PCR
program.
(# cycles depends on Probe Panel used)
(same PCR conditions as Bottleneck PCR)

Preprogram the thermocycler with the following
program (heated lid at 105° C.)
Enrichment PCR

| | | |
|---|---|---|
| Step | Temperature | Time |

| | |
|---|---|
| 1 | PCR cycles (depends on panel) |

| | | |
|---|---|---|
| | 98° C. | 10 seconds |
| | 68° C. | 10 minutes |
| 3 | 4° C. | hold |

### PCR Clean Up

Bring IPB and RSB to RT for at least 30 min.
Prepare fresh 80% EtOH wash solution as follows. Vortex to mix well.
Remove ELU plate from thermocycler. Briefly spin down PCR plate to collect droplets.
Vortex IPB to resuspend the beads.
Add 30 ul of IPB beads to each well.
Mix the total reaction volume by gently pipetting at least 10 times and incubate for 2 minutes at
room temperature.
Place on the magnetic stand and wait until the liquid is clear (~5 minutes)
Remove and discard all supernatant
Wash beads as follows: a. Keep on the magnetic stand and add 180 μl fresh 80% EtOH to each
well; b. Wait 30 seconds; and c. Remove and discard all supernatant
Repeat wash for a total of 2 washes.
Briefly spin for 10 seconds and return plate to magnet.
With a 20 μl pipette, remove all residual EtOH.
Dry on magnet for 2 minutes.
Remove from the magnetic stand and add 25 μl RSB to each well.
Pipette mix and Incubate at room temperature for 2 minutes.
Place on the magnetic stand and wait until the liquid is clear (~2 minutes).
Transfer 24 μl supernatant to a new plate.
Keep on ice for same day use or store at −20° c. (SAFE STOPPING POINT).
Determine the concentration of each purified recovery product with the Qubit dsDNA HS Assay
Kit. Use 1-2 μl of sample DNA per measurement.
Proceed with 50 ng to BLT prep (next tab)

### Example 7—Enriching for Short Fragments

A workflow was performed as described in Example 1, but including an enrichment step for short fragments. The workflow included: high molecular weight tagmentation; mutagenesis PCR; library normalization; bottleneck (suppression) PCR; library preparation; fragment analysis of products; and sequencing. The additional enrichment step for short fragment enrichment of certain fragments was performed on the products of the library preparation step. An example overview and timeline for short-fragment enrichment in the workflow are depicted in FIG. 28 and FIG. 29, respectively. Of course, other workflows for such short-fragment enrichment are also contemplated.

The enrichment step included hybridizing the products with selection probes, capturing the products hybridized to the selection probes with bead-linked capture probes, and amplifying the captured products. The enrichment step was substantially the same as that performed in Example 6.

### Example 8—Selection of Probes for Enrichment of Long Fragments

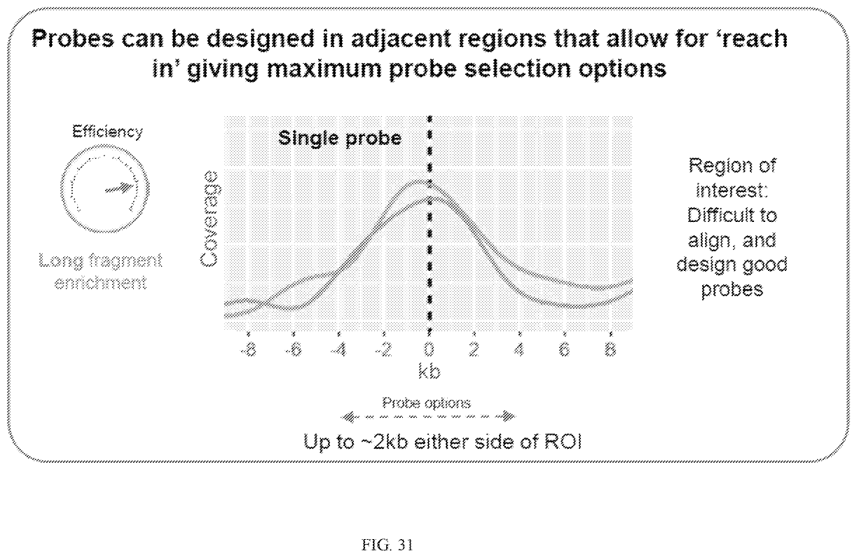

In Example 6, enrichment of products of suppression PCR or ‘long fragments’ by hybridization to selection probes can include selecting the selection probes. As depicted in FIG. 30, selection probes can be selected having increased specificity for a target fragment. For example, selection probes can be selected having sequences that avoid genomic repetitive elements, such as tandem repeats, and interspersed elements, such as Alu repeats, short interspersed nuclear elements (SINEs), long interspersed nuclear elements (LINEs), and integrated viral sequences, such as LTRs and transposons.

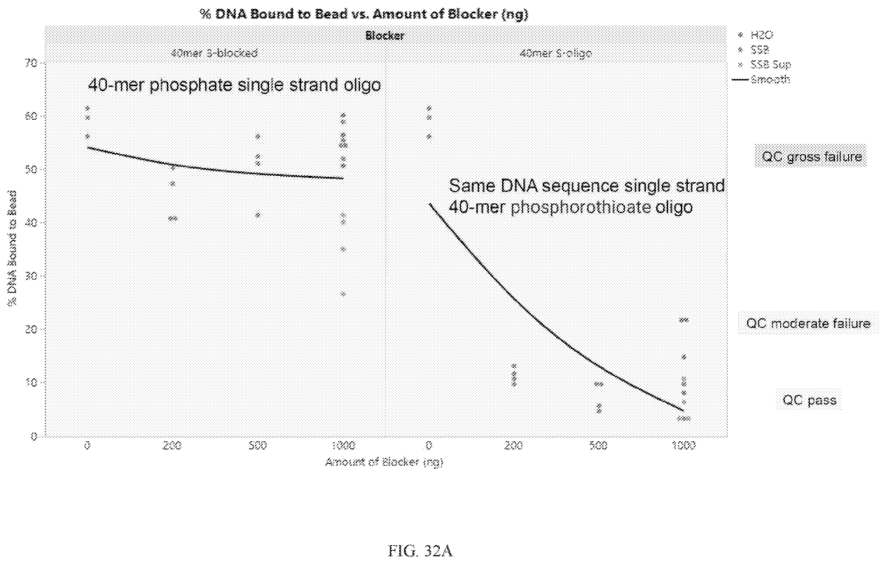

In addition, a workflow that includes an enrichment step for products of suppression PCR or ‘long fragments’, compared to an enrichment step for products of library preparation or ‘short fragments’, can include the use of a fewer number of selection probes because the target fragments are longer. See e.g., FIG. 31.

### Example 9—Reducing Non-Specific Binding to Beads

Some of the methods described herein include the use of bead-linked transposomes in which DNA, such as genomic DNA, is contacted with the transposomes and fragmentated for further downstream library preparing, and sequencing. It was observed that different lots of streptavidin coated magnetic beads had different levels of non-specific binding of DNA. Non-specific binding variation of DNA to different beads lots was undesirable because it led to decreased quality of sequencing metrics.

Oligonucleotides were generated and used to block beads to analyze how they could reduce non-specific binding. Magnetic beads coated with streptavidin were obtained from Cytiva (MA, USA). Oligonucleotides included 20-mers, 40-mers, and 60-mers, and included either a backbone with phosphodiester bonds, or a backbone with phosphorothioate bonds. To reduce the likelihood of the oligonucleotides forming double-stranded structures and substrates for transposomes, sequences were designed to avoid hairpins and other secondary structures that might form at temperatures as low as −40° C. Oligonucleotides included nucleotide sequences having the motif. [AAA(CT)_{X}]_{Y} where X is 2 to 5 repeats, and Y is 2 to 6 repeats. TABLE 1 lists certain oligonucleotides in which the polynucleotide backbone included either phosphodiester bonds or phosphorothioate bonds.

TABLE 1

| Oligo | Sequence | SEQ ID NO | Backbone |
|---|---|---|---|
| 60-mer | AAACTCTCTCTCTAAACTCT | SEQ ID NO: 02 | Phosphodiester |
| P-oligo | CTAAACTCTCTAAACTCTCT | | bonds |
| | AAACTCTCTCTCTAAACTCT | | |
| 60-mer | AAACTCTCTCTCTAAACTCT | SEQ ID NO: 02 | Phosphorothioate |
| S-oligo | CTAAACTCTCTAAACTCTCT | | bonds |
| | AAACTCTCTCTCTAAACTCT | | |
| 40-mer | AAACTCTCTCTCTAAACTCT | SEQ ID NO: 03 | Phosphodiester |
| P-oligo | CTAAACTCTCTAAACTCTCT | | bonds |
| 40-mer | AAACTCTCTCTCTAAACTCT | SEQ ID NO: 03 | Phosphorothioate |
| S-oligo | CTAAACTCTCTAAACTCTCT | | bonds |
| 20-mer | AAACTCTCTCTCTAAACTCT | SEQ ID NO: 04 | Phosphodiester |
| P-oligo | | | bonds |
| 20-mer | AAACTCTCTCTCTAAACTCT | SEQ ID NO: 04 | Phosphorothioate |
| S-oligo | | | bonds |

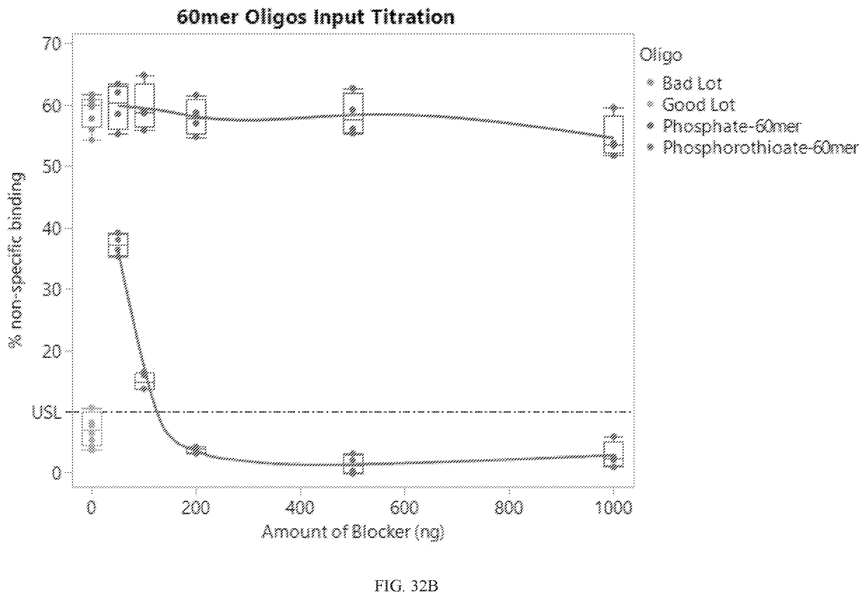

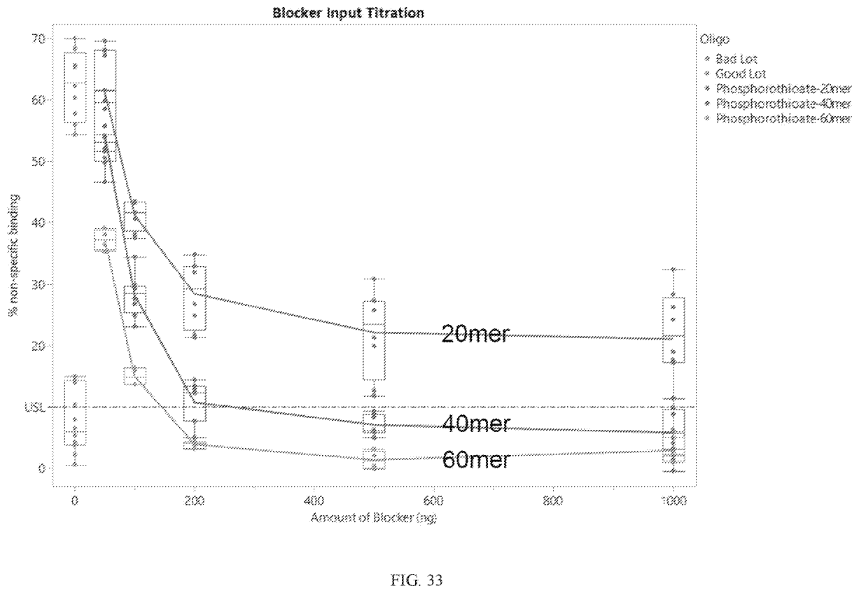

A bead lot was selected which had been shown to have high levels of non-specific DNA binding. Beads were blocked with increasing amounts of the 40-mer oligonucleotides, and the percentage of genomic DNA bound to the beads was measured. As shown in FIG. 32A, beads blocked with the 40-mer phosphorothioate oligo (S-oligo) had significantly lower levels of non-specific DNA binding compared to beads blocked with the 40-mer phosphodiester single strand oligo (P-oligo).

Levels of non-specific DNA blocking on beads were compared for the 60-mer S-oligo and 60-mer P-oligo. Levels of non-specific DNA binding on beads were compared for after using the 60-mer S-oligo and 60-mer P-oligo for blocking, respectively. Beads lots were tested that had been shown to have either high or low levels of non-specific DNA binding. As shown in FIG. 32B, beads blocked with the 60-mer S-oligo had significantly lower levels of non-specific DNA binding compared to beads blocked with the 60-mer P-oligo.

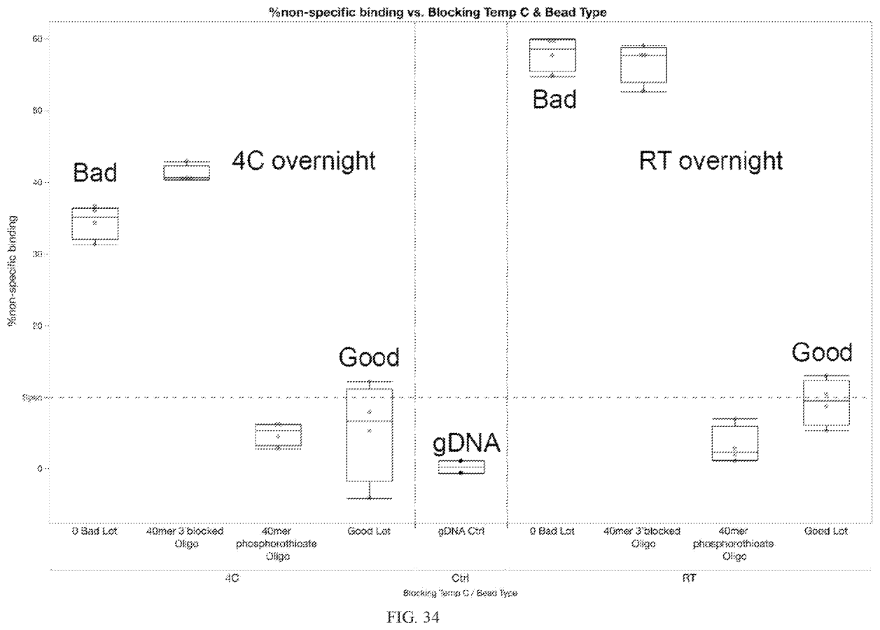

Levels of non-specific DNA blocking on beads were compared for S-oligos of different lengths. Levels of non-specific DNA binding on beads were compared for after using for blocking, respectively. Beads lots were tested that had been shown to have either high or low levels of non-specific DNA binding. Beads were blocked overnight at 4° C. As shown in FIG. 33, S-oligos having a longer length were more effective at blocking non-specific DNA binding to beads than shorter S-oligos.

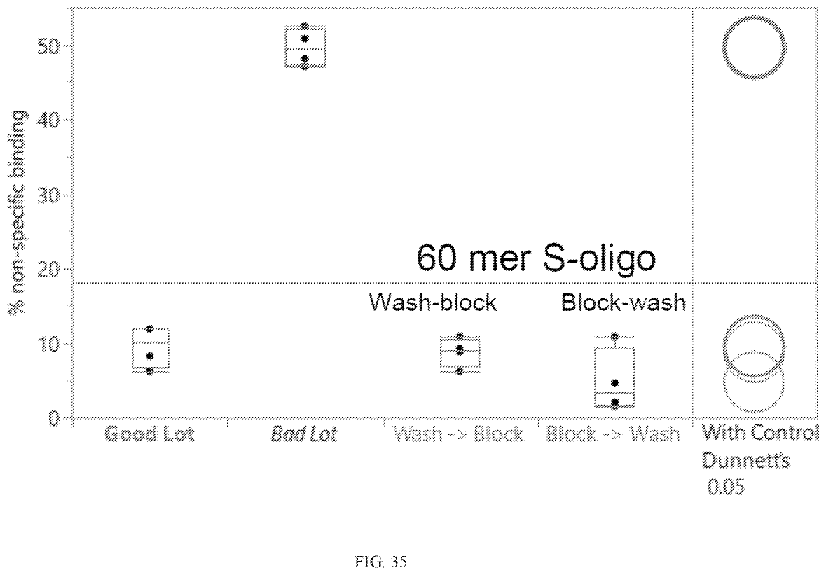

Levels of non-specific DNA blocking on beads were compared by incubating 40-mer oligonucleotides with beads overnight at either room temperature or at 4° C. Beads lots were tested that had been shown to have either high or low levels of non-specific DNA binding. As shown in FIG. 34, the temperature at which beads were incubated with oligonucleotides had no significant effect on the effectiveness of the 40-mer S-oligo to reduce non-specific DNA binding.

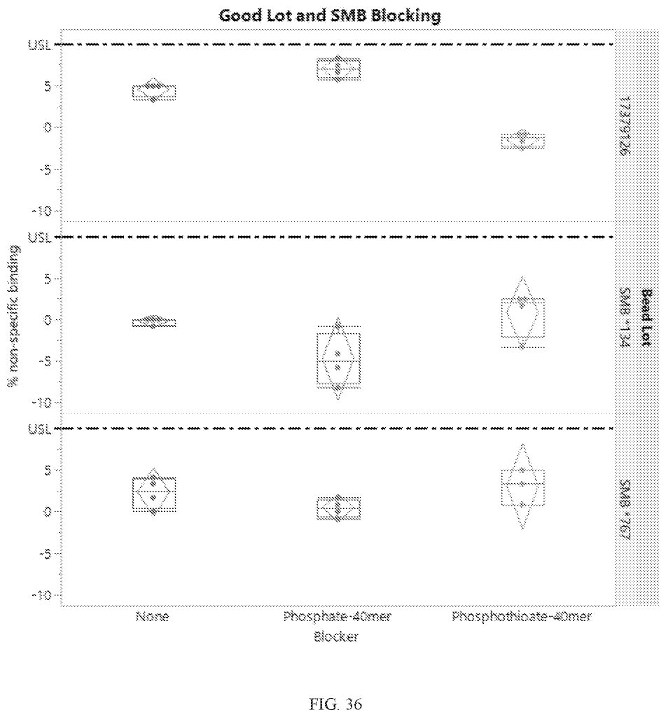

Levels of non-specific DNA blocking on beads were compared for beads that were either (i) washed, then blocked with 60-mer S-oligo, and genomic DNA non-specific binding tested; or (ii) blocked with 60-mer S-oligo, then washed, and genomic DNA non-specific binding tested. As shown in FIG. 35, the order of blocking and washing the beads had no significant effect on the effectiveness of the 60-mer S-oligo to reduce non-specific DNA binding.

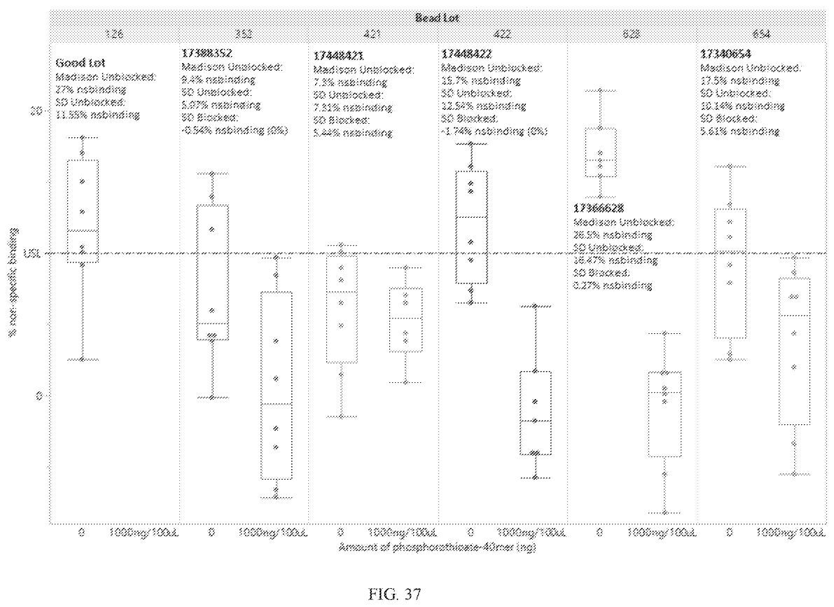

Levels of non-specific DNA blocking on beads were compared by incubating 40-mer S oligo or 40-mer P-oligo with a bead lot that had been shown to have low levels of non-specific DNA binding. As shown in FIG. 36, blocking beads that had been shown to have low levels of non-specific DNA binding with 40-mer S oligo was not detrimental. Thus, S-oligo blocking may be applied uniformly with bead lots that had either high or low levels of non-specific DNA binding.

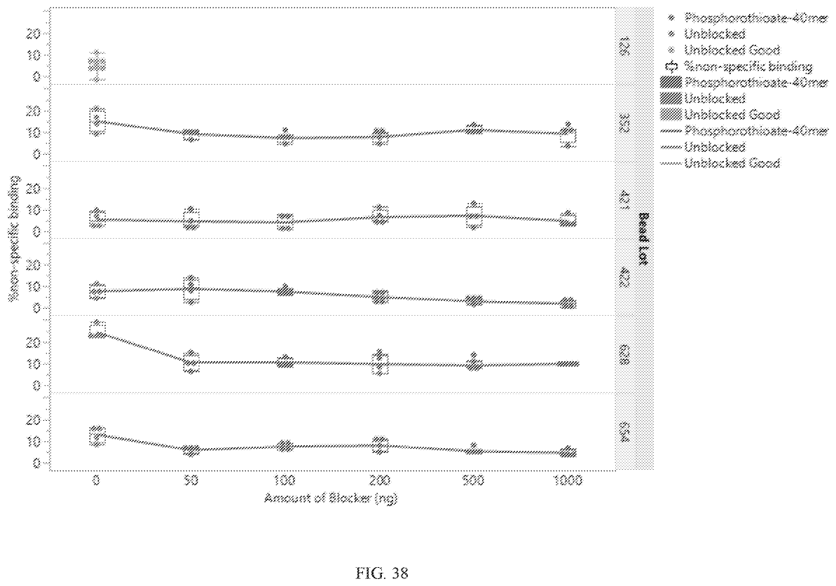

Levels of non-specific DNA blocking on beads were compared by incubating 40-mer S oligo with various lots of beads that had been shown to have high levels of non-specific DNA binding. As shown in FIG. 37, a 40-mer S oligo was capable of reducing non-specific DNA binding in various bead lots that had been shown to have high levels of non-specific DNA binding. Thus, lot to lot variation for levels of non-specific DNA binding may be minimized by blocking with S oligos.

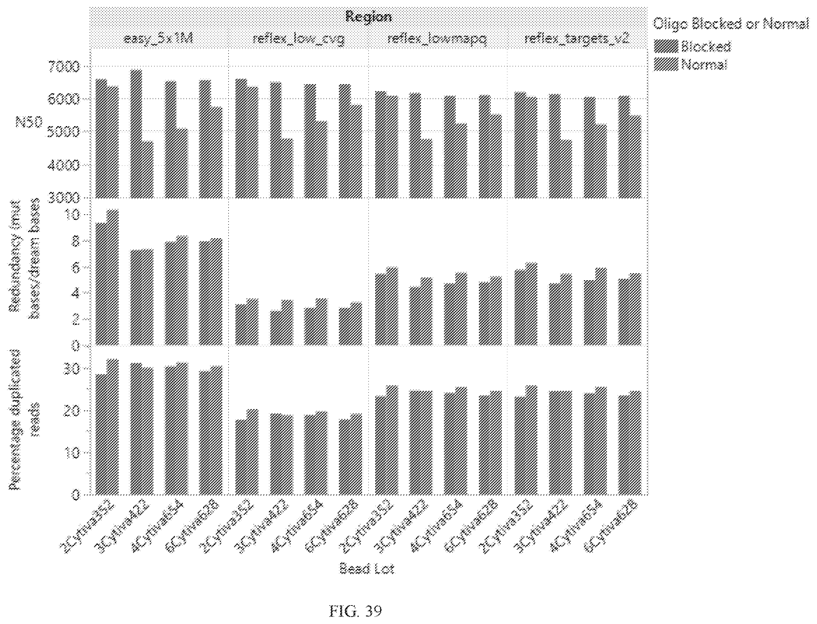

Levels of non-specific DNA blocking on beads were compared by incubating various amounts of 40-mer S oligo with various lots of beads. As shown in FIG. 38, levels of non-specific DNA blocking on beads were substantially the same after contacting the beads with a certain amount of the blocker.

### Example 10—Improved Sequencing Metrics with Blocked Beads

Effects of blocking beads with the 40-mer S-oligo was tested for a protocol substantially similar to the workflow of Example 1.

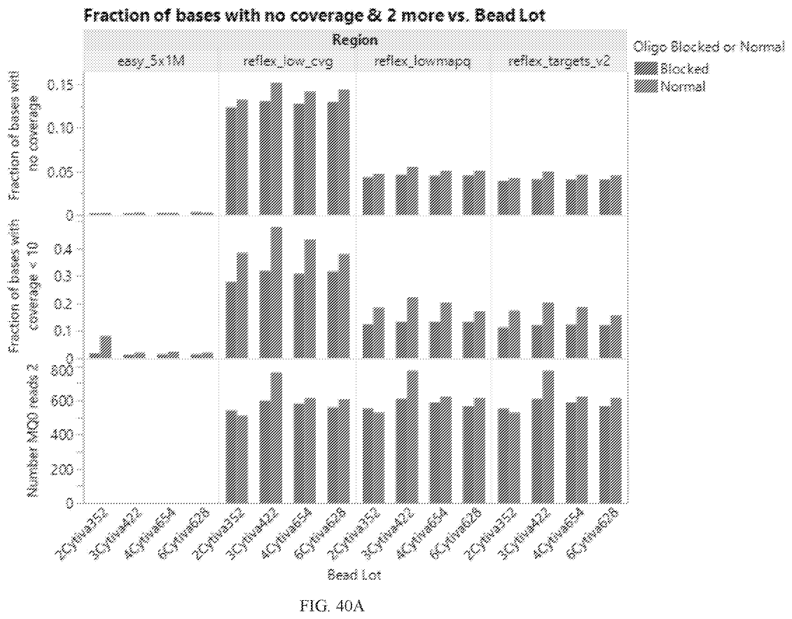

Sequence metrics were evaluated for sequences obtained from different genomic regions using the workflow in which beads were blocked or not blocked. Sequencing metrics included an N50 value, levels of redundancy, and percentage duplicated reads. The N50 measured the bioinformatically assembled medium length of DNA fragments using the long-read sequencing workflow. The N50 was a quality metric and was used in providing DNA fragment sequences as building blocks into a final long-read sequencing assembly. Generally, longer and consistent N50 values were indicative of better final long-read sequencing assembly. For the long-read sequencing workflow, use of blocked beads provided improved consistency in N50 metrics, longer reads, and minimized effects of variation between bead lots. FIG. 39 shows sequencing metrics including those for N50, redundancy, and percentage duplicates, obtained using a workflow with blocked or not blocked beads, for various genomic regions. Use of blocked beads improved N50 values' uniformity.

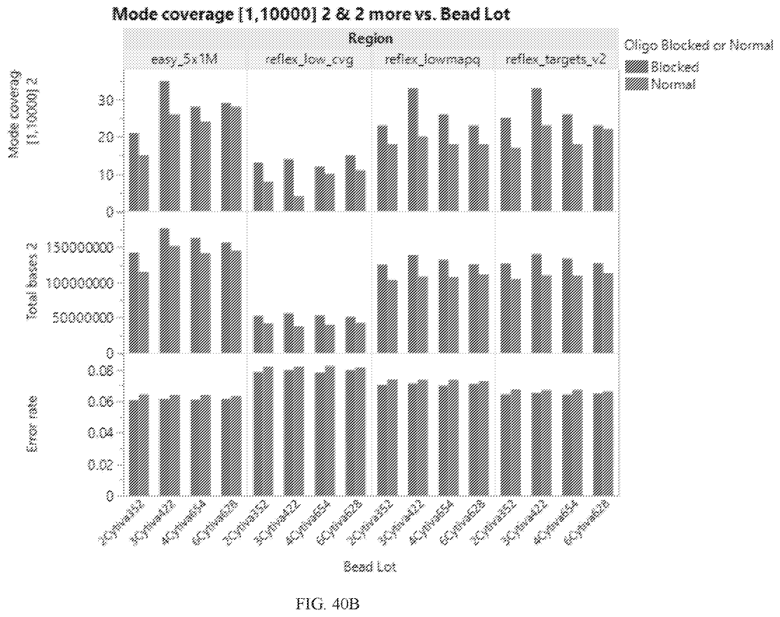

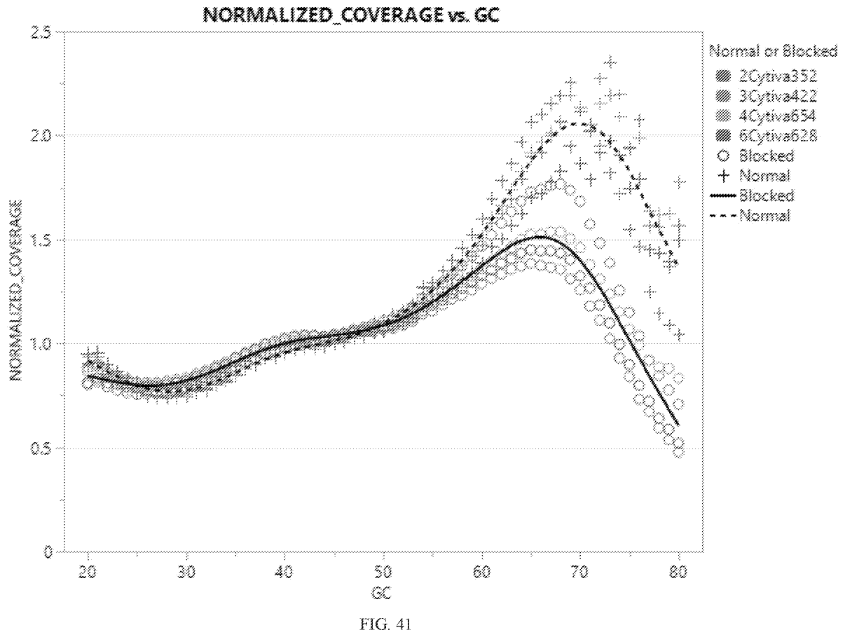

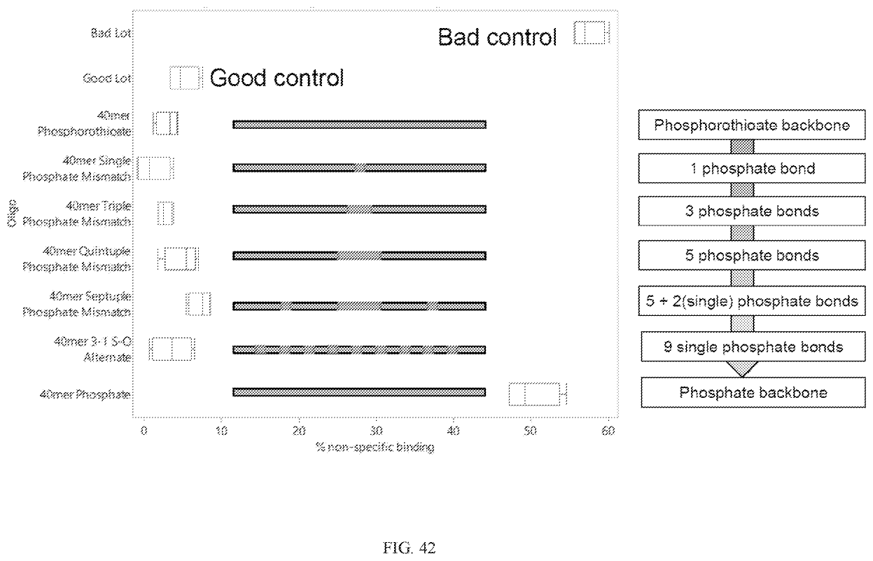

Blocked and not blocked beads from various bead lots provided substantially similar levels of yield (ng/ul) and fragment size (bp) throughout the steps of the workflow, including post-mutagenesis, post-normalization, post-bottleneck, and for final library preparation. As shown in FIG. 40A and 40B, the fraction of bases with no coverage in a region was lower, and the mode coverage was improved for a workflow that included blocked beads compared to not blocked beads, respectively. As shown in FIG. 41, an improved GC bias was obtained for a workflow that included blocked beads compared to not blocked beads.

### Example 11—Oligonucleotides with Partial Phosphorothioate Backbones

Oligonucleotides with a phosphorothioate backbone were generated that included one or more substitutions of a phosphorothioate bond with a phosphodiester bond. The following TABLE 2 lists positions of the substitutions.

TABLE 2

| Oligo | Sequence | SEQ ID NO |
|---|---|---|
| 40 mer 3-1 | A*A*A*CT*C*T*CT*C*T*CT*A*A*A | SEQ ID |
| S-O | C*T*C*TC*T*A*AA*C*T*CT*C*T*A | NO: 03 |
| alternate | A*A*C*TC*T*C*T | |
| 40 mer | A*A*A*C*T*C*T*C*T*C*T*C*T*A* | SEQ ID |
| Single | A*A*C*T*C*TC*T*A*A*A*C*T*C* | NO: 03 |
| Phosphate | T*C*T*A*A*A*C*T* C*T*C *T | |
| Mismatch | | |
| 40 mer | A*A*A*C*T*C*T*C*T*C*T*C*T*A* | SEQ ID |
| Triple | A*A*C*T*CTCT*A*A*A*C*T*C*T* | NO: 03 |
| Phosphate | C*T*A*A*A*C*T*C*T*C*T | |
| Mismatch | | |
| 40 mer | A*A*A*C*T*C*T*C*T*C*T*C*T*A* | SEQ ID |
| Quintuple | A*A*C*TCTCTA*A*A*C*T*C*T*C* | NO: 03 |
| Phosphate | T*A*A*A*C*T*C*T*C*T | |
| Mismatch | | |
| 40 mer | A*A*A*C*T*C*T*C*TC*T*C*T*A* | SEQ ID |
| Septuple | A*A*C*TCTCTA*A*A*C*T*C*T*C*T | NO: 03 |
| Phosphate | A*A *A*C*T*C*T*C*T | |
| Mismatch | | |

*denotes position of a phosphorothioate bond in the polynucleotide backbone.
Underline denotes bases joined via a phosphodiester bond.

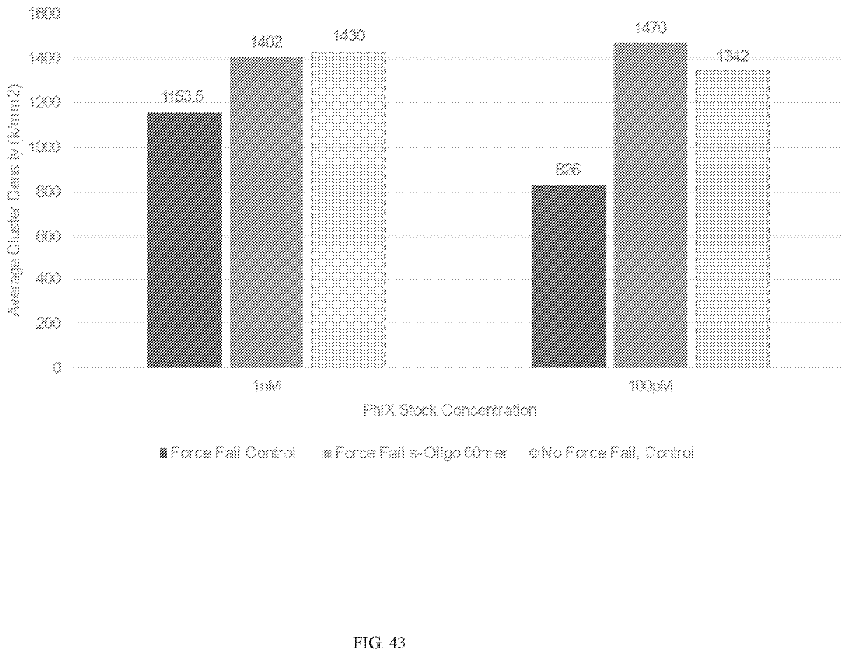

Beads were incubated with a saturating amount of oligos (500 ng) for 2 hours at room temperature, and the percentage of non-specific DNA binding was measured, As shown in FIG. 42, increasing the number of phosphodiester bonds in the S-oligos increased the level of non-specific DNA binding to the beads.

### Example 12—Use of S-Oligomer Protected against Freeze Thaw Cycling

A study was performed in which 1 nM or 100 pM PhiX samples underwent 6 freeze-thaw cycles in the presence or absence of the S-oligo 60-mer. The S-oligo 60-mer is listed in TABLE 1. A control included a PhiX sample in which no freeze-thaw cycles were performed.

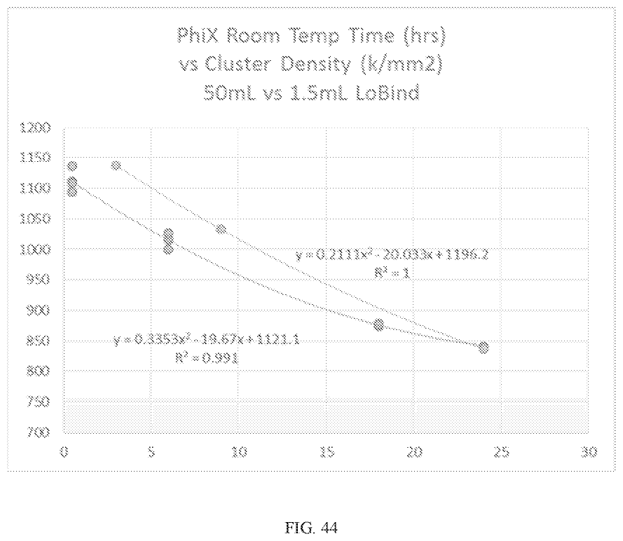

Samples underwent six freeze thaw cycles with 5 days at −20° C., and 1 hour at room temperature. Samples were then incubated at room temperature for 24 hours, and sequenced. FIG. 43 shows that the average cluster density was greater for 1 nM and 100 pM PhiX samples that included the S-oligo 60-mer (force fail S-oligo 60mer) compared to samples that did not include the S-oligo 60-mer (force fail control). In addition, the average cluster density was also greater for 100 pM PhiX samples that included the S-oligo 60-mer (force fail S-oligo 60mer) compared to 100 pM PhiX samples that did not undergo freeze-thaw cycles (no force fail, control). The S-oligo 60-mer protected against the effects of freeze thaw cycles, and also increased sequencing efficiency at low concentrations of PhiX.

### Example 13—Sequencing Data from Different Flow Cells

This example relates to the use of a large sample size with high diversity to understand sources of variation in sequencing runs. The study included more than 300 sequencing runs performed on the NOVASEQ™ (Illumina, Inc., San Diego) sequencing platform. The samples for the sequencing runs included different lots of SBS reagents, CPE reagents, buffer reagents, PhiX control, and flow cells. At least 10 replicate runs were performed for each lot of reagents. Each sequencing run was performed on a new machine. All samples were loaded with a control nucleic acids, PhiX, following the standard end-user protocol. Traditionally, the primary metrics such as intensity and Q30 were not thought to be correlated with the flow cell primer lawn density, as measured through the QC method of CFR. In this study, the flow cell variable was isolated while holding the PhiX lot constant. This revealed a linear correlation of primer density and sequencing intensity. This correlation varied across flow cell lots as expected, due to the variation in primer lawn density accessibility across lots. When isolating for the correct variables, we confirmed that the linear correlation existed for primer density and intensity within a flow cell lot (while holding the largest source of variation constant, the PhiX lot). The correlation showed that the clustering chemistry had a strong dependency to loading concentration and primer lawn density. This finding illustrated a dependency of the PhiX concentration from batch to batch, illuminating a mechanism and need for the reduction in variation in PhiX concentration.

Different lots of PhiX control nucleic acids were loaded on to a NOVASEQ™ (Illumina, Inc., San Diego) sequencing platform. Cal flour red (CFR) dye intensities and sequencing intensities were measured for each lot. The CFR metric was a dye based hybridization assay for the primer density on the flow cell. It was found that sequencing intensity correlated with CFR within flow lots, but sequencing intensity was different between lots.

### Example 14—Solid Phase Nucleic Acid Amplification in the Presence of Carrier DNA

Effects of spiking samples with carrier DNA were tested using: a MINISEQ™ (Illumina, Inc., San Diego) sequencing platform which included bridge amplification on an unpatterned substrate; a NOVASEQ™ (Illumina, Inc., San Diego) sequencing platform which included exclusion amplification on a patterned substrate without onboard denaturation; and a NEXTSEQ 1k/2k™ (Illumina, Inc., San Diego) sequencing platform which included bridge amplification on a patterned substrate with onboard denaturation.

A PhiX control sample was denatured and neutralized for each platform, and carrier DNA (salmon sperm DNA, INVITROGEN) was added to the ready to load PhiX control sample. The following TABLE 3 lists QScore distribution values for sequencing runs on various platforms.

TABLE 3

| | QScore distribution (>=Q30) | |
|---|---|---|
| | Control sample without | Test sample with carrier |
| Platform | carrier DNA | DNA |
| NextSeq 1k/2k | 93.2% | 93.4% |
| NovaSeq | 93.8% | 91.8%; |
| MiniSeq | 97.3% | 98.1% |

Each platform showed no significant effect of adding the carrier DNA to the prepared sequencing samples compared to controls without carrier DNA.

### Example 15—Effects of Carrier DNA on Nucleic Acid Samples Post-Denaturation

Before a sequencing run, a nucleic acid sample is denatured either with NaOH or heat to separate strands. The samples are then either neutralized or allowed to start cooling, and the strands of the nucleic acid sample may then begin to anneal to one another forming secondary structures, and also begin to tangle and form tertiary structures. Thus, prepared samples can have a time sensitivity as annealing and tangling continues which may result in reduced sequencing efficiencies.

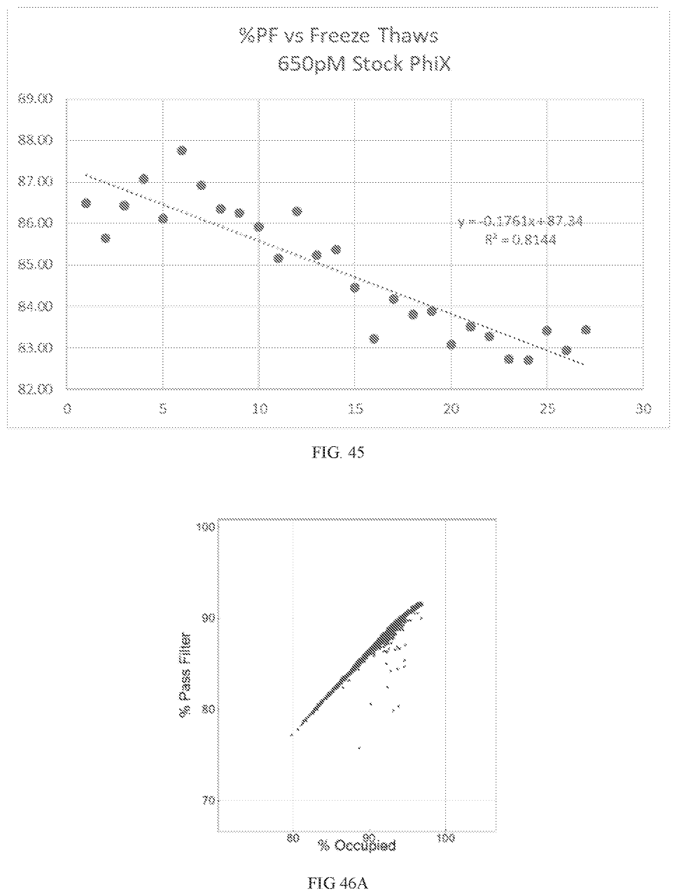

The following experiment relates to reduced sequencing efficiencies as nucleic acid samples are incubated at room temperature for increased periods. 700 μl 10 pM PhiX library samples in 1.5 mL tubes were prepared for the MISEQ™ (Illumina, Inc., San Diego) sequencing platform. After denaturation with NaOH and neutralization, samples were incubated at room temperature for various times. Incubated samples were sequenced on the platform. As shown in FIG. 44, there was a decay in measured cluster density for samples that had been held at room temperature for a longer period (lower line). In FIG. 44, the upper line relates to samples that had been incubated in a 50 ml tube.

Different sequencing platforms have different time sensitivities for sample loading after sample denaturation. For example, sequencing platforms with unpatterned substrates, such as those which may include bridge amplification (e.g., MISEQ™, NEXTSEQ 550™, MINISEQ™, HISEQ 2000™, all Illumina, Inc., San Diego), can include nucleic acid sample denaturation with NaOH followed by neutralization, prior to loading the sample. Such platforms have an increased time sensitivity for sample loading compared to other platforms. Sequencing platforms with patterned substrates, such as those which include exclusion amplification, include the use of enzymes which separate annealed strands but may not efficiently de-tangle nucleic acids. Sequencing platforms with patterned substrates and without onboard denaturation (e.g., HISEQX™, NOVASEQ™, NOVASEQX™, all Illumina, Inc., San Diego) have a decreased time sensitivity for sample loading compared platforms with unpatterned substrates. Sequencing platforms with patterned substrates and with onboard denaturation (e.g., ISEQ™, NEXTSEQ 2K™, FUTURE™, all Illumina, Inc., San Diego) have an even greater decreased time sensitivity for sample loading compared platforms with patterned substrates and without onboard denaturation.

The following experiments relate to testing sequencing efficiencies of nucleic acid samples incubated at room temperature on different sequencing platforms. A series of experiments were carried on the following sequencing platforms: MISEQ™, NOVASEQ™, ISEQ™, NEXTSEQ 2K™ (all Illumina, Inc., San Diego). Platforms were loaded and run with either: 200 pM PhiX nucleic acid sample from −80° C. (control sample), or 200 pM PhiX nucleic acid sample incubated at room temperature for 24 hours (test sample). Sequencing efficiencies were most reduced for test samples run on the MISEQ™ (Illumina, Inc., San Diego) platform; less reduced for test samples run on the NOVASEQ™ (Illumina, Inc., San Diego) platform; and no substantial reduction for test samples run on the ISEQ™ and NEXTSEQ 2K™ (both Illumina, Inc., San Diego) platforms.

The following experiment relates to testing sequencing efficiencies of nucleic acid samples including carrier DNA, and incubated at room temperature on different sequencing platforms. Non-denatured samples of 100 pM PhiX (control), 100 pM PhiX with 1 pM carrier DNA (test) were incubated at room temperature for 24 hours, then loaded on to either an ISEQ™ or NEXTSEQ 2K™ (both Illumina, Inc., San Diego) platform. The following TABLE 4 lists certain metrics related to sequencing efficiencies for samples run on each platform. Sequencing efficiencies for samples with or without carrier DNA were substantially the same for each platform.

TABLE 4

| | Nucleic | Sequencing metric | | | | |
|---|---|---|---|---|---|---|
| | acid | % >= | Clusters | Aligned | Error | |
| Platform | sample | Q30 | PF (%) | (%) | rate (%) | CFR |
| NEXTSEQ | control | 95.57 | 76.44 ± 9.73 | 98.10 ± 12.21 | 0.05 ± 0.07 | 2,003.00 |
| 2K ™ | test | 94.23 | 77.37 ± 9.98 | 97.97 ± 12.22 | 0.07 ± 0.11 | 2,002.50 |
| ISEQ ™ | control | 95.4 | 78.89 ± 1.85 | 99.25 ± 0.12 | 0.12 ± 0.02 | 261 |
| | test | 94.32 | 73.10 ± 11.48 | 95.96 ± 9.01 | 0.14 ± 0.05 | 266 |
| | control | 95.21 | 78.46 ± 1.34 | 99.20 ± 0.11 | 0.12 ± 0.01 | 223 |
| | test | 95.19 | 76.13 ± 0.91 | 99.17 ± 0.12 | 0.12 ± 0.01 | 221 |

### Example 16—Reducing Sample Variation

Sequencing efficiencies of different samples of PhiX control nucleic acid can vary. Therefore, an end-user may need to titrate the PhiX control nucleic acid for use on a sequencing platform.

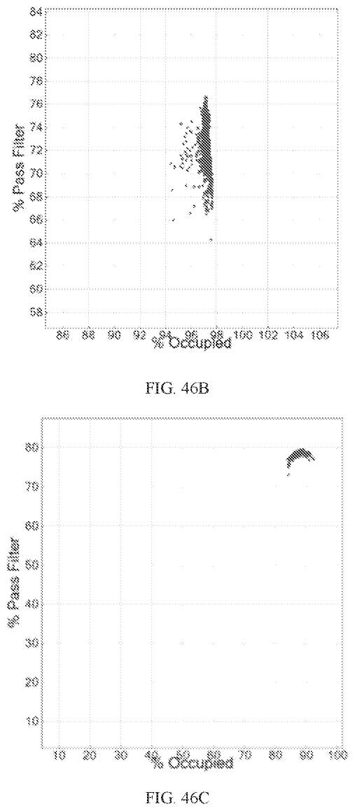

A study was performed in which 650 pM PhiX underwent several freeze-thaw cycles. Samples were loaded on a NEXTSEQ 2K™ (Illumina, Inc., San Diego) platform and sequencing efficiencies of the samples were determined. As shown in FIG. 45, sequencing efficiencies as measured by percentage pass filter (% PF) were reduced as the number of freeze-thaw cycles increased.

Another study was performed in which a 10× concentration of PhiX underwent 6 freeze-thaw cycles. Sequencing efficiencies as measured by percentage pass filter (% PF) were substantially the same for control and test samples. Briefly, to illustrate a protective effect of higher concentrations of PhiX during events, such as shipping and handling, a force fail experiment was performed in which 10 nM or 100 nM PhiX underwent six freeze-thaw cycles. Experiments were performed on 2 MISEQ™ (Illumina, Inc., San Diego) sequencing platform using the same PhiX lot, same reagent and buffer lots. Samples underwent 6× freeze thaw cycles with 5 days at −20° C., and 1 hour at room temperature. Samples were then incubated at room temperature for 24 hours. The force failed test condition vs a control condition (new tube) was run on each machine to show the differences in key metrics including pass filter (PF) and Q30. Freeze-thaw samples with a concentration of 10 nM showed a decay of ˜10% PF compared to control. Freeze-thaw samples with a concentration of 100 nM showed no decay as measured by metrics including PF. These results showed that lower concentration DNA samples were detrimentally sensitive to conditions that may occur during repeated freeze thaw conditions.

A study was performed to compare sequencing runs with control PhiX samples that had been either removed directly from storage conditions at −80° C. or test PhiX samples that included carrier DNA and had been incubated at room temperature for 24 hours. Two sets of experiments were performed on ISEQ™ or NEXTSEQ 2K™ (all Illumina, Inc., San Diego) sequencing platforms. The sequencing runs had identical cartridge lots, flow cell lots, and PhiX lots. The PhiX tubes were prepped at the ready to load concentrations and configurations before the testing started. Four sequencing runs were run for each platform. Control samples included fresh PhiX; test samples included PhiX and carrier DNA incubated at room temperature for 24 hours. In both experiments, the test samples did not show a difference in sequencing metrics from the control PhiX samples.

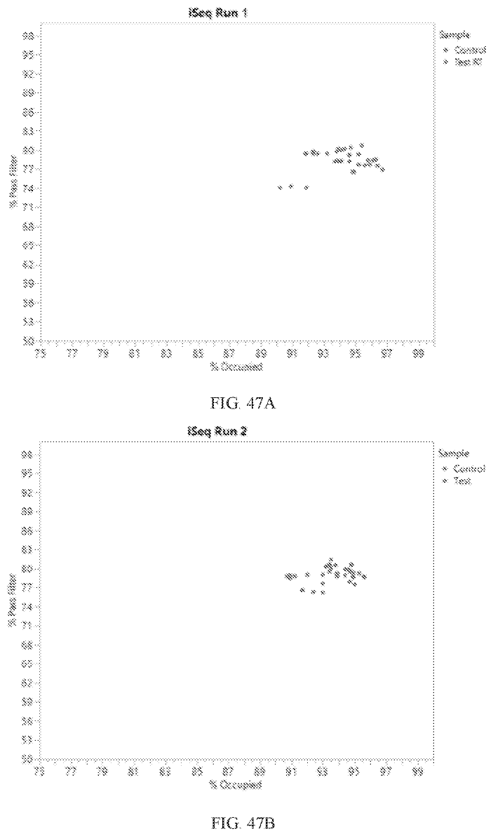

FIG. 46A, FIG. 46B and FIG. 46C depict examples plots for sequencing runs performed on a patterned flowcell in which samples were unloaded, overloaded, or optimally loaded. For underloaded samples, a % PF filter vs % occupied plot shows points which fall on a line with a positive slope from the bottom left to the top-right of the plot (FIG. 46A. For overloaded samples, a % PF filter vs % occupied plot shows points which have a slightly negative near vertical slope and approach a percentage occupied in the high 90s (FIG. 46B). For optimally loaded samples, a % PF filter vs % occupied plot shows points which fall within a cloud of points with a positive slope in the body of the plot (FIG. 46C).

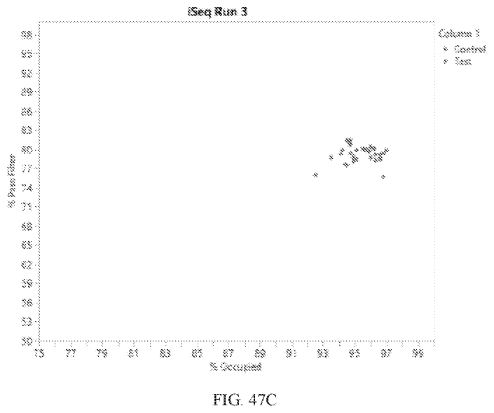

FIG. 47A, FIG. 47B and FIG. 47C depict three plots for sequencing runs performed on an ISEQ™ (Illumina, Inc., San Diego) sequencing platform with control samples that included fresh 60 pM PhiX; and test samples which included 60 pM PhiX and carrier DNA incubated at room temperature for 24 hours. Control and test samples showed substantially the same sequencing metrics.

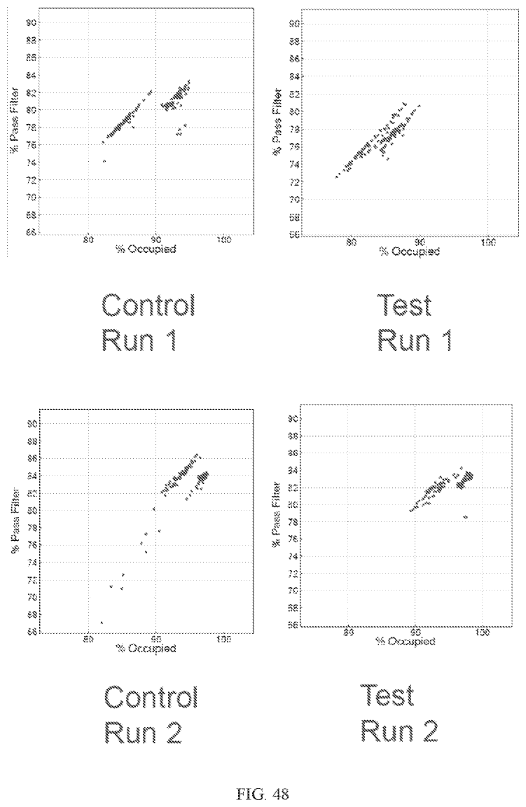

FIG. 48 depicts an additional two plots for sequencing runs with control samples that included fresh 200 pM PhiX; and test samples which included 200 pM PhiX and carrier DNA incubated at room temperature for 24 hours. Control and test samples showed substantially the same sequencing metrics.

### Example 17—Use of Carrier DNA Protected against Freeze Thaw Cycling

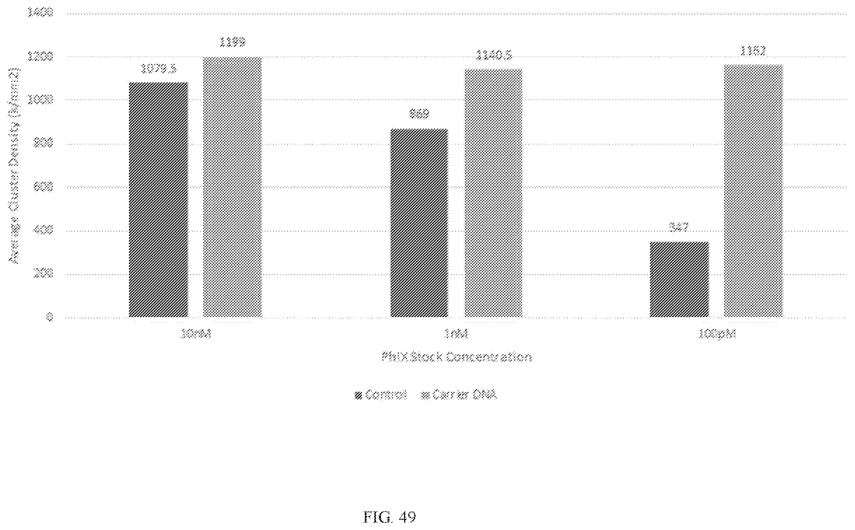

A study was performed in which 10 nM, 1 nM or 100 pM PhiX samples underwent 6 freeze-thaw cycles in the presence or absence of carrier DNA. Samples underwent 6× freeze thaw cycles with 5 days at −20° C., and 1 hour at room temperature. PhiX Stock was obtained from −80° C. Carrier DNA was spiked in at 1:100. Samples were sequenced. FIG. 49 shows that the average cluster density was greater for all PhiX samples that included carrier DNA compared to samples that did not include carrier DNA (control). In addition, the average cluster density was significantly greater for 100 pM PhiX samples that included carrier DNA compared to PhiX samples lacking carrier DNA. Freeze thaw data confirmed carrier DNA protected against cluster density decay in lower concentrated PhiX samples.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it covers all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1-115. (canceled)

116. A method for reducing non-specific nucleic acid binding to a substrate, or normalizing a level of non-specific nucleic acid binding to the substrate, comprising:
contacting the substrate with an oligonucleotide, wherein the oligonucleotide comprises a backbone comprising a phosphorothioate bond, and wherein non-specific nucleic acid binding to the substrate is reduced compared to a substrate not contacted with the oligonucleotide, wherein the oligonucleotide comprises one or more of:
(i) at least 50% of the backbone comprises phosphorothioate bonds;
(ii) a length greater than 20 consecutive nucleotides;
(iii) a sequence lacking the capability of forming a hairpin structure at a temperature less than 25° C.;
(iv) a nucleotide sequence motif of [AAA(CT)_{X}]_{Y}, wherein X is 2 to 5, and Y is 2 to 6; and
(v) the oligonucleotide is single-stranded.

117. The method of claim 116, wherein the oligonucleotide comprises at least 90% sequence identity to the nucleotide sequence set forth in any one of SEQ ID NOs: 02-04.

118. The method of claim 116, wherein the substrate comprises a bead.

119. The method of claim 116, further comprising contacting a plurality of substrates with a plurality of the oligonucleotides, wherein the plurality of substrates comprises beads from different lots.

120. A blocked bead composition comprising a magnetic bead in contact with an oligonucleotide, wherein the oligonucleotide comprises a backbone comprising a phosphorothioate bond, and wherein non-specific nucleic acid binding to the blocked bead is reduced compared to non-specific nucleic acid binding to a bead not in contact with the oligonucleotide, wherein the oligonucleotide comprises one or more of:
(i) at least 50% of the backbone comprises phosphorothioate bonds;
(ii) a length greater than 20 consecutive nucleotides;
(iii) a sequence lacking the capability of forming a hairpin structure at a temperature less than 25° C.;
(iv) a nucleotide sequence motif of [AAA(CT)_{X}]_{Y}, wherein X is 2 to 5, and Y is 2 to 6; and
(v) the oligonucleotide is single-stranded.

121. The blocked bead composition of claim 120, wherein the oligonucleotide comprises at least 90% sequence identity to the nucleotide sequence set forth in any one of SEQ ID NOs: 02-04.

122. A method for preparing a nucleic acid library, comprising:
(a) obtaining a plurality of transposomes comprising transposon adaptors, wherein the plurality of transposomes are immobilized on the blocked bead composition of claim 120;
(b) contacting a plurality of nucleic acid fragments with the plurality of transposomes to obtain a plurality of polynucleotides;
(c) amplifying the plurality of polynucleotides to obtain amplified polynucleotides; and
(d) adding library adapters to each end of the amplified polynucleotides, thereby obtaining the nucleic acid library.

123. The method of claim 122, wherein step (c) comprises contacting the plurality of polynucleotides with a plurality of blocked bead compositions.

124. The method of 122, wherein the plurality of the transposomes is immobilized on the bead at a density such that: (i) an average length of the plurality of polynucleotides greater than about 1 kbp; (ii) the number of transposomes immobilized on the bead is no more than about 100 transposomes; (iii) the bead comprises a total activity such that an average length of the plurality of polynucleotides greater than about 1 kbp; and/or (iv) the bead comprises an activity in a range from about 0.05 AU/μl to about 0.25 AU/μl.

125. The method of claim 122, wherein the transposon adapters comprise the same sequence; and/or the transposomes of the plurality of transposomes are the same.

126. The method of claim 122, wherein the step (c) comprises a mutagenesis PCR, such that mutations are introduced into amplified polynucleotides.

127. The method of claim 122, wherein: (i) a first end of a polynucleotide of the plurality of polynucleotides is capable of annealing to a second end of the polynucleotide of the plurality of polynucleotides; (ii) a first end of an amplified polynucleotide is capable of annealing to a second end of the amplified polynucleotide; and/or (iii) the amplified polynucleotides have an average length greater than about 1 kbp.

128. The method of claim 122, wherein step (c) further comprises a suppression PCR.

129. The method of claim 128, wherein the suppression PCR comprises use of a single amplification primer, and the suppression PCR comprises no more than 16 cycles.

130. The method of claim 122, further comprising enriching for target nucleic acids in the amplified polynucleotides by hybridizing a plurality of selection probes with the amplified polynucleotides, wherein the plurality of selection probes is capable of specifically hybridizing with the target nucleic acids.

131. The method of claim 122, wherein step (d) comprises contacting the amplified polynucleotides with an additional plurality of transposomes.

132. The method of claim 122, wherein an amount of the plurality of nucleic acid fragments is less than about 100 ng.

133. The method of claim 122, further comprising:
sequencing the library of nucleic acids to obtain sequence reads; and
assembling sequence reads to obtain the sequence of a target nucleic acid.

134. The method of claim 122, further comprising enriching for target nucleic acids in the amplified polynucleotides or for target polynucleotides in the library of nucleic acids.

135. The method of claim 122, wherein one or more of steps (a)-(d) is performed in a reaction vessel, and the method further comprises adding carrier nucleic acids to the reaction vessel.


SEQUENCE LISTING (6 sequences)

SEQ ID NO: 1 - DNA, 15 bp, synthetic construct
  source 1..15: mol_type = other DNA
gtctcgtgggctcgg

SEQ ID NO: 2 - DNA, 60 bp, synthetic construct
  source 1..60: mol_type = other DNA
aaactctctctctaaactctctaaactctctaaactctctaaactctctctctaaactct

SEQ ID NO: 3 - DNA, 40 bp, synthetic construct
  source 1..40: mol_type = other DNA
aaactctctctctaaactctctaaactctctaaactctct

SEQ ID NO: 4 - DNA, 20 bp, synthetic construct
  source 1..20: mol_type = other DNA
aaactctctctctaaactct

SEQ ID NO: 5 - DNA, 20 bp, synthetic construct
  source 1..20: mol_type = other DNA
aatgatacggcgaccaccga

SEQ ID NO: 6 - DNA, 24 bp, synthetic construct
  source 1..24: mol_type = other DNA
caagcagaagacggcatacgagat